(19) United States
(12) Patent Application Publication
(10) Pub. No.: US 2021/0379085 A1
(43) Pub. Date: Dec. 9, 2021

(54) HALOGENATED SALICYLANILIDES FOR TREATING THE SYMPTOMS OF DERMATITIS

(71) Applicants: Union Therapeutics A/S, Hellerup (DK); Ceva Santé Animale S.A., Libourne, Cedex (FR)

(72) Inventors: Morten Otto Alexander Sommer, Hellerup (DK); Claudine Zemirline, Libourne, Cedex (FR); Pascal Butty, Libourne, Cedex (DK); Jérôme Guyonnet, Libourne, Cedex (DK)

(21) Appl. No.: 17/290,353

(22) PCT Filed: Nov. 1, 2019
(86) PCT No.: PCT/EP2019/080000

§ 371 Date: Apr. 30, 2021

(30) Foreign Application Priority Data
Nov. 2, 2018 (EP) 18306443.5

(51) Int. Cl. A61K 31/609; A61K 9/00; A61K 47/10

(52) U.S. Cl. CPC A61K 31/609; A61K 47/10; A61K 9/0014

(57) ABSTRACT

The present invention relates to halogenated salicylanilides for use in the treatment of dermatitis in a non-human subject, for example canine or feline atopic dermatitis.

### TREATMENT

This invention relates to a halogenated salicylanilide for use in the treatment of dermatitis in a non-human subject, for example the treatment of atopic dermatitis in a non-human subject.

### BACKGROUND

Dermatitis is an inflammatory skin condition characterized by one or more of erythema, pruritus, scaling, oozing, crusting and vesicles. There are numerous forms of dermatitis, with atopic dermatitis being the most common, particularly in dogs and cats.

Atopic dermatitis (AD) is an inflammatory condition of the skin characterized by erythema, pruritus, scaling, lichenification, and papulovesicles. AD is a complex condition associated with an impaired innate immune response in which the skin barrier at the site of lesions is compromised enabling triggers such as irritants, allergens, dust mites, bacteria and/or foods to penetrate the skin and initiate an inflammatory reaction. The initial inflammatory response in atopic dermatitis is thought to be mediated predominantly by Th2 (Bieber T. Atopic dermatitis. N. Engl. J. Med. 2008; 358(14):1483-94).

Symptoms of AD include patches of skin that are red or brownish, dry, cracked or scaly. A particularly problematic symptom of AD is pruritus (itchy skin), which can have a significant effect on a subject's quality of life including sleep deprivation, and psychiatric effects including depression and anxiety. In animals the pruritus can lead to the animal repeatedly scratching the dermatic lesions resulting in further damage to the already compromised epithelial barrier, self-inflicted alopecia and worsening of the dermatitis symptoms.

The compromised barrier function of the skin also results in dermatitis lesions being prone to bacterial infection, particularly by Staphylococcus aureus. The bacterial colonisation and infection of skin lesions has been linked with the inflammatory response in AD. Lesion colonization by S. aureus is a significant factor in the pathogenesis of atopic dermatitis for recurrent complications that exacerbate the disorder. Its presence, even without overt infection, appears to trigger multiple inflammatory reactions, via toxins, that act as super antigens and exogenous protease inhibitors that further damage the epidermal barrier and potentiate allergen penetration. (Bieber T. Atopic dermatitis. N. Engl. J. Med. 2008; 358(14):1483-94).

Current treatments for dermatitis such as AD typically target one or more symptoms of the dermatitis and include, the use of skin emollients (e.g. moisturisers and oils) to moisturise the skin, topical corticosteroids, anti-histamines to relieve itching and antibiotics including clindamycin, dicloxacillin, first-generation cephalosporins and macrolide antibiotics to treat secondary infections of skin lesions. Patients may also be treated with an immunosuppressant such as cyclosporin, tacrolimus or azathioprine. Phototherapy is also employed as a second-line treatment after failure of first-line treatments (Sidbury et al. Guidelines of care for the management of atopic dermatitis: section 3. J Am Acad. Dermatol. 2014 August; 71(2):327-49).

Topical corticosteroids can be effective in reducing inflammation and certain other symptoms of dermatitis, such as AD. However, the chronic use of topical corticosteroids are associated with undesirable side-effects, particularly skin atrophy.

Recently, dupilumab, was approved by the FDA for the treatment of adult patients with moderate-to-severe atopic dermatitis whose disease is not adequately controlled with topical prescription therapies. Dupiliumab, inhibits interleukin-4 and interleukin-13 signalling by binding to interleukin-4 receptor a.

The nonsteroidal phosphodiesterase 4 (PDE4) inhibitor crisaborole ointment was approved by the FDA in 2016 for the topical treatment of mild to moderate atopic dermatitis (AD) in human patients two years of age and older.

Apoquel® (oclacitinib maleate) is a Janus Kinase (JAK) inhibitor and is approved for use in the control of pruritus associated with allergic dermatitis for the control of atopic dermatitis in dogs.

Cytopoint® is a caninized monoclonal antibody to IL-31 and has been approved for use in the reduction of clinical signs associated with atopic dermatitis in dogs.

However, there remains a need for new treatments for dermatitis, particularly AD in non-human subjects.

The halogenated salicylanilides are a series of compounds including niclosamide, closantel, rafoxanide and oxyclozanide.

Niclosamide is approved for use as an anthelmintic drug for human and veterinary medicine. Niclosamide is a known taenicide effective against several parasitic tapeworms of livestock and pets (e.g. Taenia spp., Moniezia spp.) and also against rumen flukes (Paramphistomum spp.) and blood flukes (Schistosoma spp.). Niclosamide has also been shown to prevent the penetration of Schistosoma mansoni through the human skin. As well as used as an anticancer drug, pesticide and as an anti-trypanosoma drug. Niclosamide has also been shown to inhibit viral replication in human cells. (Ofori-Adjei et al; The International Journal of Risk & Safety in Medicine. 2008; 20:113-22; and Pearson et al; Annals of Internal Medicine. 1985; 102(4):550-1).

Oxyclozanide (CAS no. 2277-92-1) is used for the oral treatment and control of fascioliasis in cattle, sheep and goats (European Medicines Agency outcome or referral procedure report EMA/586006/2017, dated 28 Sep. 2017).

GB 2,456,376 and WO 2008/155535 describes the use of halogenated salicylanilides for the treatment of acne caused by propionibacteria.

WO 2016/038035 discloses the use of halogenated salicylanilides for the topical treatment of diseases or infections caused by Gram-positive bacteria.

Wu et al. (“Antihelminthic niclosamide modulates dendritic cells activation and function”, Cellular Immunology, 288(1-2): 15-23 (2014) discloses that niclosamide has an inhibitory action on lipopolysaccharide (LPS)-induced dendritic cell maturation and cytokine costimulatory molecule and MHC molecule expression in-vitro. It was also found that niclosamide-treated dendritic cells inhibited antigen specific T cell responses. The reference postulates that niclosamide may be useful for the treatment of chronic inflammatory disorders or dendritic cell mediated autoimmune disease, however, no clinical data is provided and the conclusions of the paper indicate that further studies are required to better understand the molecular mechanisms associated with the compound.

WO2019/053180, published after the priority date of this patent application, discloses a topical composition comprising oxyclozanide or niclosamide and dimethyl sulfoxide. The compositions are stated to be useful for the topical treatment of pyoderma or dermatitis in non-human mammals.

### BRIEF SUMMARY OF THE DISCLOSURE

In accordance with the present invention there is provided a halogenated salicylanilide, or a pharmaceutically acceptable salt or hydrate thereof, for use in the treatment of dermatitis (e.g. atopic dermatitis) in a non-human subject.

In embodiments there is provided a halogenated salicylanilide, or a pharmaceutically acceptable salt or hydrate thereof, for use in the treatment of dermatitis (e.g. atopic dermatitis) in a non-human subject to reduce or eliminate one or more of pruritus, erythema, induration, excoriation, lichenification, scaling, oozing, crusting, xerosis, exfoliation, lesion nodules, prurigo nodules, lesion vesicles, lesion papules, lesion plaques or lesion swelling associated with the dermatitis (e.g. atopic dermatitis).

In some embodiments the dermatitis (or eczema) is selected from: topic dermatitis, contact dermatitis, allergic contact dermatitis, irritant contact dermatitis, atopic dermatitis, seborrhoeic dermatitis, actinic dermatitis, pododermatitis, demodicosis, pompholyx dermatitis, lichen simplex chronicus (including Canine acral lick dermatitis and neurodermatitis), digital dermatitis (including bovine digital dermatitis), exfoliative dermatitis (drythroderma), carcinomatous dermatitis, nummular dermatitis, stasis dermatitis, flea allergy dermatitis, otitis, food allergic dermatitis, malassezia dermatitis, intertrigo, perioral dermatitis, dermatomyositis, eczematous dermatitis, photoallergic dermatitis, phototoxic dermatitis, phytophotodermatitis or radiation-induced dermatitis.

In some embodiments the dermatitis (or eczema) is selected from: topic dermatitis, contact dermatitis, allergic contact dermatitis, irritant contact dermatitis, atopic dermatitis, seborrhoeic dermatitis, pododermatitis, demodicosis, neurodermatitis, exfoliative dermatitis, carcinomatous dermatitis, flea allergy dermatitis, otitis, food allergic dermatitis, malassezia dermatitis, intertrigo, perioral dermatitis and dermatomyositis.

In some embodiments the dermatitis is atopic dermatitis.

The halogenated salicylanilide may reduce or eliminate one or more of pruritus, erythema, induration, excoriation, lichenification, scaling, oozing, crusting, xerosis, lesion nodules, prurigo nodules, lesion vesicles, lesion papules, lesion plaques and lesion swelling associated with the dermatitis (e.g. AD).

In some embodiments the halogenated salicylanilide may reduce or eliminate one or more of pruritus, erythema, induration, excoriation, lichenification, xerosis, lesion nodules, prurigo nodules, lesion vesicles, lesion papules or lesion swelling associated with the dermatitis (e.g. AD).

A particular problem associated with dermatitis, particularly AD, is pruritus (itching). This symptom of the disease is unpleasant for afflicted subjects and often results in one or more of stress, anxiety, disturbed sleep, sleep deprivation and psychiatric effects including depression and anxiety, leading to impaired quality of life. Animals are also prone to scratching lesions in an attempt to relieve the pruritus, however, this further damages the already compromised skin of the lesion leading to excoriation, increased erythema, induration and/or swelling. The additional damage to the barrier function of the skin associated with scratching the lesions also enhances exposure to allergens and irritants that can trigger an exacerbation of the dermatitis. Scratching of the lesions also increased the risk of infection of the dermatitis. Accordingly, in embodiments of the invention the halogenated salicylanilide is for use in reducing or eliminating pruritus associated with dermatitis (e.g. AD).

The pruritus in a subject may be assessed using a suitable scoring system for the pruritus associated with the dermatitis. It may be that the topical treatment of the dermatitis using the halogenated salicylanilide results in a reduction in the pruritus score compared to the score immediately prior to treatment of the subject.

The halogenated salicylanilide may be for use in the treatment of mild dermatitis (e.g. mild AD).

The halogenated salicylanilide may be for use in the treatment of moderate dermatitis (e.g. moderate AD).

The halogenated salicylanilide may be for use in the treatment of severe dermatitis (e.g. severe AD).

The halogenated salicylanilide may be for use in the treatment of moderate to severe dermatitis (e.g. moderate to severe AD).

The halogenated salicylanilide may be for use in the treatment of mild to moderate dermatitis (e.g. mild to moderate AD).

The severity of the dermatitis may be assessed using known methods. For example a suitable scoring system that assesses the clinical signs of the dermatitis on the subject. One such scoring method suitable for determining the severity of AD is the Canine Atopic Dermatitis Extent and Severity Index (CADESI), for example CADESI-01, CADESI-02 or CADESI-03 (Olivry et al. Validation of CADESI-03, a severity scale for clinical trials enrolling dogs with atopic dermatitis. Veterinary Dermatology, 18: 78-86), or CADESI-04 (Olivry T et al, Vet Dermatol. 2014 April; 25(2):77-85). CADESI-4 is currently recommended by ICADA (International Committee on Allergic Diseases of Animals) and is a preferred scoring system for AD. These scoring systems may also be used to grade other, similar forms of dermatitis.

The CADESI scores quantitatively describe the dog's skin condition, separately scoring areas of a dog's body for erythema, lichenification, and/or excoriation as ‘Normal or absent’ (0), ‘Mild’ (1), ‘Moderate’ (2), or ‘Severe’ (3). CADESI-03 differs from CADESI-02 in that it has an increased number of body sites assessed from 40 to 62, and includes another clinical sign (self-induced alopecia) and each sign is graded in a wider scale (scale of 0 to 5). CADESI-03. CADESI-04 requires only 20 defined body sites and takes approximately 33% of the time to conduct as compared to CADESI-03. Accordingly, a preferred dermatitis scoring system is CADESI-04.

It may be that the CADESI score (e.g. CADESI-03 or preferably CADESI-04 score) is reduced by 2, 4, 6 or 8 points compared to the score immediately before commencing treatment (the baseline score).

AD is characterised by an acute phase and a chronic phase. Acute AD is thought to be predominantly driven by Th2, whereas there is a switch to Th1 in the chronic stages of the disease (Gittler et al. J Allergy Clin Immunol. 2012 December; 130(6): 1344-1354) Acute AD lesions are typically bright red, “wet” and flat, becoming dull red, dry and thick with chronicity.

The halogenated salicylanilide may be for use in the treatment of acute AD. For example, the halogenated salicylanilide may be for use in the treatment or prevention of lesion redness (erythema, inflammation), induration, papulation, pruritus or excoriation in a non-human subject with acute AD. The acute AD may be mild, moderate or severe acute AD, for example moderate to severe acute AD or mild to moderate AD.

The halogenated salicylanilide may be for use in the treatment of a chronic form of dermatitis (e.g. chronic AD). For example, the halogenated salicylanilide may be for use in the treatment or prevention of Lichenification (for example, lined skin or prurigo nodules), pruritus or excoriation in a non-human subject with chronic AD. The chronic AD may be mild, moderate or severe chronic AD, for example moderate or severe chronic AD.

It may be that the dermatitis lesions are colonized by bacteria, for example the lesion may be colonized by Gram-positive bacteria. In certain embodiments the halogenated salicylanilide is for use in the treatment of a dermatitis lesion (e.g. an AD lesion) that is colonized by Gram-positive bacteria. The Gram-positive bacteria that may colonize the lesion include, but are not limited to Staphylococcus spp., Streptococcus spp. Propionibacterium spp. or Corynebacterium spp. In some embodiments the Gram-positive bacteria are selected from Staphylococcus spp. In some embodiments the Gram-positive bacteria are selected from selected from Staphylococcus aureus, Streptococcus pyogenes and Propionibacterium acnes. In some embodiments the Gram-positive bacteria are selected from Streptococcus uberis, Staphylococcus aureus, Staphylococcus pseudintermedius, Staphylococcus intermedius, Staphylococcus schleiferi, and coagulase-positive staphylococci. In some embodiments the Gram-positive bacteria are selected from Staphylococcus aureus, Staphylococcus pseudintermedius, Staphylococcus intermedius, Staphylococcus schleiferi and Staphylococcus hyicus. In a preferred embodiment bacteria are Staphylococcus pseudintermedius. For example, the bacteria may be a strain that is resistant to methicillin, e.g., methicillin resistant Staphylococcus aureus (MRSA), methicillin resistant Staphylococcus pseudintermedius (MRSP) or methicillin resistant Staphylococcus intermedius (MRSI).

In other embodiments the dermatitis lesion is not colonized by bacteria. Reference to “not colonized” means that the lesion is substantially free from bacteria, for example the lesion to be treated in the subject carries less than 1000 CFU/cm². The CFU in a sample taken from the lesion may be determined using conventional cell culturing methods. The sample could be, for example, a swab or skin biopsy obtained from the lesion. Accordingly, it may be that the halogenated salicylanilide is for use in the treatment of dermatitis (e.g. AD) that is not colonized or infected by bacteria, for example the AD lesion is not colonized or infected with Gram-positive bacteria.

Subjects with certain forms of dermatitis, including AD, are prone to exacerbation (flares) in their dermatitis. In the case of AD a flare could result from, for example, exposure to an irritant or allergen or a change in ambient conditions such as elevated temperature or humidity. Accordingly, the halogenated salicylanilide may be useful in the prevention or treatment of exacerbations of dermatitis (e.g. AD) in a non-human subject. It may be that the halogenated salicylanilide is for use in reducing the frequency of exacerbations of dermatitis (e.g. AD) in a non-human subject. It may be that the halogenated salicylanilide is for use in reducing the severity of an exacerbation of dermatitis (e.g. AD) in a non-human subject. It may be that the halogenated salicylanilide is for use in reducing the duration of an exacerbation of dermatitis (e.g. AD) in a non-human subject.

Accordingly, in embodiments the halogenated salicylanilide is for use in the treatment of an exacerbation of dermatitis (e.g. AD) in a non-human subject. In embodiments the halogenated salicylanilide is for use in preventing or reducing the frequency of dermatitis (e.g. AD) exacerbations in a non-human subject. In embodiments the halogenated salicylanilide is for use in reducing the severity of exacerbations of dermatitis (e.g. AD) in a non-human subject.

In the embodiments described herein that refer to exacerbations of the dermatitis, the exacerbation may be an exacerbation of one or more of the symptoms of the dermatitis described herein (e.g. an exacerbation of one or more of pruritus, erythema, induration or excoriation).

A further aspect of the invention provides a method of treating dermatitis (e.g. AD) in a non-human subject, the method comprising administering to the subject a therapeutically effective amount of a halogenated salicylanilide, or a pharmaceutically acceptable salt or hydrate thereof. The method is applicable to all aspects of the treatment of dermatitis (e.g. AD) described herein.

A further aspect of the invention provides the use of a halogenated salicylanilide, or a pharmaceutically acceptable salt or hydrate thereof in the manufacture of a medicament for the treatment of dermatitis (e.g. AD) in a non-human subject. The use is applicable to all aspects of the treatment of dermatitis (e.g. AD) described herein.

In some embodiments the subject is a companion animal, for example a dog or a cat.

Halogenated salicylanilides are also known as 2-hydroxy-N-phenylbenzamides or 2-hydroxybenzanilides. Salicylanilides are weakly acidic phenolic compounds. Halogenated salicylanilides are salicylanilides substituted by at least one halo group. A number of halogenated salicylanilide derivatives are known. Any halogenated salicylanilide possessing an effect on AD may be used in the present invention. For example, the halogenated salicylanilide may be any of the niclosamide analogues described in WO 2008/021088, which are incorporated herein by reference thereto.

The halogenated salicylanilide may be a halogenated salicylanilide of the formula (I):

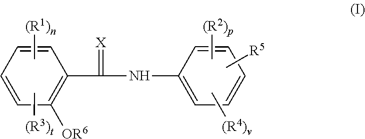

wherein
X is O or S;
R¹ and R² are at each occurrence independently selected from halo;
R³ and R⁴ are at each occurrence independently selected from H, C₁₋₆ alkyl, C₁₋₆ haloalkyl, —OR′, —NO₂ and —CN;
R⁵ is H or -L¹-R⁷;
R⁶ is H or —C(O)R^{A2};
L¹ is selected from a bond, O, S, or —(CR^{A3}R^{B})ₒ—, wherein o is 1 or 2;
R⁷ is phenyl, unsubstituted or substituted with 1, 2, or 3 groups selected from halo, C₁₋₄ alkyl, C₁₋₄ haloalkyl, —OR^{A4}, —NO₂ and —CN;
R_{A1}, R^{A2}, R^{A3} and R^{A4} are at each occurrence independently selected from H and C₁₋₄ alkyl;
R^{B} is at each occurrence selected from H, C₁₋₄ alkyl and —CN;
n and p are each independently selected from 0, 1, 2, 3 or 4, with the proviso that n+p is at least 1;
t and v are independently selected from 0, 1 and 2;
   or a pharmaceutically acceptable salt, or ester or hydrate thereof.

In some embodiments the halogenated salicylanilide is selected from niclosamide, closantel, oxyclozanide and rafoxanide, or a pharmaceutically acceptable salt or hydrate thereof. It may be that the halogenated salicylanilide is niclosamide or a pharmaceutically acceptable salt thereof. It may be that the halogenated salicylanilide is niclosamide or a hydrate thereof. It may be that the halogenated salicylanilide is niclosamide. In some embodiments the halogenated salicylanilide is anhydrous niclosamide.

The halogenated salicylanilide may be administered using any suitable route of administration, for example orally, topically, parenterally (for example intravenous, subcutaneous, intramuscular or intraperitoneal dosing) or as a suppository for rectal dosing).

In a particular embodiment the halogenated salicylanilide is topically administered to the subject. Suitably the halogenated salicylanilide is topically administered directly to an AD lesion on the subject. When the halogenated salicylanilide is topically administered it is suitably administered in the form of a pharmaceutical composition in a dosage form suitable for topical administration, for example as a cream, ointment, gel, foam, or aqueous, non-aqueous or oily solution or suspension. In particular embodiments the halogenated salicylanilide is formulated as a non-aqueous pharmaceutical composition suitable for topical administration, for example a non-aqueous cream, ointment, gel, lotion, or foam comprising the halogenated salicylanilide (for example niclosamide or a pharmaceutically acceptable salt or hydrate thereof). In some embodiments the halogenated salicylanilide is formulated as an aqueous pharmaceutical composition suitable for topical administration, for example an aqueous cream, ointment, gel, lotion, or foam comprising the halogenated salicylanilide (for example niclosamide or a pharmaceutically acceptable salt or hydrate thereof or oxyclozanide or a pharmaceutically acceptable salt or hydrate thereof).

In some embodiments the halogenated salicylanilide (e.g. niclosamide or a pharmaceutically acceptable salt or hydrate thereof or oxyclozanide or a pharmaceutically acceptable salt or hydrate thereof) is formulated as a spot-on or line-on formulation.

In certain embodiments the halogenated salicylanilide is formulated as a topical composition comprising the halogenated salicylanilide (for example, selected from niclosamide, rafoxanide, oxyclozanide and closantel, or a pharmaceutically acceptable salt thereof of hydrate thereof); and polyethylene glycol (PEG).

In certain embodiments the halogenated salicylanilide is formulated as a topical composition comprising the halogenated salicylanilide (for example, selected from niclosamide, rafoxanide, oxyclozanide and closantel, or a pharmaceutically acceptable salt thereof of hydrate thereof) and a non-polymeric glycol (for example an alkylene glycol, e.g. a C₂₋₈ alkylene glycol such as propylene glycol).

In certain embodiments the halogenated salicylanilide is formulated as a topical composition comprising the halogenated salicylanilide (for example, selected from niclosamide, rafoxanide, oxyclozanide and closantel, or a pharmaceutically acceptable salt thereof of hydrate thereof) and a glycol ether, for example 2-(2-ethoxyethoxy)ethanol (Transcutol).

In certain embodiments the halogenated salicylanilide is formulated as a non-aqueous topical composition comprising:
(i) a halogenated salicylanilide (for example, selected from niclosamide, rafoxanide, oxyclozanide and closantel, or a pharmaceutically acceptable salt thereof of hydrate thereof); and
(ii) polyethylene glycol (PEG).

In certain embodiments the halogenated salicylanilide is formulated as a non-aqueous topical gel composition comprising a halogenated salicylanilide (for example, selected from niclosamide, rafoxanide, oxyclozanide and closantel, or a pharmaceutically acceptable salt thereof of hydrate thereof) and a gel forming agent. The gel-forming agent may be any of the gel-forming agents disclosed herein. Suitably the topical gel composition further comprises a PEG.

Suitably the PEG in the composition is selected such that the composition together with any other components of the composition (e.g. in the form of a liquid, semi-solid or gel composition) can easily be applied to, spread over and/or rubbed into the skin. It may be that the PEG has a melting point that is less than 35° C. In certain embodiments the PEG is selected such that it is soft or, suitably molten at body temperature. For example, the PEG may have a melting point of 32° C. or less, or less than 30° C., or less than 25° C.

It may be that the halogenated salicylanilide is present in an amount of up to 15% by weight of the compositions described herein, for example from 0.05% to 10% by weight of the composition, from 0.05% to 4.5% by weight, from 1% to 3% by weight, from 1.5% to 4.5% by weight. For example, at about 2% by weight of the composition or at about 4% by weight of the composition.

It may be that the topical composition comprising the halogenated salicylanilide provides a local pH of greater than 4.5 at the site of application of the composition (for example an AD lesion). It may be that the composition provides a local pH of less than 6 at the site of application following topical application of the composition. Suitably the composition provides a local pH in the range of from about 4.5 to about 6 at the site of topical application of the composition.

Also provided is a spot-on or line-on composition comprising the halogenated salicylanilide or a pharmaceutically acceptable salt or solvate thereof. Examples of such compositions are set out in the detailed description herein.

Further aspects and features of the invention are set out in the detailed description below.

### BRIEF DESCRIPTION OF DRAWINGS

FIG. 1 shows the changes in biomarker expression that correlated with TSS/TAA and were found to have significantly changed compared to vehicle and baseline (S100A12, S100A9, PI3, CXCL1 and S100A7) as analysed in skin biopsies taken at Day 22 in the study of Example 3.

FIGS. 2-5 show the changes in biomarker expression (KRT16, MMP12, IL13, CCL17, CCL22, IL8, LOR, FLG, CD11c Dermis, S100A8, S100A12, S100A7, S100A9, IL22, PI3, CXCL1, IL17A, IL19, CAMP and DEFB4A/DEFB4B) that were found to correlate with TSS and were found to have significantly changed compared to baseline as analysed in skin biopsies taken at Day 22 in the study of Example 3.

FIG. 6 shows the correlation between individual scores (erythema, edema/papulation, oozing/crusting, excoriation, lichenification and dryness) and TSS as found in the study of Example 3.

FIG. 7 shows the changes in expression of biomarkers (IL13, S100A7, S100A8, KRT16, IL22, S100A9, S100A12, CCL17, MMP12, PI3, CCL22, DEFB4A/DEFB4B, IL19 and LOR) that correlated with edema/papulation and were found to have significantly changed compared to baseline as analysed in skin biopsies taken at Day 22 in the study of Example 3.

FIG. 8 shows the changes in expression of biomarkers (S100A7, S100A9, KRT16, IL13, S100A8, DEFB4A/DEFB4B, PI3, CCL17, S100A12, IL22 and MMP12) that correlated with erythema and were found to have significantly changed compared to baseline as analysed in skin biopsies taken at Day 22 in the study of Example 3.

FIG. 9 shows the changes in expression of biomarkers (IL22, S100A7, S100A8, S100A12, DEFB4A/DEFB4B, S100A9 and LOR) that correlated with lichenification and were found to have significantly changed compared to baseline as analysed by in skin biopsies taken at Day 22 in the study of Example 3.

FIG. 10 shows the changes in expression of biomarkers (IL13) that correlated with dryness and were found to have significantly changed compared to baseline as analysed in skin biopsies taken at Day 22 in the study of Example 3.

FIG. 11 shows the changes in expression of biomarkers (IL8) that correlated with excoriation and were found to have significantly changed compared to baseline as analysed in skin biopsies at Day 22 in the study of Example 3.

FIGS. 12-15 show the changes in biomarker expression (KRT16, MMP12, IL13, CCL17, CCL22, IL8, LOR, FLG, S100A8, S100A12, S100A7, S100A9, IL22, PI3, DEFB4A/DEFB4B, IL19) that were found to correlate with TAA and were found to have significantly changed compared to baseline as analysed in skin biopsies taken at Day 22 in the study of Example 3.

FIGS. 16-25 show changes in biomarker expression (IL6, IL8, IL170, IL1B, IL15, IL15RA, IL2, CCLS, IFNG, CXCL9, IL12A/IL12p35, CXCL10, IL13, MO, IL33, TSLP-R, IL31, IL5, CCL17, CCL18, CCL22, CCL26, IL17A, IL17F, IL23A/IL23p19, CAMP/LL37, IL19, IL12B/IL23p40, DEFB4A/DEFB4B, CXCL1, CXCL2, CCL20, PI3, IL22, S100A7, S100A8, S100A9, S100A12, FLG, PPL, LOR, KRT16, MMP12, IL9 and FOXP3) for vehicle (A) and niclosamide (B) compared to baseline as analysed in skin biopsies taken at Day 22 in the study of Example 3. FCH stands for fold change.

FIGS. 26-29 show changes in cell markers (CD3, langerin, CD11c and FceR1) for vehicle (A) and niclosamide (B) compared to baseline as analysed in skin biopsies taken at Day 22 in the study of Example 3.

FIG. 30 illustrates the arithmetic profile of oxyclozanide (μg/g) in stratum corneum of skin flank following oral or topical administration in dogs in the study of Example 4.

FIG. 31 illustrates the mean plasma concentration-time of oxyclozanide (μg/L) obtained following topical administration in dogs in the study of Example 5.

FIG. 32 illustrates the mean (μg/g) skin biopsies concentration-time of oxyclozanide obtained following topical administration in dogs in the study of Example 5.

FIG. 33 illustrates mean (μg/g) stratum corneum (strips) concentration-time of oxyclozanide obtained following topical administration in dogs on 6 zones in the study of Example 5.

### DETAILED DESCRIPTION

### Definitions

Unless otherwise stated, the following terms used in the specification and claims have the following meanings set out below.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. For example, certain methods herein treat dermatitis (e.g. AD) by decreasing a symptom of dermatitis (e.g. AD). Symptoms of dermatitis are known or may be readily determined by a person of ordinary skill in the art. The term “treating” and conjugations thereof, include prevention of a pathology, condition, or disease (e.g. preventing the development of one or more symptoms of dermatitis (e.g. AD).

Reference to a treatment to “reduce or eliminate” one or more symptoms of dermatitis is to be understood to be “treatment” of those symptom(s). Thus reference to reducing or elimination a symptom includes the treatment of the symptom. Thus also included within the invention is a halogenated salicylanilide, or a pharmaceutical salt thereof, for use in the treatment of one or more symptoms associated with a dermatitis (e.g. AD) disclosed herein, for example the treatment of one or more of pruritus, erythema, induration, excoriation, lichenification, scaling, oozing, crusting, xerosis, exfoliation, lesion nodules, prurigo nodules, lesion vesicles, lesion papules, lesion plaques or lesion swelling associated with the dermatitis (e.g. atopic dermatitis). Thus it may be the halogenated salicylanilide is for use in the treatment of pruritis associated with dermatitis (e.g. AD). By way of another example the halogenated salicylanilide may be for use in the treatment of erythema associated with dermatitis (e.g. AD).

The term “associated” or “associated with” in the context of a substance or substance activity or function associated with a disease (e.g. AD) means that the disease is caused by (in whole or in part), or a symptom of the disease is caused by (in whole or in part) the substance or substance activity or function.

When a compound or salt described in this specification is administered to treat a disorder, a “therapeutically effective amount” is an amount sufficient to reduce or completely alleviate symptoms or other detrimental effects of the disorder; cure the disorder; reverse, completely stop, or slow the progress of the disorder; or reduce the risk of the disorder getting worse.

Colony-forming unit (CFU) is an approximate estimate of the number of viable bacterial cells in a sample. Viable is defined as the ability of the cell to multiply via binary fission under the controlled conditions.

The term “pharmaceutically acceptable salt” refers to salts that retain the biological effectiveness and properties of the compounds described herein and, which are not biologically or otherwise undesirable. Reference to pharmaceutically acceptable salts is intended to encompass all salt forms that are suitable for administration to a non-human subject and as such encompasses veterinarially acceptable salts. Pharmaceutically acceptable salts are well known to skilled persons in the art. Particular salts include ethanolamine or piperazine salts. Accordingly, it may be that a reference to a salt of a halogenated salicylanilide herein may refer to a pharmaceutically acceptable salt of the halogenated salicylanilide.

The term “solvate” is used herein to refer to a complex of solute, such as a compound or salt of the compound, and a solvent. If the solvent is water, the solvate may be termed a hydrate, for example a monohydrate, dihydrate, trihydrate etc., depending on the number of water molecules present per molecule of substrate. Reference to “a halogenated salicylanilide, or a pharmaceutically acceptable salt or hydrate thereof” includes hydrates of the halogenated salicylanilide and hydrates of a salt of the halogenated salicylanilide.

The term “halo” or “halogen” refers to one of the halogens, group 17 of the periodic table. In particular the term refers to fluorine, chlorine, bromine and iodine. Preferably, the term refers to fluorine, chlorine or bromine and particularly fluorine.

The term Cm-n refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁₋₄ alkyl” similarly refers to such groups containing up to 4 carbon atoms. The alkyl groups may be unsubstituted or substituted by one or more substituents. Substituents for the alkyl group may be halogen, e.g. fluorine, chlorine, bromine and iodine, OH, C₁₋₄ alkoxy.

The term “C₁₋₆-haloalkyl” refers to a C₁₋₆ alkyl group that is substituted by at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoroethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a C₁₋₆ alkyl group substituted with at least one fluorine atom, for example C₁₋₆ alkyl.

Reference to an “ester” of the halogenated salicylanilide refers to an ester (RC(O)O— or ROC(O)—) formed with an available hydroxy or carboxy group on the halogenated salicylanilide. For example, an ester formed by the esterification of the 2-hydroxy group of the benzamide in a halogenated salicylanilide. The ester may be cleavable following topical application of the salicylanilide to provide the free hydroxy or carboxy group of the parent molecule thereby providing a prodrug of the halogenated salicylanilide. The ester may be for example a C₁₋₆-alkyl ester.

Reference to an “alkyl monohydroxy alcohol” refers to an alkyl alcohol which has one hydroxyl group, representative examples of alkyl monohydroxy alcohols include short chain alkyl monohydroxy alcohols, particularly C₁₋₆-monohydroxy alcohols or C₁₋₄-monohydroxy alcohols, for example methanol, ethanol, propanol or isopropanol.

Reference to an “alkanol amine” refers to an amine N-substituted by one, two or three alkyl alcohol moieties (for example one, two or three C₁₋₄-alkyl alcohol moieties). Representative examples of alkanol amine include ethanolamine, diethanolamine, triethanolamine, isopropanolamine and diisopropanolamine.

Reference to “PEG x00” herein means a polyethylene glycol with an average molecular weight of x00. For example, PEG 400 refers to a PEG with an average molecular weight of 400. Unless stated otherwise reference herein to the molecular weight of polymer, such as a PEG is a reference to number average molecular weight (Mn) of the polymer. The number average molecular weight can be measured using well known methods, for example by gel permeation chromatography or 1H NMR end-group analysis. Such methods include GPC analysis as described in Guadalupe et al (Handbook of Polymer Synthesis, Characterization, and Processing, First Edition, 2013) and end group analysis described in e.g. Page et al Anal. Chem., 1964, 36 (10), pp 1981-1985.

The methods disclosed herein are directed to the treatment of dermatitis in non-human subjects. Reference to a “subject” or “patient” herein mean a non-human subject unless expressly stated otherwise.

The halogenated salicylanilide may be administered to the subject in the form of a prodrug of the halogenated salicylanilide. As used herein, the term “prodrug” refers to covalently bonded moiety on the halogenated salicylanilide which modifies the biological and/or physical properties of the compound. The active halogenated salicylanilide is released following administration (for example topical administration) of the prodrug compound. Prodrugs may be formed by, for example, modification of a suitable functional group in the parent compound, for example a carboxylic or hydroxy group may be modified to form an ester which is cleaved following topical application of the prodrug. Various prodrug strategies are known and are described in, for example, the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen
d) H. Bundgaard, Chapter 5 “Design and Application of Pro-drugs”, by H. Bundgaard p. 113-191 (1991); and
e) H. Bundgaard, Advanced Drug Delivery Reviews, 8, 1-38 (1992).

Unless stated otherwise, reference herein to a “% by weight of a halogenated salicylanilide or a pharmaceutically acceptable salt thereof” is intended to refer to the amount of the free acid (i.e. non-salt form) of the halogenated salicylanilide. For example, reference to a composition comprising “5% by weight of niclosamide or a pharmaceutically acceptable salt thereof” refers to a composition comprising 5% by weight of the niclosamide as the free acid. Accordingly, where such a composition comprises a salt of niclosamide, the absolute amount of the niclosamide salt in the composition will be higher than 5% by weight in view of the salt counter ion that will be also be present in the composition. Similarly reference to “% by weight/volume (% w/v) of a halogenated salicylanilide or a pharmaceutically acceptable salt thereof” refer to the concentration of the free acid (i.e. non-salt form) of the halogenated salicylanilide per unit volume and is calculated as the % w/v=(weight of the salicylanilide in g/volume in mL of the composition)*100%.

The term “gel” is used herein refers to a semi-solid, apparently homogeneous substance that may be elastic and jelly-like (as in gelatin). The gel comprises a three-dimensional polymeric or inorganic matrix within which is dispersed a liquid phase. The matrix of the gel comprises a network of physically or chemical cross-linked polymers or copolymers that swell but do not dissolve in the presence of a solvent (for example the low molecular weight PEG). The cross-linking within the gel matrix may be physical cross linking (for example by hydrogen bonding or ionic cross-linking) or may be covalently cross-linked. In some embodiments the gel composition is a non-aqueous gel compositions wherein the halogenated salicylanilide is dissolved or dispersed in a suitable non-aqueous medium (e.g. PEG). The non-aqueous medium/halogenated salicylanilide solution or dispersion is then dispersed within the polymeric cross-linked network of the gel. Alternatively, the halogenated salicylanilide may be dissolved or dispersed within the polymeric cross-linked network of the gel. The gels are preferably clear in appearance; however, turbid gels are also contemplated. Generally, the gel-forming agent, for example gel-forming polymer is present in the gel in an amount of from about 0.5-15% by weight, typically 0.5-2% by weight. The U.S.P. defines gels as a semi-solid system consisting of dispersion made up of either small inorganic particles or large organic molecule enclosing and interpenetrated by liquid.

Reference to a “non-aqueous” composition (e.g. a non-aqueous topical composition), means that the composition is anhydrous and therefore substantially water free. For example, the compositions disclosed herein including the gel, cream and foam compositions contain less than 5%, less than 1% or suitably less than 0.01%, preferably less than 0.001% by weight water. Preferred non-aqueous compositions are those which are anhydrous and contain no detectable water.

Protic organic solvents are those that are capable of hydrogen bonding. The most common examples of protic organic solvents include but are not limited to alcohols and carboxylic acids.

Aprotic organic solvents are those that are not capable of hydrogen bonding. Common aprotic organic solvents include but are not limited to ethers, dimethylformamide (DMF), dimethylsulfoxide (DMSO) and acetonitrile.

Reference to “about” in the context of a numerical is intended to encompass the value+/−10%. For example, about 20% includes the range of from 18% to 22%.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Halogenated Salicylanilide

Any halogenated salicylanilide that has a beneficial effect on a symptom of dermatitis (e.g. AD) may be used in the treatments of dermatitis described herein (e.g. AD).

It may be that the halogenated salicylanilide is a halogenated salicylanilide of the formula (I):

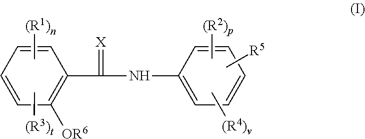

wherein
X is O or S;
R¹ and R² are at each occurrence independently selected from halo;
R³ and R⁴ are at each occurrence independently selected from H, C₁₋₆ alkyl, C₁₋₆ haloalkyl, —OR^{A1}, —NO₂ and —CN;
R⁵ is H or -L¹-R⁷;
R⁶ is H or —C(O)R^{A2};
L¹ is selected from a bond, O, S, or —(CR^{A3}R^{B})ₒ—, wherein o is 1 or 2;
R⁷ is phenyl, unsubstituted or substituted with 1, 2, or 3 groups selected from halo, C₁₋₄ alkyl, C₁₋₄ haloalkyl, —OR^{A4}, —NO₂ and —CN;
R^{A1}, R^{A2}, R^{A3} and R^{A4} are at each occurrence independently selected from H and C₁₋₄ alkyl;
R^{B} is at each occurrence selected from H, C₁₋₄ alkyl and —CN;
n and p are each independently selected from 0, 1, 2, 3 or 4, with the proviso that n+p is at least 1;
t and v are independently selected from 0, 1 and 2;
   or a pharmaceutically acceptable salt, or ester or hydrate thereof.

The following statements in the numbered paragraphs below apply to compounds of the formula (I). These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.
1. X is O.
2. R¹ and R² are at each occurrence independently selected from fluoro, chloro, bromo and iodo.
3. R¹ and R² are at each occurrence independently selected from chloro, bromo and iodo.
4. R¹ is chloro.
5. R¹ is bromo.
6. R¹ is iodo.
7. R² is chloro.
8. R² is bromo.
9. R² is iodo.
10. R³ and R⁴ are at each occurrence independently selected from H, C₁₋₄-alkyl, C₁₋₄-haloalkyl, —OR^{A1}, —NO₂ and —CN.
11. R³ and R⁴ are at each occurrence independently selected from H, C₁₋₄-alkyl, —OR^{A1} and —NO₂.
12. R³ and R⁴ are at each occurrence independently selected from H, C₁₋₄-alkyl, —CF₃, —OH, —OMe, —NO₂ and —CN, for example H, C₁₋₄-alkyl, —OH or —NO₂.
13. R⁴ is at each occurrence independently selected from —CF₃, —NO₂ and —CN.
14. R⁴ is at each occurrence independently selected from C₁₋₄-haloalkyl, —NO₂ and —CN.
15. R⁵ is H.
16. R⁵ is -L¹-R⁷.
17. L¹ is selected from —O—, —CH₂— and —CH(CN)—, for example —O— or —CH(CN)—.
18. R⁷ is phenyl, unsubstituted or substituted with 1, 2, or 3 groups selected from halo, C₁₋₄-alkyl, C₁₋₄-haloalkyl and —CN.
19. R⁷ is phenyl unsubstituted or substituted with 1, 2, or 3 groups (for example 1 or 2 groups) selected from halo.
20. R⁷ is unsubstituted phenyl.
21. L¹ is selected from —O— and —CH(CN)—; and R⁷ is phenyl unsubstituted or substituted with 1, 2, or 3 groups selected from halo.
22. R⁶ is H.
23. R⁶ is —C(O)R^{A2}, for example —O(O)CH₃.
24. t=0 or 1.
t=0.
26. v=0 or 1.
27. v=0.
28. o is 1.
29. v=1 and R⁴ is selected from —OH, C₁₋₄alkyl and —NO₂.
30. v=1 and R⁴ is selected from —CN, C₁₋₄haloalkyl (e.g. —CF₃) and —NO₂.
31. A compound of formula (I), or a pharmaceutically acceptable salt thereof.

Particular compounds are compounds of formula (I), or a pharmaceutically acceptable salt, hydrate or ester thereof wherein:
X is O;
R¹ and R² are at each occurrence independently selected from halo;
R³ and R⁴ are at each occurrence independently selected from H, C₁₋₄ alkyl, —OR′, —NO₂ and CN;
R⁵ is H or -L¹-R⁷;
R⁶ is H or —C(O)R^{A2};
L¹ is selected from O and —CH(CN)—;
R⁷ is phenyl unsubstituted or substituted with 1, 2, or 3 groups selected from halo;
R^{A1} and R^{A2} are at each occurrence independently selected from H and C₁₋₄-alkyl;
n and p are each independently selected from 0, 1, 2, 3 or 4, with the proviso that n+p is at least 1;
t and v are independently selected from 0, 1 and 2;
   or a pharmaceutically acceptable salt, or ester thereof.

It may be that the halogenated salicylanilide is selected from:

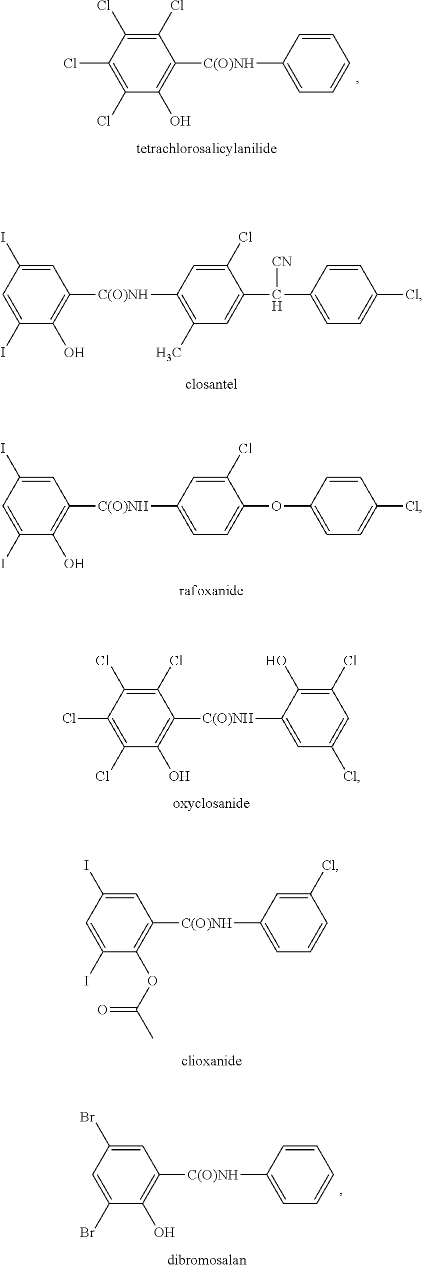

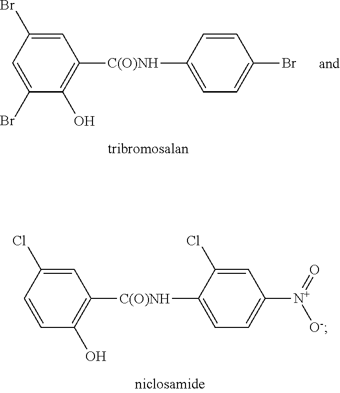

or a pharmaceutically acceptable salt or solvate (e.g. hydrate) thereof.

The halogenated salicylanilide may be a thioamide derivative, for example brotianide:

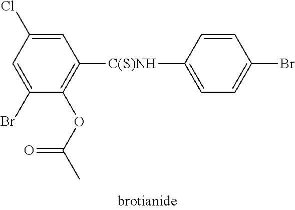

or a pharmaceutically acceptable salt, solvate (e.g. hydrate) thereof.

The halogenated salicylanilide may be selected from the group consisting of tetrachlorosalicylanilide, closantel, rafoxanide, oxyclozanide, resorantel, clioxanide, dibromosalan, tribromosalan, brotianide and niclosamide, or a pharmaceutically acceptable salt or prodrug or derivative thereof.

The halogenated salicylanilide may be selected from the group consisting of tetrachlorosalicylanilide, closantel, rafoxanide, oxyclozanide, resorantel, dibromosalan, tribromosalan and niclosamide, or a pharmaceutically acceptable salt or ester thereof.

The halogenated salicylanilide may be selected from the group consisting of clioxanide, closantel, oxyclozanide, rafoxanide, tribromosalan or a pharmaceutically acceptable salt or ester thereof.

The halogenated salicylanilide may be selected from the group consisting of tetrachlorosalicylanilide, closantel, rafoxanide, oxyclozanide, resorantel, clioxanide, dibromosalan, tribromosalan, brotianide and niclosamide, or a pharmaceutically acceptable salt or hydrate thereof.

The halogenated salicylanilide may be selected from the group consisting of tetrachlorosalicylanilide, closantel, rafoxanide, oxyclozanide, resorantel, clioxanide, dibromosalan, tribromosalan and niclosamide, or a pharmaceutically acceptable salt or hydrate thereof.

The halogenated salicylanilide may be selected from the group consisting of niclosamide, clioxanide, closantel, oxyclozanide, rafoxanide and tribromosalan, or a pharmaceutically acceptable salt or hydrate thereof.

The halogenated salicylanilide may be selected from the group consisting of clioxanide, closantel, oxyclozanide, rafoxanide and tribromosalan, or a pharmaceutically acceptable salt or hydrate thereof.

The halogenated salicylanilide may be selected from the group consisting of clioxanide, closantel, rafoxanide and tribromosalan, or a pharmaceutically acceptable salt or hydrate thereof.

The halogenated salicylanilide may be selected from the group consisting of niclosamide and oxyclozanide, or a pharmaceutically acceptable salt or hydrate thereof.

The halogenated salicylanilide may be selected from the group consisting of tetrachlorosalicylanilide, closantel, rafoxanide, oxyclozanide, resorantel, clioxanide, dibromosalan, tribromosalan, brotianide and niclosamide.

The halogenated salicylanilide may be selected from the group consisting of niclosamide, closantel, oxyclozanide and rafoxanide, or a pharmaceutically acceptable salt thereof.

The halogenated salicylanilide may be clioxanide, or a pharmaceutically acceptable salt or ester thereof, for example the halogenated salicylanilide is clioxanide or a pharmaceutically acceptable salt or hydrate thereof, suitably the halogenated salicylanilide is clioxanide.

The halogenated salicylanilide may be closantel, or a pharmaceutically acceptable salt or hydrate thereof, for example the halogenated salicylanilide is closantel or a pharmaceutically acceptable salt thereof, suitably the halogenated salicylanilide is closantel.

The halogenated salicylanilide may be oxyclozanide, or a pharmaceutically acceptable salt or ester thereof, for example the halogenated salicylanilide is oxyclozanide or a pharmaceutically acceptable salt or hydrate thereof, suitably the halogenated salicylanilide is oxyclozanide.

The halogenated salicylanilide may be rafoxanide, or a pharmaceutically acceptable salt or hydrate thereof, for example the halogenated salicylanilide is rafoxanide or a pharmaceutically acceptable salt thereof, suitably the halogenated salicylanilide is rafoxanide.

The halogenated salicylanilide may be tribromosalan, or a pharmaceutically acceptable salt or hydrate thereof, for example the halogenated salicylanilide is tribromosalan or a pharmaceutically acceptable salt thereof, suitably particularly the halogenated salicylanilide is tribromosalan.

The halogenated salicylanilide may be niclosamide, or a pharmaceutically acceptable salt or hydrate thereof, for example the halogenated salicylanilide is niclosamide or a pharmaceutically acceptable salt thereof.

In certain embodiments the halogenated salicylanilide is niclosamide in the free acid form.

In certain embodiments the halogenated salicylanilide is a pharmaceutically acceptable salt of niclosamide, for example an ethanolamine salt, or piperazine salt.

The halogenated salicylanilide may be a hydrate of niclosamide or pharmaceutically acceptable salt thereof. However, generally it is preferred that the niclosamide is not administered to the subject in the form of a hydrate. In certain embodiments the niclosamide is anhydrous niclosamide, or a pharmaceutically acceptable salt thereof. In a particular embodiment the niclosamide is anhydrous niclosamide.

### Pharmaceutical Compositions

The halogenated salicylanilide is suitably administered to the subject in the form of a pharmaceutical composition comprising the halogenated salicylanilide, or a pharmaceutically acceptable salt or hydrate thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, foams or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing). Preferably the halogenated salicylanilide is administered in the form of a topical pharmaceutical composition.

The halogenated salicylanilide is suitably compounded with an appropriate and convenient amount of excipients which may vary from about 5 to about 99 percent by weight of the total composition. The compositions may be prepared using conventional procedures well known in the art.

### Topical Pharmaceutical Compositions

In embodiments the halogenated salicylanilide is topically administered to the non-human subject for the treatment of dermatitis (e.g. AD).

In some embodiments the halogenated salicylanilide is present in an amount of up to 15% by weight of the compositions described herein, for example from 0.05% to 10% by weight of the composition, from 0.05% to 5% by weight, from 0.1% to 4.5% by weight, 0.1% to 7.5%, from 1% to 15% by weight, from 1% to 12% by weight, from 1% to 3% by weight, from 1.5% to 4.5% by weight, from 2% to 15% by weight, from 2% to 12% by weight, from 3% to 12% by weight, from 4% to 12% by weight, from 7% to 12% by weight or from 8 to 11% by weight of the composition It may be that the halogenated salicylanilide (e.g. oxyclozanide or niclosamide) is present in an amount of about 1%, about 2%, about 3%, about 4%, about 5%, about 6%, about 7%, about 8%, about 9%, about 10%, about 11%, about 12%, about 13%, about 14% or about 15% by weight of the composition. For example, the halogenated salicylanilide (e.g. oxyclozanide or niclosamide) is present in the composition at about 2% by weight of the composition or at about 4% by weight of the composition. It may be that the halogenated salicylanilide (e.g. oxyclozanide or niclosamide) is present in an amount of about 1%, about 2%, about 3%, about 4%, about 5%, about 6%, about 7%, about 8%, about 9%, about 10%, about 11%, about 12%, about 13%, about 14% or about 15% by weight/volume of the composition. In a particular embodiment the halogenated salicylanilide in oxyclozanide and is present in the composition in an amount of from 7 to 12% by weight/volume of the composition or about 9% to 11% by weight/volume of the composition, for example wherein the oxyclozanide is present in an amount of about 10% by weight/volume of the composition.

In certain embodiments the composition comprising the halogenated salicylanilide does not comprise dimethyl sulfoxide (DMSO).

In some embodiments the halogenated salicylanilide is present in the composition at a concentration of up to 250 mg/ml, for example 200 mg/ml or less, 150 mg/ml or less, 100 mg/ml or less or 50 mg/ml or less. For example, the halogenated salicylanilide (e.g. niclosamide or oxyclozanide) is present in the composition at a concentration of from 0.5 mg/ml to 200 mg/ml, 1 mg/ml to 150 mg/mL, 1 mg/mL to 120 mg/mL, 1 mg/mL to 100 mg/mL, 1 mg/mL to 50 mg/ml, 1 mg/mL to 20 mg/mL or 1 mg/mL to 10 mg/mL. In some embodiments the halogenated salicylanilide (e.g. niclosamide or oxyclozanide) is present in the composition at a concentration of from 50 mg/mL to 200 mg/mL, from 50 mg/mL to 200, or from 80 mg/mL to 120 mg/mL, for example at about 100 mg/mL.

In some embodiments the topical composition is an aqueous topical composition comprising the halogenated salicylanilide or pharmaceutically acceptable salt or hydrate thereof. The aqueous topical composition suitably comprises at least 5% by weight of water and one or more pharmaceutically acceptable excipients.

In other embodiments the topical composition is a non-aqueous topical composition comprising the halogenated salicylanilide or pharmaceutically acceptable salt or hydrate thereof.

The topical composition may be in any form suitable for topical administration, for example a cream, ointment, gel, foam, or aqueous, non-aqueous or oily solution or suspension comprising the halogenated salicylanilide. In some embodiments the topical composition may be in the form of an aqueous or non-aqueous gel comprising the halogenated salicylanilide and a gel forming agent. The gel forming agent may be any suitable gel-forming agent, including, but not limited to any of the gel forming agents described herein. In some embodiments the topical composition may be in the form of an aqueous cream or ointment comprising the halogenated salicylanilide and a suitable aqueous cream or non-aqueous ointment base. In some embodiments the topical composition may be in the form of a non-aqueous cream or ointment comprising the halogenated salicylanilide and a suitable non-aqueous cream or non-aqueous ointment base.

Suitably the topical composition is a spot-on, a pour-on or line-on topical composition. Spot-on compositions are applied to a single spot on the body of the animal suitably, between the animal's shoulders or neck. The active ingredients distribute through the epidermis to provide a therapeutically effective dose of the halogenated salicylanilide.

Reference to “Spot on” compositions herein refers to a composition comprising the halogenated salicylanilide wherein the composition is topically applied (preferably as a single unit dose) to a single localized area (i.e. a spot) on the skin of the subject.

Reference to “line on” compositions herein refers to a composition comprising the halogenated salicylanilide, wherein the composition is topically applied on the skin of the subject as a line or strip. Suitably line-on compositions are topically applied to the skin starting from the base of the tail along the spine to the shoulder blades, or from the middle of the back along the spine to the shoulder blades, or less of the subject (e.g. dog or cat). The length of the “line-on” application will depend on the subject being treated. For example, a line or strip about 30 cm, or 20 cm, or 15 cm, or 10 cm, or 5 cm long. Preferably the length of the line or strip is about 10 cm. Line on compositions may also be applied specifically around a specific skin area to be treated (e.g. an area of infected skin or a dermatitis lesion). Spot on or line on composition are suitably formulated as a unit dose adapted to the weight and/or size of the animal, wherein the entire dose is applied to the animal in a single application.

Pour-on or line-on compositions are suitably applied to the non-human subject as a line or strip to the skin on the subject. Such compositions are particularly suitable for large animals such as horses or cattle as well as small non-human mammals (e.g. dogs or cats). Pour on and lino on compositions are suitably applied against the grain of fur or hair of the non-human subject.

The topical composition may be prepared using known carriers or “bases” in which the halogenated salicylanilide is dissolved or dispersed. For example, the topical composition may comprise the halogenated salicylanilide dissolved or dispersed in a suitable base formulation selected from an oleaginous base (e.g. petrolatum, white petrolatum, yellow ointment or white ointment), an absorption base (e.g. hydrophilic petrolatum or lanolin), a water-removable base (oil in water emulsion); a water-soluble base (e.g. a polyethylene glycol).

### Non-Aqueous Topical Compositions

In particular embodiments the halogenated salicylanilide is formulated as a non-aqueous pharmaceutical composition suitable for topical administration. For example, a non-aqueous cream, ointment, gel or foam comprising the halogenated salicylanilide (for example niclosamide or a pharmaceutically acceptable salt or hydrate thereof).

In certain embodiments the non-aqueous topical composition comprises:
(i) a halogenated salicylanilide (for example selected from niclosamide, rafoxanide, oxyclozanide and closantel), or a pharmaceutically acceptable salt or hydrate thereof); and
(ii) polyethylene glycol (PEG), preferably a PEG with a melting point of less than 40° C.

In certain embodiments the non-aqueous composition comprises:
(i) a halogenated salicylanilide (for example selected from niclosamide, rafoxanide, oxyclozanide and closantel), or a pharmaceutically acceptable salt or hydrate thereof; and
(ii) greater than 60% by weight of a PEG, preferably wherein the average molecular weight of the PEG is 800 or less and particularly 600 or less. For example, the average molecular weight of the PEG is less than 800. It may be that the average molecular weight of the PEG is less than 400.

In certain embodiments, the composition further comprises a non-polymeric glycol (for example an alkylene glycol, e.g. a 02-8 alkylene glycol, preferably a 02-6 alkylene glycol and especially propylene glycol).

In certain embodiments the non-aqueous topical composition comprises propylene glycol. Accordingly the composition may comprise:
(i) a halogenated salicylanilide (for example selected from niclosamide, rafoxanide, oxyclozanide and closantel), or a pharmaceutically acceptable salt or hydrate thereof;
(ii) polyethylene glycol (PEG), (preferably a PEG with a melting point of less than 40° C.); and
(iii) a 02-8 alkylene glycol (preferably propylene glycol).

In certain embodiments the non-aqueous topical composition comprises:
(i) 0.1 to 5% by weight of a halogenated salicylanilide (e.g. selected from niclosamide, rafoxanide, oxyclozanide and closantel), or a pharmaceutically acceptable salt or hydrate thereof;
(ii) polyethylene glycol (PEG) with a melting point of less than 40° C.; and
(iii) 0.5 to 30% (for example 5 to 25%) by weight of a non-polymeric glycol (preferably propylene glycol).

Examples of PEG, preferably with an average molecular weight of less than 600, which may be used in the non-aqueous composition are described in more detail below under the section “Polyethylene Glycol (PEG)”

It may be that the non-aqueous composition comprises up to 10%, up to 20%, up to 30%, up to 35%, up to 40%, up to 45%, up to 50% or up to 55% by weight of PEG. For example, wherein the lower limit of PEG is 1% by weight and the upper limit is any of the values set out in this paragraph. For example, wherein the lower limit of PEG is 5% by weight and the upper limit is any of the values set out in this paragraph (e.g. a range of 5% to 20, 30, 40, 50, 60, 70, 80, 90 or 95% by weight PEG).

In some embodiments it has been found that a high concentration of PEG in the composition provides a non-aqueous topical composition with advantageous properties, for example one or more of improved dermal penetration and/or good tolerability when topically applied to the skin. Certain compositions described herein provide high concentration of the halogenated salicylanilide in skin tissues (e.g. the dermis and epidermis) and very low levels of systemic exposure (e.g. in the plasma) to the halogenated salicylanilide. The compositions are therefore expected to provide an effective local topical treatment of, for example, a dermal condition, with little or no systemic side-effects, because the systemic exposure is low. Such compositions are expected to provide a wide therapeutic window between the beneficial therapeutic effects and the onset of undesirable systemic side effects that may be associated with the halogenated salicylanilide. Such side effects could be systemic toxicity.

It may be that the non-aqueous composition comprises more than 65%, more than 70%, more than 75%, more than 80%, more than 85%, more than 90%, more than 95%, more than 96%, more than 97%, more than 98% or more than 99% PEG (preferably with an average molecular weight of 600 or less, for example a PEG with an average molecular weight of 400 or less); and wherein the % is by weight of the composition. Further amount of the PEG which may be present in the composition are described under the section “Polyethylene Glycol (PEG)”

It may be that the halogenated salicylanilide, or a pharmaceutically acceptable salt thereof is present in the non-aqueous composition in an amount of 0.01% to 10%, for example from 0.01% to 7.5%, from 0.01% to 7%, from 0.01% to 6.5%, from 0.01% to 6%, from 0.01% to 5.5%, 0.01% to 5%, from 0.01% to 4.5%, from 0.01% to 4%, from 0.01% to 3.5%, from 0.01% to 3%, from 0.1% to 5%, from 0.1% to 4.5%, from 0.1% to 4%, from 0.1% to 3.5%, from 0.1 to 3%, from 0.1 to 2.5%, from 0.1 to 2%, from 0.1 to 1.5%, from 0.1 to 1%, or from 0.5 to 3%, from 2% to 12%, from 4% to 12% or from 9% to about 11%, for example about 1%, about 2% about 2.5% about 3%, about 4%, about 4.5% about 5%, about 6% about 7% or about 10% wherein the % are by weight based upon the weight of the composition. Suitable examples of halogenated salicylanilides which may be used are described herein, for example, niclosamide, rafoxanide, oxyclozanide and closantel, or a pharmaceutically acceptable salt or hydrate thereof). The halogenated salicylanilides may be in the form of a hydrate, however, this is less preferred in the non-aqueous compositions described herein. Accordingly, it is preferred that the halogenated salicylanilide is in a substantially anhydrous form.

It may be that the non-aqueous composition of the invention comprises:
(i) 0.01 to 7.5%, such as 0.01 to 4.5% (e.g. 0.1 to 4%, or 0.1 to 3.5, or 0.1 to 3% or about 2%, or about 4%) by weight of a halogenated salicylanilide, or a pharmaceutically acceptable salt thereof; and
(ii) at least 70% (for example at least 90%) by weight of a PEG, wherein the average molecular weight of the PEG is 600 or less (for example less than 600 or from about 200 to about 600 or about 400).

It may be that the non-aqueous compositions described herein further comprise a polar organic solvent for example a polar organic solvent selected from an alkylene glycol (e.g. propylene glycol), 2-(2-ethoxyethoxy)ethanol, glycerol, a macrogol stearyl ether (e.g. macrogol 15 stearyl ether) or a macrogol isostearate or a fatty alcohol, for example a C₁₂-C₁₈-alcohol such as cetostearyl alcohol or a mixture two or more thereof. It may be that the polar organic is present in the composition in an amount of from about 5% to about 65%, about 10% to about 55% or about 25% to about 50% by weight of the composition.

It may be that the non-aqueous compositions described herein further comprise a glycol, for example an alkylene glycol (e.g. propylene glycol). It may be that the composition comprises from about 5% to about 30%, about 10% to about 30%, or about 14% to about 28% by weight of a glycol, particularly propylene glycol.

It may be that the non-aqueous compositions described herein further comprise 2-(2-ethoxyethoxy)ethanol. It may be that the composition comprises from about 1% to about 25%, about 5% to about 20% or about 10% to about 20% by weight of 2-(2-ethoxyethoxy)ethanol.

It may be that the non-aqueous compositions described herein further comprise glycerol. It may be that the composition comprises from about 5% to about 30%, about 10% to about 30%, or about 15% to 25% by weight of glycerol.

It may be that the composition comprises one or more non-polar excipients, for example one or more non-polar oils, hydrocarbon solvents or waxes. It may be that the composition comprises one or more non-polar excipients selected from aromatic or aliphatic esters, a mineral oil, a vegetable oil and long-chain or medium chain triglycerides. For example, the non-polar excipients may be selected from one or more of a mineral oil, (e.g. liquid paraffin or a paraffin wax) and medium chain triglycerides. It may be that the non-polar excipients are present in the composition in an amount of from about 2% to about 50%, about 5% to about 40%, about 5% to about 30%, or about 5% to 25% by weight of the composition.

It may be that the non-aqueous compositions described herein further comprise one or more surfactant or emulsifiers, for example an ionic or non-ionic surfactant or emulsifiers. Representative examples of surfactants or emulsifiers include any of those described herein, for example a PEGylated fatty acid glyceride (labrasol), polyoxyethylene glycol sorbitan alkyl ester (polysorbate), a polyoxyethylene glycol alkyl ether (Brij), polyoxyethylene ethers of fatty alcohols (ceteareth), or a fatty acid ester of glycerol (e.g. glyceryl stearate). It may be that the surfactant or emulsifiers are present in the composition in an amount of from about 0.1% to about 15%, about 0.2% to about 10%, or about 0.2% to about 5% by weight of the composition.

In certain embodiments the non-aqueous composition comprises a non-aqueous emulsion or microemulsion. Non-aqueous emulsion or microemulsion compositions are particularly suitable for providing compositions in the form of a non-aqueous topical cream composition. The non-aqueous emulsion comprise a non-aqueous hydrophilic phase (suitably comprising polar excipients) and a non-aqueous hydrophobic phase which is immiscible with the hydrophilic phase (suitably comprising non-polar excipients such as an oil). It may be that the hydrophilic phase comprises the continuous phase of the emulsion and the hydrophobic phase is dispersed within the hydrophilic phase as the discontinuous phase of the emulsion. In certain embodiments the non-aqueous hydrophobic phase comprises the continuous phase of the emulsion and the non-aqueous phase is dispersed within the non-aqueous hydrophobic phase as the discontinuous phase of the emulsion.

In certain embodiments the non-aqueous hydrophilic phase comprises the halogenated salicylanilide, the PEG and optionally one or more of the polar solvents described herein. Accordingly it may be that the non-aqueous hydrophilic phase comprises niclosamide, PEG and optionally one or more polar solvents selected from propylene glycol, 2-(2-ethoxyethoxy)ethanol, glycerol, a macrogol stearyl ether (e.g. macrogol 15 stearyl ether) and a fatty alcohol, for example a C₁₂-C₁₈-alcohol such as cetostearyl alcohol.

It may be that the non-aqueous hydrophobic phase of the emulsion or microemulsion comprises one or more of the non-polar excipients described herein, for example, a mineral oil, a vegetable oil and long-chain or medium chain triglycerides.

In those embodiments where the composition is in the form of a non-aqueous emulsion or microemulsion the composition suitably comprises a surfactant or emulsifier, for example one or more of the surfactants or emulsifiers described herein.

Suitably the non-aqueous composition comprises a solution of the halogenated salicylanilide. Accordingly, it is preferred that the halogenated salicylanilide is completely dissolved in the non-aqueous composition. However, it is contemplated that the halogenated salicylanilide may present as a dispersion in the composition. Alternatively, in some embodiments at least a proportion of the halogenated salicylanilide is dissolved in the composition. In this embodiment it is preferred that at least 80%, preferably at least 90%, more preferably at least 95% by weight of the halogenated salicylanilide is dissolved in the composition.

### Non-Aqueous Gel Compositions

In certain embodiments the non-aqueous topical composition of the invention is in the form of a non-aqueous topical gel composition

In certain embodiments there is provided a non-aqueous topical gel composition comprising:
(i) a halogenated salicylanilide (for example selected from niclosamide, rafoxanide, oxyclozanide and closantel), or a pharmaceutically acceptable salt or hydrate thereof; and
(ii) PEG with a melting point of less than 40° C.; and
(iii) a gel forming agent.

In certain embodiments there is provided a non-aqueous topical gel composition comprising:
(i) a halogenated salicylanilide (for example selected from niclosamide, rafoxanide, oxyclozanide and closantel), or a pharmaceutically acceptable salt or hydrate thereof;
(ii) greater than 60% by weight of a PEG, preferably wherein the average molecular weight of the PEG is less than 600; and
(iii) a gel-forming agent.

Particular aspects of the non-aqueous gel compositions are described below.

### Gel-Forming Agent

It may be that the gel-forming agent present in the compositions disclosed herein is an inorganic gel-forming agent. It may be that the gel-forming agent is a gel-forming polymer.

### Inorganic Gel Forming Agents

It may be that the gel-forming agent is an inorganic gel-forming agent, for example a bentonite or a silica. It may be that the gel-forming agent is magnesium aluminium silicate (Veegum®).

### Gel-Forming Polymers

The gel-forming agent may be a gel-forming polymer. The gel-forming polymer may be a hydrophilic gel-forming polymer. The gel-forming polymer may be selected from the group consisting of: gelatin; agar; agarose; pectin; carrageenan; chitosan; alginate; starch; starch components (e.g. amylose or amylopectin); tragacanth gum; xanthan gum; gum Arabic (acacia gum); guar gum; gellan gum; locust bean gum; polyurethane; polyether polyurethane; cellulose; cellulose ethers (for example methylcellulose, carboxymethyl cellulose, ethylcellulose, hydroxyethyl cellulose or hydroxypropyl cellulose), cellulose esters, cellulose acetates, cellulose triacetates; cross-bonded polyvinyl alcohol; polymers and copolymers of acrylic acid, hydroxyalkyl acrylates, hydroxyethyl acrylate, diethylene glycol monoacrylate, 2-hydroxypropylacrylate or 3-hydroxypropyl acrylate; carbomers (cross-linked poly(acrylic acids), for example carbomer 910, 934P, 940GE, 941GE, 971P, 974P; polymers and copolymers of methacrylic acid, hydroxyethyl methacrylate, diethyleneglycol monomethacrylate, 2-hydroxypropyl methacrylate, 3-hydroxypropyl methacrylate or dipropylene glycol monomethylacrylate; vinylpyrrolidone polymers; polymers and copolymers or acrylamide, N-methylacrylamide, N-propylacrylamide; methacrylamide, N-isopropylmethacrylamide, or N-2-hydroxyethylmethacrylamide; poloxamers (triblock copolymers comprising a central polyoxypropylene block flanked by two polyoxyethylene blocks, for example a Pluronic®); and gels comprising cross-linked polyalkylene glycols, for example gels comprising cross-linked polyethylene glycol or cross-linked polypropylene glycol. In specific embodiments binary or tertiary etc combinations of any of the above gel-forming agents are foreseen. When the gel forming agent comprises a PEG, the PEG is suitably a higher molecular weight than the PEG used as a solvent to dissolve or disperse the halogenated salicylanilide in the gel composition. Accordingly it is to be understood that when the gel-forming agent is a PEG, the PEG of the gel-forming agent is different to the PEG present in component (ii) of the compositions of the invention. For example, where the gel forming agent comprises a PEG, the PEG suitably has a molecular weight greater than 600, for example greater than 1000, greater than 10000 or greater than 20000. Suitably, when the gel forming agent comprises a PEG it has an average molecular weight of from about 600 to about 35,000, for example from about 800 to about 25,000, or from about 1000 to about 20,000. Other gel-forming agents are also contemplated, for example as disclosed in Gels handbook Vols 1-4, Osada et al. 2001 Elsevier.

The gel-forming polymer may be a gum, for example a gum selected from tragacanth gum, xanthan gum; gum arabic (acacia gum); guar gum; gellan gum locust bean gum.

The gel-forming polymer may be a cellulose ether, for example methylcellulose, carboxymethyl cellulose, ethylcellulose, hydroxyethyl cellulose, hydroxy propyl methyl cellulose or hydroxypropyl cellulose.

### Carbomer Gel-Forming Polymers

In a particular embodiment the gel-forming agent is a carbomer. Carbomers are high molecular weight cross-linked poly(acrylic acid) polymers. The polymers may be cross-linked by polyalcohol allyl ethers, for example, allyl sucrose or allyl pentaerythritol The carbomer may be a homopolymer, for example 910, 934P, 940GE, 941GE, 971P, 974P, wherein “GE” refers to medical grade and “P” oral grade. Derivatives of Carbomer polymers may also be used, for example Carbopol interpolymers comprising a carbomer polymer comprising a block copolymer of polyethylene glycol and a long chain alkyl acid ester, such derivatives are commercially available as ETD 2020 NF and Ultrez 10 NF from Lubrizol.

Carbomers (also known as Carbopols) are well known and are characterised in the United States Pharmacopeia/National Formulary (USP/NF) monograph for Carbomers and the European Pharmacopeia (Ph. Eur.) monograph for Carbomers, reference to which is incorporated herein.

The carbomer may have a viscosity of from about 4,000 to about 70,000, for example about 10,000 to about 60,000, for about 20,000 to about 50,000, about 25,000 to about 45,000 or about 29,400 to about 39,400 cP, wherein the viscosity is that of a 0.5 wt % solution of the carbomer in water, neutralised to pH 7.3-7.8 at 25° C., measured using a Brookfield RVT, 20 rpm, spindle #6.

Suitably the carbomer comprises from about 56% to about 68.0% by weight carboxylic acid (—COOH) groups. The proportion of carboxy groups present in the carbomer may be determined using known methods, for example by titrating an aqueous solution or dispersion of the polymer against NaOH.

Suitably the carbomer is substantially free of residual benzene (for example containing less than 0.5 parts per million). Accordingly, it is preferred that the carbomer is prepared without using benzene as a solvent during the polymerisation process. Preferred carbomers are those are prepared using ethyl acetate and optionally cyclohexane as the solvent during polymerisation.

A particular carbomer for use as a gelling agent in the present invention is Carbomer 974P. This carbomer suitably has a viscosity of 29400 to 39400 cP (0.5% solution in water neutralized to pH 7.3-7.8 and measured at 25° C. using a Brookfield RVT, 20 rpm with spindle #6). The carbomer typically has a carboxylic acid content of from 56 to 68%.

Conventionally carbomer gels are formed by dispersing the carbomer in water, which results in ionisation of the carboxy groups present in the polymer. The resulting solution or dispersion is then neutralised using a base, resulting in an increase in viscosity and gel formation. However, in the present invention the gel is a non-aqueous gel and gel formation may be achieved by dissolving or dispersing the carbopol in the organic solvent together with the halogenated salicylanilides and heating the mixture to about 70° C.

The gel-forming polymer may also be referred to as a colloid i.e. a colloid system wherein the colloid particles are disperse in the organic solvent and the quantity of solvent available allows for the formation of a gel. In embodiments it is preferred to use reversible colloids preferably thermo-reversible colloids (e.g. agar, agarose and gelatin etc.) as opposed to irreversible (single-state) colloids. Thermo-reversible colloids can exist in a gel and sol state, and alternate between states with the addition or elimination of heat. Thermoreversible colloids which may be used according to the invention, whether individually or in combination, include for example, gelatin, carrageenan, gelatin, agar, agarose (a polysaccharide obtained from agar), pectin and cellulose derivatives for example methylcellulose, carboxymethyl cellulose, ethylcellulose, hydroxyethyl cellulose, hydroxy propyl methyl cellulose or hydroxypropyl cellulose. Another term which may be applied to gel forming polymers is “thermotropic”: a thermotropic gelling agent is one caused to gel by a change in temperature. In embodiments of the invention, therefore, the gel former is a thermotropic gel-forming polymer or a combination of such polymers.

The gel-forming polymer may be or comprise an ionotropic gel-forming polymer whose gelling is induced by ions. Suitable ionotrophic gel-forming agents are anionic or cationic polymers which can be cross-linked by multivalent counter ions to form a gel. The ionotropic gel-forming polymers may be, for example chitosan, an alginate, carrageenan or pectin.

The gel-forming polymer may comprise or be a single gel-forming polymer or a mixture of two or more gel-forming polymers. For example, the gel-forming polymer may comprise a combination of two or more of the gel-forming polymers listed herein.

The amount of gel forming agent present in the composition should be selected so as to provide a gel composition having the required rheological properties, for example a viscosity suitable for topical application. Generally, the gel composition will be of a viscosity such that it can be readily dispensed and spread over and rubbed in the area of, for example, skin that is infected. The rheology of the gel composition will depend upon the particular gelling agent used, the molecular weight of the PEG, the particular halogenated salicylanilide and the amounts thereof in the composition. Generally, the gelling agent, for example a carbomer, will be present in the gel composition is an amount of up to about 10% by weight, for example up to about 1%, 2%, 3%, 4%, 5%, 5.5%, 6%, 6.5%, 7%, 7.5%, 8%, 8.5%. 9% or 9.5% by weight of the gel composition. Suitably the gelling agent, for example a carbomer, may be present in an amount of from about 0.01% to about 10% by weight of the gel composition, for example about 0.01% to about 8%, about 0.05% to about 7%, about 0.05% to about 6%, about 0.05% to about 5%, about 0.05% to about 4%, about 1% to about 6%, about 1% to about 5% or about 1% to about 4%, about 2% to about 5%, about 2% to about 4% or about 2% to about 3%, wherein the % is by weight based on the weight of the gel composition.

### Polyethylene Glycol (PEG)

In embodiments where PEG is present in the compositions comprising the halogenated salicylanilide described herein, the PEG suitably has one or more of the characteristics described in this section.

Suitably the PEG is liquid at ambient temperature (for example 20 to 25° C.), accordingly the solvent may be a low molecular weight PEG. Particularly, the PEG has an average molecular weight of 600 or less, suitably less than about 600. For example, the PEG may have an average molecular weight of from about 200 to about 600, about 200 to about 500 or about 200 to about 400. A particular PEG is selected from PEG 200, PEG 300 and PEG 400. In one particular embodiment the PEG is PEG 400. Alternatively, the PEG may comprise a mixture of PEGs which together with the other components of the composition provide a composition which is suitable for e.g. topical application to the subject. Accordingly, the PEG may be a mixture of one or more low molecular weight PEGs with one or more higher molecular weight PEG, wherein the mixture of PEGs has a melting point below 40, or preferably below about 37° C.

Suitably the PEG is present in an amount at least sufficient to provide a solution of the halogenated salicylanilide in the composition. As will be realised the amount of PEG required to dissolve the halogenated salicylanilide will depend upon the particular halogenated salicylanilide used and the other components of the composition. In certain embodiments the PEG is present in the composition of the invention an amount of at least 60%, suitably greater than 60% by weight of the composition. Non-aqueous compositions containing high amounts of PEG provide topical compositions which give high levels of the halogenated salicylanilide in skin tissues and only minimal systemic exposure to the halogenated salicylanilide. Such compositions have also been found to be well tolerated, despite containing high PEG concentrations. Suitably the PEG is present in an amount of greater than 60%, 65%, 70%, 75%, 80%, 85%, 90%, 91%, 92%, 93%, 94%, 95%, 96%, 97% 98% or 99% wherein the % is by weight based upon the weight of the composition. It may be that the PEG, preferably a PEG with an average molecular weight of 600 or less (particularly less than 600) is present in the non-aqueous composition of the invention in an amount of for example 65 to 98%, for example from 65% to 95%, 65% to 90%, 65% to 80%, 70% to 98%, 70% to 95%, 70% to 85%, 70% to 80%, 80% to 98%, 80% to 95%, 80% to 90%, 85% to 98% or 85% to 95%, wherein the % is by weight based upon the weight of the non-aqueous composition of the invention.

In certain embodiments the composition (e.g. a non-aqueous composition) comprise lower concentrations of PEG, for example 50% or less, 45% or less, 40% or less, 35% or less 30% or less, 25% or less, 20% or less, 15% or less, wherein the % is % by weight of the composition. It may be that the PEG is present from about 1% to about 50%, from about 5% to about 40%, from about 5% to about 35%, or from about 5 to about 30% by weight of the composition.

### Topical Foam Compositions

In certain embodiments the halogenated salicylanilide is formulated as a foam composition. The foam composition may be an aqueous foam composition such as an emulsion or nano-emulsion foams or a water-alcohol based foam (e.g. a water-ethanolic foam). Alternatively, the foam may be a non-aqueous (i.e. water-free) foam composition, including but not limited to oil-based foams, petrolatum-based foams, ointment foams;

emollient foams and foams formed using non-aqueous hydrophilic excipients. When the foam is a foam formed from an emulsion, the emulsion may be a water-in-oil emulsion or an oil-in-water emulsion comprising the halogenated salicylanilide. Foams suitable for the delivery of pharmaceuticals are well-known and are described in for example Arzhavitina et al, “Foams for pharmaceutical and cosmetic application” Int. J. Pharm., 394, 1-17 (2010).

Suitably the foam is a breakable foam, i.e. a thermally stable foam which collapses (breaks) upon application of shear stress to the foam. Such breakable foams can be applied to the skin as a foam and then collapse when the foam is rubbed into the skin, thereby enabling the active to be applied to the skin in the area required.

In certain embodiments the foam is an emollient foam formed from an oil-in-water emulsion comprising the halogenated salicylanilide. The oil may be, for example, a mineral oil, a plant derived oil (e.g. olive oil, soybean oil, coconut oil, or castor oil), medium or long-chain triglycerides and esters thereof, fatty acids, fatty acid esters, fatty acid alcohols and a wax. For example, the oil may comprise an alcohol selected from lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. The oil may comprise a fatty acid selected from dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid and pentatriacontanoic acid. The oil may comprise a hydroxy fatty acid such a 12-hydroxy stearic acid. The oil may comprise a wax, for example carnauba wax, candelilla wax, ouricury wax, sugarcane wax, retamo wax, jojoba oil, an animal wax (e.g. beeswax) or a petroleum derived wax (e.g. paraffin wax).

The emulsion may include emulsifiers or surfactants to stabilise the emulsion, for example one or more non-ionic surfactant (including any of the surfactants described herein, particularly those in relation to the non-aqueous topical compositions described above). The foam may comprise further excipients, for example, solvents, gelling agents, humectants, preservatives, and absorption enhancers, including but not limited to those described herein.

In a particular embodiment the foam is a non-aqueous foam. Such foams can be prepared by forming one of the non-aqueous formulations described above, for example a non-aqueous gel composition, into a foam composition. Examples of non-aqueous foam compositions which may be suitable for the delivery of a halogenated salicylanilide are described in, for example WO2010/041141, WO2009/098595 and WO2008/152444.

In certain embodiments the foams is a non-aqueous oil-based foam prepared using a suitable pharmaceutically acceptable oil, for example as discussed above in relation to emollient foams in which the halogenated salicylanilide is dispersed or dissolved. It may be that surfactants are used to stabilise the foams. It is also contemplated that non-aqueous oil-based foams may be prepared which do not require a surfactant. Such foams include but are not limited to those described in WO2011/013008, WO2011/013009, WO2011/064631 and WO2011/039637.

Other examples of foam compositions that may be used to formulate the halogenated salicylanilide include analogous compositions to those described in, for example, WO2011/138678, WO2011/039638, WO/2010/125470, WO/2009/090558, WO2009/090495, WO2009/007785, WO2008/038140, WO2007/085902, WO2007/054818, WO2007/039825, WO2006/003481, WO2005/018530, WO2005/011567 and WO2004/037225.

Foam compositions comprising the halogenated salicylanilide are suitably formulated as a semi-solid or liquid composition packaged in a suitable aerosol pressurised container with a propellant. The foam is formed upon release of the composition from the pressurised container via a suitable aerosol nozzle in the outlet of the container. Suitable propellants include a hydrocarbon propellant such as propane or butane, or a halogenated fluorocarbon such as tetrafluoroethane. Suitable aerosol containers and nozzles are well-known.

### Spot-on/Line on Compositions

In some embodiments the composition is formulated as a spot-on or line-on composition comprising the halogenated salicylanilide. Examples of spot-on or line-on compositions comprise the halogenated salicylanilide, or a pharmaceutically acceptable salt or hydrate thereof (e.g. niclosamide or oxyclozanide) and a solvent, preferably a non-aqueous solvent.

It may be that the spot-on or line on composition comprises the halogenated salicylanilide and one or more polar aprotic solvents. In some embodiments the polar aprotic solvent is selected from a ketone (e.g. acetone), N,N-dimethylformamide, acetonitrile, and dimethylsulfoxide (DMSO). Preferably the solvent is DMSO. In some embodiments the spot-on or line on compositions comprises one or more additional co-solvents. Suitably the co-solvent is a lipophilic solvent (for example an oil or, fat or lipid or any of the non-polar excipients described herein), a glycol described herein (e.g. PEG or propylene glycol), a glycol ether (e.g. 2-(2-ethoxyethoxy)ethanol), a protic polar solvent described herein, an alcohol, e.g. ethanol and/or an alkanol amine (e.g. ethanolamine, diethanolamine, triethanolamine, isopropanolamine and diisopropanolamine). The presence of a solvent in the spot-on or line-on composition enables the composition to be formulated with a high concentration of the halogenated salicylanilide, thereby enabling the “spot-on” or “line-on” of a concentrated solution or dispersion of the halogenated salicylanilide to the subject.

In some embodiments the spot-on or line-on composition comprises 2 to 20% wt/v, preferably 5 to 15% wt/v, more preferably, 8 to 12% wt/v of a halogenated salicynalinide (e.g. niclosamide or oxyclozanide); and
35 to 55% wt/v (preferably 30 to 50% more preferably, about 45%) of a polar aprotic solvent, for example dimethyl sulfoxide (DMSO).

Suitably the spot-on or line-on composition further comprises a glycol ether (e.g. 2-(2-ethoxyethoxy)ethanol) and optionally an alkanol amine (e.g. ethanolamine.

Accordingly in a preferred embodiment the spot-on or line on composition comprises:
2 to 20% wt/v, preferably 5 to 15% wt/v, more preferably, 8 to 12% wt/v of a halogenated salicynalinide (e.g niclosamide or oxyclozanide);
35 to 55% wt/v (preferably 30 to 50% more preferably, about 45%) of a polar aprotic solvent, for example dimethyl sulfoxide (DMSO);
25 to 55% w/v (preferably 30 to 55% wt/v, more preferably 35 to 50% w/v of a glycol ether (e.g. 2-(2-ethoxyethoxy)ethanol);
and 0 to 10% w/v (preferably 0 to 5% w/v (e.g. 0, or 1 to 5% w/v) alkanol amine (e.g. ethanolamine.

In another embodiment the spot-on or line-on composition is a composition selected from formulation A′ to I′ shown in Table A:

TABLE A

| | A′ | B′ | C′ | D′ | E′ | F′ | G′ | H′ | I′ |
|---|---|---|---|---|---|---|---|---|---|
| Oxyclozanide (wt/v %) | 8 | 9 | 10 | 12 | 15 | 20 | 2 | 5 | 11 |
| DMSO (wt/v %) | 40 | 42 | 46 | 45 | 45 | 46 | 40 | 40 | 45 |
| Monoethanolamine | 3 | — | — | — | — | 3 | 3 | — | — |
| Transcutol P (QSP) | Qsp | Qsp | Qsp | Qsp | Qsp | Qsp | Qsp | Qsp | Qsp |

A further aspect of the invention provides a spot-on or line-on composition as described herein.

### Optional Components for Topical Compositions

The following components and features may optionally be present in the halogenated salicylanilide compositions described herein, for example the non-aqueous topical compositions described herein.

### Solvents

The topical composition may comprise one or more solvent(s). The presence of a further solvent may enhance the solubility of the halogenated salicylanilide and or help maintain the halogenated salicylanilide in solution during the preparation, storage and topical use of the non-aqueous composition. The additional solvent may be, for example, a polar organic solvent in which the halogenated salicylanilide is soluble, for example a polar organic solvent wherein the halogenated salicylanilides has a solubility of greater than 2% by weight in the additional solvent.

The polar organic solvent may be a protic polar organic solvent. In one embodiment the solvent is a protic polar organic solvent having a dielectric constant of from about 10 to about 45, for example a dielectric constant of from about 10 to about 25. Particular polar protic organic solvents are those which have a dielectric constant of from about 10 to about 20, wherein in each case the dielectric constant is measured at 20-25° C. The dielectric constant of organic solvents is well known or can be measured using well-known techniques

Representative protic polar organic solvents with a dielectric constant in the range of 10 to 45 include those set out in the Table below:

| Solvent | Dielectric Constant at 20-25° C. |
|---|---|
| 2-methylpentane-2,4-diol (pinakon) | 7.4 |
| PEG 300 | 18.0 |
| PEG 400 | 14.1-12.4 |
| PEG 600 | 12.7 |
| N-octanol | 10.3 |
| Propylene glycol | 32 |
| Glycerol | 42.5 |
| Methanol | 33 |
| Ethanol | 24.3 |
| Propanol | 22 |

Further polar organic solvents with a dielectric constant in the range are well known (see for example “Solubility and Solubilization in Aqueous Media” By Samuel H. Yalkowsky (University of Arizona). Oxford University Press: New York. 1999). For example, the polar organic solvent may be selected from ethyl acetate, dimethylformamide, dichloromethane, glycerol, propylene glycol, or 2-(2-ethoxyethoxy)ethanol (Transcutol), propylene glycol stearyl ether and propylene glycol isostearate.

In embodiments the polar organic solvent is an aprotic polar organic solvent having a dielectric constant of from about 10 to about 45, for example a dielectric constant of from about 10 to about 25 at 25° C.

When present the additional solvent(s) is suitably present in an amount of up to 35% by weight of the composition. For example, up to 30%, 25%, 20% 15% or 10% by weight of the composition. In particular embodiments the additional solvent(s) is present in an amount of less than 10%, for example less than 8%, less than 6%, less than 5% or less than 3%, wherein the % is by weight based upon the weight of the non-aqueous composition. It may be that the additional solvent is present in an amount of 1% to 30%, from 1% to 25%, from 1% to 20%, from 1 to 10%, from 3 to 30%, from 3 to 20%, from 3 to 15%, from 5 to 30%, from, 5 to 20% or from 5 to 10%, wherein the % is by weight based upon the weight of the composition.

### Non-Ethanolic Compositions

The presence of ethanol in topical compositions can cause dryness and/or peeling of the skin, particularly in patients with sensitive skin. This can be a particular problem in patients with dermal conditions such as dermatitis (e.g. AD). Accordingly, in certain embodiments the topical composition comprising the halogenated salicylanilide is ethanol free. Thus, in a preferred embodiment the topical halogenated salicylanilide composition comprises a non-aqueous, non-ethanol (ethanol free) composition, for example a non-aqueous, non-ethanol gel composition.

### Absorption Enhancers

The topical composition may optionally comprise an absorption enhancer. The absorption may be any substance which acts to enhance the permeation of the halogenated salicylanilide into the epidermis and epidermis. Suitable absorption enhancers include the transdermal absorption enhancers disclosed in for example Smith and Maibach (2005) Percutaneous Penetration Enhancers, Second Edition ISBN 9780849321528, incorporated herein by reference.

It may be that the absorption enhancer, when present in the topical composition is selected from, for example, a sulfoxide (for example dimethylsulfoxide); dimethylacetamide; dimethylformamide; a urea; a fatty alcohol, for example a C₈-C₁₈ fatty alcohol, which may be saturated or unsaturated (for example caprylic alcohol or cetostearyl alcohol); a polyol (for example glycerol; a glycol (for example propylene glycol or hexylene glycol); Azone ((1-dodecylazacycloheptan-2-one); an essential oil (for example a terpene or terpenoid); a pyrrolidone (for example N-methyl-2-pyrrolidone); an oxazolidinone (for example 4-decyloxazolidin-2-one) a surfactant (for example a non-ionic, anionic or cationic surfactant, particularly a non-ionic surfactant for example a polyoxyethylene glycol sorbitan alkyl ester (for example polysorbates such as Polysorbate 80 ((polyoxyethylene (20) sorbitan monooleate), Polysorbate 60 (polyoxyethylene (20) sorbitan monostearate), Polysorbate 40 (polyoxyethylene (20) sorbitan monopalmitate) or Polysorbate 20 (polyoxyethylene (20) sorbitan monolaurate)), a polyoxyethylene glycol alkyl ether (Brij surfactants e.g. polyethoxylated stearyl ethers such as Brij S721 (a polyoxyethylene fatty ether derived from stearyl alcohols) or Brij S2 (Polyoxyethylene (2) stearyl ether)), a poloxamer or a PEGylated fatty acid glyceride such as caprylocaproyl polyoxyl-8 glycerides (e.g. Labrasol), a fatty acid ester of glycerol, for example glyceryl stearate, or polyoxyethylene ethers of fatty alcohols (for example cetyl alcohol and/or stearyl alcohol, particular examples include ceteareth-15, -16, -17, -18, -19, -20, -21, -22, 23-, -24, or -25 and particularly ceteareth-20), a polyethoxylated sorbitan fatty acid ester, for example. The absorption enhancer may also be 2-(2-ethoxyethoxy)ethanol (Transcutol). Preferred absorption enhancers are those which have a minimal impact on the structure of the skin so as to minimise undesirable tolerability effects associated with the absorption enhancer, for example irritation, which could exacerbate the dermatitis (e.g. AD) in the subject. Particular absorption enhancers include polyols, for example propylene glycol or glycerol. Accordingly the absorption enhancer may be propylene glycol. The absorption enhancer may be glycerol. It is to be understood that where the absorption enhancer may also act as an additional solvent in the composition, particularly when the halogenated salicylanilide is soluble in the absorption enhancer.

When present the absorption enhancer may be in an amount of up to 35% by weight of the topical composition (e.g. a gel composition), for example from 0.5% to 35%, from 1% to 35%, from 5% to 30%, from 10% to 30%, from 5% to 35%, from 5% to 30% or from 10% to 30%, wherein the % is by weight of the composition.

### Other Ingredients

The halogenated salicylanilide compositions described herein (e.g. a topical composition) may comprise one or more additional excipients in addition to the halogenated salicylanilide and the other excipients described above (e.g. PEG in a non-aqueous topical composition). Additional excipients may be selected to provide compositions of the required form for topical administration. The additional excipients may be, for example one or more excipients selected from viscosity modifying agents, emulsifiers, surfactants, humectants, oils, waxes, solvents, preservatives, pH modifying agents (for example a suitable acid or base, for example an organic acid or organic amine base), buffers, antioxidants (for example butylated hydroxyanisol or butylated hydroxytoluene), crystallisation inhibitors (for example a cellulose derivative such as hydroxypropylmethyl cellulose), colorants, fragrances. Representative examples of such additional excipients are well known, for example as listed in the Handbook of Pharmaceutical Excipients, 7^{th} Edition, Rowe et al. Further more specific excipients are set out in any of the non-aqueous compositions described in the Examples herein.

### Certain Embodiments

In some embodiments the composition is not a non-aqueous topical composition comprising a halogenated salicylanilide selected from niclosamide, rafoxanide, oxyclozanide and closantel, or a pharmaceutically acceptable salt or hydrate thereof; and polyethylene glycol.

In certain embodiments the topical composition comprising the halogenated salicylanilide (e.g. niclosamide or oxyclozanide) does not contain DMSO.

In certain embodiments the composition comprising the halogenated salicylanilide (e.g. niclosamide or oxyclozanide) is not one of the compositions disclosed in WO 2019/053180.

In certain embodiments the composition comprising the halogenated salicylanilide (e.g. niclosamide or oxyclozanide) is not Composition W, Composition X or Composition Y:

Composition W: a topical veterinary spot on or line on composition comprising 2 to 20 wt/v % of at least one halogenated salicylanilide, or a pharmaceutically acceptable salt or hydrate thereof, 35 to 55 wt/v % dimethyl sulfoxide, wherein the at least one halogenated salicylanilide is selected from niclosamide and/or oxyclozanide and wherein the composition is dissolved in diethylene glycol monomethyl ether.

Composition X: a topical veterinary spot on or line on composition comprising 2 to 20 wt/v % of at least one halogenated salicylanilide, or a pharmaceutically acceptable salt or hydrate thereof, 35 to 55 wt/v % dimethyl sulfoxide, wherein the at least one halogenated salicylanilide is selected from niclosamide and/or oxyclozanide and wherein the composition is dissolved in diethylene glycol monoethyl ether (Transcutol).

Composition Y: a topical composition selected from Table A above.

In certain embodiments the composition is not Composition W, X, or Y, for use in the topical treatment or prevention of pyoderma or dermatitis in a non-human mammal.

In certain embodiments the composition is not Composition W, X, or Y, for use in the treatment or prevention of pyoderma or dermatitis in a non-human mammal wherein the composition is topically applied to the non-human mammal as a single application optionally repeated a number of times every 5 to 10 days, for example once every 5 to 10 days for 3 to 5 consecutive weeks.

### Manufacture of Topical Compositions

The topical compositions described herein may be manufactured using well-known methods. For example, the non-aqueous gel compositions comprising PEG may be prepared by a process comprising the steps:
(i) dissolving the halogenated salicylanilide in the PEG;
(ii) combining the solution from step (i) with the gel-forming agent to form a mixture; and
(iii) causing the mixture to gel.

Suitably the halogenated salicylanilide is completely dissolved in the PEG in step (i) to form a solution. Dissolution may be aided by agitation of the mixture by stirring or by the application ultrasound. Optionally the mixture may be heated to facilitate dissolution. However, preferably the solution is prepared at ambient temperature. Optionally any halogenated salicylanilide that remains undissolved may be removed by a suitable filtration or other separation method prior to combining the solution with the gel-forming agent in step (ii) of the process.

The solution from step (i) may be added to the gel-forming agent or, alternatively, the gel-forming agent may be added to the solution. Optionally the gel-forming agent may be dissolved in some of the PEG to form a solution or dispersion prior to combining it with the solution from step (i). Suitably any additional optional components of the gel-composition, such as absorption enhancers, additional solvents etc. are added to the mixture prior to gelation of the composition. Alternatively, one or more of the optional components can be added after gel formation by mixing the additional component(s) with the gel.

Gel formation in step (iii) may be affected by various methods, depending on the nature of the gel-forming agent used. For example, where the gel-forming agent is thermotropic, the gel forming agent may be heated to form a liquid prior to adding the solution from step (i). Following mixing of the gel-forming agent with the solution, the resulting mixture may be cooled thereby causing the mixture to gel. Alternatively, where gelling is effected by ionic cross-linking, a suitable ionic agent is added to the mixture in step (iii), for example a suitable salt to thereby cause the mixture to gel. Gelling may also be induced by changing the pH of the mixture using a suitable acid or base to achieve the required pH for gelling to occur. The process is suitably carried out using anhydrous reagents under anhydrous conditions to ensure that the resulting gel composition is a non-aqueous gel composition.

When the gel-forming agent is a carbomer, a particular process for the preparation of the non-aqueous gel composition comprises:
(i) dissolving the halogenated salicylanilide in the PEG;
(ii) combining the solution from step (i) with a carbomer to form a mixture; and
(iii) heating the mixture to form a gel.

Step (i) of this process is suitably performed at room temperature. After combining the solution with the carbomer the mixture is mixed to provide a uniform dispersion. Mixing can be performed using any suitable method, for example stirring or, preferably, by homogenisation. The resulting dispersion is suitably de-gassed prior to gel formation in step (iii).

In step (iii) the mixture is suitably heated to a temperature of 60 to 80° C., for example at about 70° C., preferably under agitation. The mixture may be held at this temperature for a sufficient time to form a homogenous and transparent dispersion and to effect gel formation. Typically a holding time of about 30 minutes is sufficient to enable solvation of the carbomer and gel formation.

The process is suitably performed under anhydrous conditions using anhydrous reagents to ensure that the resulting gel composition is a non-aqueous gel.

When the composition of the invention is in the form of a lotion, ointment or cream the composition may be prepared using known methods for the preparation of such compositions. For example, lotion or ointments may be prepared by simply blending the halogenated salicylanilide, and the other excipients comprising the formulation, for example viscosity modifiers, solvents and/or surfactants.

Non-aqueous topical compositions may also be prepared as non-aqueous emulsion or microemulsions to provide a composition in the form of, for example a non-aqueous cream. Non-aqueous emulsions and microemulsions may be prepared using well known methods. Non-aqueous emulsions and microemulsions may be prepared by mixing two immiscible non-aqueous phases. Suitably a non-aqueous hydrophilic phase (for example a hydrophilic phase comprising polar excipients and the halogenated salicylanilide) is emulsified with an immiscible hydrophobic phase (e.g. comprising non-polar hydrophobic excipients). The non-aqueous emulsion may comprise a continuous hydrophobic phase and a discontinuous hydrophilic phase. Generally however, the non-aqueous emulsion will comprise a continuous hydrophilic phase and a discontinuous hydrophobic phase. It may be that the non-aqueous hydrophilic phase comprises the halogenated salicylanilide and PEG and the non-aqueous hydrophobic phase comprises a non-polar liquid, which is immiscible with the hydrophobic phase, for example a medium chain triglyceride, a vegetable oil, a hydrocarbon oil or a mineral oil such as a paraffin. Generally the non-aqueous emulsion will be stabilised by one or more suitable surfactants or emulsifiers, for example one or more non-ionic surfactants (e.g. macrogol cetostearyl, cetostearyl alcohol, glyceryl stearate, polysorbate 80, Brij s721, Brij S2, ceteareth-20 or macrogol stearyl ether). The emulsion or micro emulsion may be formed using well-known methods, for example by homogenisation of the hydrophilic phase with the hydrophobic phase together with the other components of the non-aqueous emulsion or microemulsion.

### Dosages and Dosage Regimens

An effective amount of the halogenated salicylanilide for use in the treatment of dermatitis (e.g. AD) is an amount sufficient to relieve the non-human subject of one or more of the symptoms of dermatitis (e.g. AD) described herein or to slow the progression or development of dermatitis (e.g. AD).

The amount of active ingredient that is combined with one or more excipients to produce a single dosage form will necessarily vary depending upon the host treated and the particular route of administration. For example, a formulation intended for topical administration to an animal will generally be administered in an amount sufficient to cover the dermatitis lesion. Suitably the composition is applied in an amount to provide a dose of the halogenated salicylanilide of from about 0.001 to about 1 mg/cm²; about 0.01 to about 0.5 mg/cm²; about 0.01 to about 0.5 mg/cm²; or about 0.01 to about 0.3 mg/cm², for example about 0.04, 0.05, 0.06, 0.07, 0.08, 0.09, 1, 1.1, 1.2, 1.3, 1.4, 1.5, 1.6, 1.7, 1.8, 1.9, 2.0, 2.1, 2.2, 2.3, 2.4 or 2.5 mg/cm². In some embodiments the halogenated salicylanilide (e.g. oxyclozanide) is topically administered such that each topical administration of the halogenated salicylanilide is in a dose of from 0.1 mg·kg to 100 mg/kg (e.g. from 1 to 50 mg/kg, from 1 to 40 mg/kg, from 1 to 30 mg/kg, about 1 mg/kg, about 5 mg/kg, about 10 mg/kg, about 20 mg/kg or about 25 mg/kg). The composition will be applied in an amount sufficient to provide this desired dose of the halogenated salicylanilide. This will of course depend on the concentration of the halogenated salicylanilide in the composition. Typically the composition will be applied in an amount of about 0.1 to about 50 mg/cm²; about 1 to about 20 mg/cm²; about 1 to about 5 mg/cm²; about 2 to 5 mg/cm²; about 2 to about 15 mg/cm² or about 4 to about 10 mg/cm².

In some embodiments the halogenated salicylanilide is topically administered to the non-human subject is a dose of 0.5 to 5 ml per 10 kg of body weight, preferably, 1 to 3 ml per 10 Kg of body weight, even more preferably, about 2 ml per 10 Kg of body weight of the subject. For example in this embodiment the halogenated salicylanilide is administered as a spot on/pour on/line on composition (for example a spot-on, line-on or pour-on composition described herein comprising the halogenated salicylanilide at a concentration of e.g 2 to 20% wt/v, preferably 5 to 15% wt/v, more preferably, 8 to 12% wt/v). The composition is suitably formulated as a unit dose adapted to the weight and/or size of the non-human mammal. Preferably the entire dose is topically applied to the animal as a single “spot” or “line

When administered topically to the non-human subject, the halogenated salicylanilide is suitably applied directly to a dermatitis lesion. Suitably the halogenated salicylanilide is topically applied in the form of a topical composition and is gently rubbed into the skin at the site of the lesion to be treated so as to provide coverage of substantially all of the lesion. Optionally a composition comprising the halogenated salicylanilide may be topically applied using a suitable carrier substrate, for example a wound dressing or a patch impregnated with or carrying a composition comprising the halogenated salicylanilide. The carrier may be applied to a lesion such that the lesion is brought into contact with the halogenated salicylanilide present in or on the carrier substrate.

The frequency of (e.g. topical) administration of the halogenated salicylanilide depend upon a number of factors that may readily be determined by a physician, for example the severity of the dermatitis (e.g. AD). Suitably the halogenated salicylanilide is topically administered 1, 2, 3 or 4 times per day. The duration of the treatment may be, for example, 1 week or more, 2 weeks or more, 3 weeks or more, 4 weeks or more, 6 weeks or more, 12 weeks or more, 6 months or more, or 1 year or more.

In some embodiments the mean plasma Cₘₐₓ of the halogenated salicylanilide or after topical application of the halogenated salicylanilide is less than about 2000 μg/l, 1500 μg/l, for example less than 1000 μg/l, less than 500 μg/l, or less than 200 μg/l. The plasma Cₘₐₓ will vary depending on the dose of the halogenated salicylanilide topically administered, the area of the skin to which the compound is topically applied and possibly the dosing frequency. In some embodiments the mean plasma Cₘₐₓ of the halogenated salicylanilide is less than about 2000 μg/l, 1500 μg/l, for example less than 1000 μg/l, less than 500 μg/l, or less than 200 μg/l, wherein the halogenated salicylanilide is topically administered as a single dose of 20 mg/kg, or a mean Cₘₐₓ directly proportional thereto for a topically applied dose other than 20 mg/kg. In some embodiments the mean plasma Cₘₐₓ of the halogenated salicylanilide (e.g. oxyclozanide), is less than about 1500 μg/l, for example less than 1000 μg/l, less than 500 μg/l, or less than 200 μg/l, when topically administered as a single dose of 20 mg/kg applied as a line along the spine of an animal such as a dog.

In some embodiments the mean plasma concentration of the halogenated salicylanilide (e.g. oxyclozanide) measured over a period of 24 to 96 hours after topically administering a single dose of 20 mg/kg of the halogenated salicylanilide applied as a line along the spine of an animal such as a dog is less than about 1300 μg/l, for example less than about 800 μg/l, less than 700 μg/l, less than 500 μg/l, less than 200 μg/l, or less than 150 μg/l, or a concentration directly proportional thereto for a single dose other than 20 mg/kg. For example, the mean plasma concentration of the halogenated salicylanilide (e.g. oxyclozanide) in the period of 24 to 96 hours after topical administration of the halogenated salicylanilide (e.g. oxyclozanide) may be from about 20 to about 200 μg/l or about 50 μg/l to about 150 μg/l.

The plasma Cₘₐₓ in the embodiments described herein is suitably determined in the period after the last topical administration of the halogenated salicylanilide of the initial treatment period. The Cₘₐₓ may be determined by taking regular blood samples after the last dose of the halogenated salicylanilide so as to determine the maximum plasma concentration. Generally, samples taken once per hour or once every 2 hours for 12 hours after the last topical administration of the halogenated salicylanilide will be sufficient to determine the Cₘₐₓ value. The plasma concentration of the halogenated salicylanilide (e.g. oxyclozanide), for example to determine the mean plasma concentration in the period of 24 to 96 hours after topical administration of the halogenated salicylanilide, may be measured using well-known methods.

In some embodiments the mean Cₘₐₓ of the halogenated salicylanilide (e.g. oxyclozanide) in the stratum corneum measured over a period of 1 day to 28 days after topically administering a single dose of 20 mg/kg of the halogenated salicylanilide (e.g. oxyclozanide) applied as a line along the spine of an animal such as a dog is greater than 50 μg/g, or a concentration directly proportional thereto for a single dose other than 20 mg/kg. For example, the mean Cₘₐₓ in the stratum corneum after topically administering a single dose of 20 mg/kg of the halogenated salicylanilide (e.g. oxyclozanide) is greater than about 60 μg/g, about 70 μg/g, about 80 μg/g, about 90 μg/g, about 100 μg/g, about 110 μg/g, about 120 μg/g, about 150 μg/g, about 175 μg/g or about 200 μg/g, or a concentration directly proportional thereto for a single dose other than 20 mg/kg. It may be that the mean Cₘₐₓ in the stratum corneum after topically administering a single dose of 20 mg/kg of the halogenated salicylanilide (e.g. oxyclozanide) is from about 30 to about 700 μg/g, from about 50 to about 600 μg/g, from about 60 to about 550 μg/g, or from about 60 to about 500 μg/g, or a concentration directly proportional thereto for a single dose other than 20 mg/kg.

In some embodiments the mean Cₘₐₓ of the halogenated salicylanilide (e.g. oxyclozanide) in the dermis and epidermis measured over a period of 1 day to 28 days after topically administering a single dose of 20 mg/kg of the halogenated salicylanilide (e.g. oxyclozanide) applied as a line along the spine of an animal such as a dog is greater than 1 μg/g, or a concentration directly proportional thereto for a single dose other than 20 mg/kg. For example, the Cₘₐₓ in the dermis and epidermis after topically administering a single dose of 20 mg/kg of the halogenated salicylanilide (e.g. oxyclozanide) is greater than about 1.5 μg/g, about 2 μg/g, about 2.5 μg/g, about 3 μg/g, about 3.5 μg/g, about 4 μg/g, about 4.5 μg/g or about 5 μg/g, or a concentration directly proportional thereto for a single dose other than 20 mg/kg. It may be that the Cₘₐₓ in the dermis and epidermis after topically administering a single dose of 20 mg/kg of the halogenated salicylanilide (e.g. oxyclozanide) is from about 1 to about 13 μg/g, from about 1.5 to about 10 μg/g or from about 2 to about 8 μg/g, or a concentration directly proportional thereto for a single dose other than 20 mg/kg.

In some embodiments the area under the curve of the halogenated salicylanilide (e.g. oxyclozanide) in the stratum corneum (AUC₂₈) 28 days after topically administering a single dose of 20 mg/kg of the halogenated salicylanilide (e.g. oxyclozanide) applied as a line along the spine of an animal such as a dog is greater than 500 day*μg/g, or a value directly proportional thereto for a single dose other than 20 mg/kg. For example the AUC₂₈ is greater than 600 day*μg/g, greater than 700 day*μg/g, greater than 800 day*μg/g, greater than 900 day*μg/g, greater than 1000 day*μg/g, greater than 1200 day*μg/g or greater than 1500 day*μg/g. It may be that the AUC₂₈ is from 800 day*μg/g to 7000 day*μg/g, from 850 day*μg/g to 6000 day*μg/g, or from 850 day*μg/g to 5500 day*μg/g.

In some embodiments, the time to reach maximum concentration of the halogenated salicylanilide (e.g. oxyclozanide) in the skin, Tₘₐₓ, (e.g. in the stratum corneum, or the dermis and epidermis) at sites remote from the point of application after topically administering a single dose of, for example, 20 mg/kg of the halogenated salicylanilide (e.g. oxyclozanide) applied as a line along the spine of an animal such as a dog, is from about 1 to 10 days, for example from 2 to 10 days, from 3 to 8 days or from 4 to 8 days. The site remote from the point of topical application of the halogenated salicylanilide may be, for example the belly, chest, ear, fore leg, hind leg or shoulder of the animal (e.g. dog).

The concentration of the halogenated salicylanilide in the skin (or individual skin layers) may be assessed by measuring the concentration in a skin biopsy taken from the subject following topical administration of the halogenated salicylanilide, for example using the methods described herein and in the Examples (e.g. a tape stripping method). The Cₘₐₓ, AUC and Tₘₐₓ values may be calculated using well known methods based on the measured concentrations of the halogenated salicylanilide in the skin sample, such methods are illustrated in the Examples herein.

### Subjects

The subject topically treated with the halogenated salicylanilide is a non-human subject. The subject may be a warm blooded non-human mammal. In some embodiments the subject is a commercial animal such as livestock (e.g. cows, sheep, chickens, pigs, geese, ducks, goats, etc.). In other embodiments subject is a companion animal such as a cat, dog or horse. In some embodiments the subject is a dog or a cat. In a particular embodiment the subject is a dog.

In some embodiments the subject is a dog and the dermatitis is selected from canine atopic dermatitis, flea allergy dermatitis, scabies, malassezia dermatitis, intertrigo, pododermatitis, demodicosis, contact dermatitis and canine bacterial pyoderma.

In some embodiments the subject is a cat and the dermatits is selected from flea allergy dermatitis, atopic dermatitis, food allergic dermatitis otodectic acariasis and feline bacterial pyoderma.

### EXAMPLES

### Example 1: Non Aqueous Topical Niclosamide Formulations

### Non Aqueous Topical Niclosamide Gel Formulation

The topical gel compositions shown in Table 1 were prepared:

TABLE 1

| | Composition | |
|---|---|---|
| Raw material INCI or PhEur name | Formulation A | Formulation B |
| (trade name) | % (w/w) | % (w/w) |
| Niclosamide, anhydrous | 2.0 | 4.0 |
| Macrogol 400 | 95.6 | 93.6 |
| Carbomer 974P (Carbopol 974P) | 2.4 | 24 |

The composition was prepared as follows. Niclosamide 200 mg, PEG 400 (9.56 g for Formulation A and 9.36 g for Formulation B) were weighed in blue cap bottles. The mixture was stirred at room temperature until a clear solution formed. 240 mg. Carbomer 974P was then dispersed in the niclosamide PEG 400 solution. The dispersion was homogenized and degassed. The suspension was then heated at 70° C. and stirred mechanically at 250 rpm until a homogeneous dispersion formed after about 30 minutes. The final solution was then cooled to give the title non-aqueous gel compositions.

The final formulations were protected from light prior to further use.

Further Non-Aqueous Topical compositions

The non-aqueous topical compositions shown in Tables 2 and 3 were prepared

TABLE 2

| | Composition | | | |
|---|---|---|---|---|
| | Formulation D | Formulation E | Formulation F | Formulation G |
| | Appearance | | | |
| | | Shiny rather | Very shiny | Slightly shiny |
| | | hard yellow | hard yellow | hard yellow |
| | Very shiny, | ointment, | ointment, | ointment, |
| | soft yellow | becomes soft | becomes soft | becomes soft |
| Raw material INCI or | ointment | upon shearing | upon shearing | upon shearing |
| PhEur name (trade name) | % (w/w) | % (w/w) | % (w/w) | % (w/w) |
| Active | | | | |
| Niclosamide anhydrous | 2.0 | 2.0 | 2.0 | 2.0 |
| Hydrophilic Phase | | | | |
| Macrogol 400 | 30.0 | 20.0 | 27.5 | 30.0 |
| Propylene glycol | 25.0 | 15.0 | 17.5 | 15.0 |
| Ethoxydiglycol (Transcutol) | | 15.0 | 5.0 | 15.0 |
| Glycerol | 19.7 | | | |
| Hydroxyethyl cellulose | 0.25 | | | |
| (Natrosol 250G Pharm) | | | | |
| Hydrophobic Phase | | | | |
| and emulsifiers | | | | |
| Polysorbate 80 | | 1.0 | 1.0 | 1.0 |
| Ceteareth-20 | 6.0 | | | |
| (Cetomacrogol 1000-PA) | | | | |
| Caprylocaproyl Macrogol | 10.0 | | | |
| 8 glycerides (Labrasol) | | | | |
| Steareth-21 (Brij S721) | | 5.0 | 5.0 | 5.0 |
| Steareth-2 (Brij S2) | | 5.0 | 5.0 | 5.0 |
| Macrogol stearyl ether | | | 5.0 | |
| (Arlamol PS11E) | | | | |
| Paraffin, liquid | | 10.5 | 5.5 | 6.5 |
| Medium chain triglycerides | | 6.0 | 6.0 | |
| Paraffin, Type 5205, hard | | 6.5 | 6.5 | 6.5 |
| Cetostearyl alcohol | 7.0 | 12.0 | 12.0 | 12.0 |
| (Kolliwax CSA 50) | | | | |
| Glyceryl stearate, Type II | | 2.0 | 2.0 | 2.0 |
| (Kolliwax GMS II) | | | | |

TABLE 3

| | Composition | | |
|---|---|---|---|
| | Formulation H | Formulation I | Formulation J |
| | Appearance | | |
| | Shiny rather hard | | |
| | yellow ointment, | Very shiny very soft | Soft shiny |
| | becomes soft upon | yellow ointment, | yellow ointment, |
| | shearing, liquefies | becomes softer | becomes softer |
| Raw material INCI or | at skin temp. | upon shearing | upon shearing |
| PhEur name (trade name) | % (w/w) | % (w/w) | % (w/w) |
| Active | | | |
| Niclosamide, anhydrous | 2.0 | 0.5 | 0.5 |
| Hydrophilic Phase | | | |
| Macrogol 400 | 10.0 | 20.0 | 10.0 |
| Propylene glycol | 20.0 | 10.0 | 20.0 |
| Ethoxydiglycol (Transcutol) | 15.0 | 15.0 | 15.0 |
| Hydrophobic Phase and | | | |
| emulsifiers | | | |
| Polysorbate 80 | 1.0 | 1.0 | 1.0 |
| Steareth-21 (Brij S721) | 5.0 | 5.0 | 5.0 |
| Steareth-2 (Brij S2) | 5.0 | 5.0 | 5.0 |
| Paraffin, liquid | 11.5 | 14.5 | 14.5 |
| Medium chain triglycerides | 10.0 | 10.0 | 10.0 |
| Paraffin, Type 5205, hard | 6.5 | 5.0 | 5.0 |
| Cetostearyl alcohol | 12.0 | 12.0 | 12.0 |
| (Kolliwax CSA 50) | | | |
| Glyceryl stearate, Type II | 2.0 | 2.0 | 2.0 |
| (Kolliwax GMS II) | | | |
| Carbomer 974P ( Carbopol 974P) | | | |

The ointment formulations D, E, F, G, H, I and J set out in Tables 2 and 3 were prepared as non-aqueous emulsions using the following general method.

The hydrophilic phase of the emulsion and the anhydrous niclosamide (see under heading “hydrophilic phase” in Tables 2 and 3) were mixed together with stirring in a vessel to form a solution of the niclosamide in the hydrophilic phase. Generally the hydrophilic phase was heated gently at a temperature of about 60 to 75° C. (generally at about 70° C.) to aid dissolution of the niclosamide.

A hydrophobic phase comprising the oils and emulsifiers under the heading “Hydrophobic phase and emulsifiers” were mixed together by stirring in a heated vessel. The temperature was about 60 to 75° C. (generally at about 70° C.).

The hydrophobic phase and the hydrophilic phases were mixed together with gentle stirring so as to avoid phase separation and the mixture was cooled to a temperature of about 40 to 50° C. The mixture was then homogenised to give the final composition.

The appearance and some of the properties of the resulting compositions is described in the row marked “Appearance” in Tables 2 and 3.

### Spot-on or Line-on Formulations

Compositions A′ to I′ shown in Table B below were prepared:

TABLE B

| | A′ | B′ | C′ | D′ | E′ | F′ | G′ | H′ | I′ |
|---|---|---|---|---|---|---|---|---|---|
| Oxyclozanide (wt/v %) | 8 | 9 | 10 | 12 | 15 | 20 | 2 | 5 | 11 |
| DMSO (wt/v %) | 40 | 42 | 46 | 45 | 45 | 46 | 40 | 40 | 45 |
| Monoethanolamine | 3 | — | — | — | — | 3 | 3 | — | — |
| Transcutol P (QSP) | Qsp | Qsp | Qsp | Qsp | Qsp | Qsp | Qsp | Qsp | Qsp |

Transcutol P is diethylene glycol monomethyl ether also known as 2-(2-ethoxyethoxy)ethanol.

The compositions may be prepared be dispersing the oxyclozanide into the DMSO, Transcutol and optional monoethanolamine with stirring to form a solution.

### Example 2: Clinical Trial to Assess the Safety and Efficacy of Topically Applied Niclosamide in Healthy Volunteers

### Study Design

A prospective, single centre, randomized, double-blind, Placebo controlled study.

### Primary Objective of the Trial

The primary objective of the study is to demonstrate the safety and tolerability of topical niclosamide formulations in healthy volunteers.

### Secondary Objectives of the Trial:

To determine the local and systemic exposure of the topical niclosamide composition.

### Exploratory Objective:

To collect illustrative information on local tolerability of the topical niclosamide composition.
To determine the best tolerated formulation to advance into Phase II of the trial.

### Patients:

Randomization ratio 1:1; randomized niclosamide composition or Placebo application on right or left arm.

Inclusion criteria:
Signed and dated informed consent has been obtained.
Age 18-70 years.
Male or female.
Female subjects of childbearing potential must be confirmed not pregnant by a negative urine pregnancy test prior trial treatment.
Female subjects of childbearing potential must be willing to use effective contraceptive at trial entry until completion.
Male subjects must agree to use adequate contraception for the duration of the trial.

### Exclusion Criteria:

Regular use of medications unless considered clinically irrelevant by the Investigator.
Use of any dermatological drug therapy on the arms within 14 days before day 1 of this study.

### Protocol

The study comprised a group with 30 healthy volunteers. Each of these volunteers were treated in four separate areas two times daily with the niclosamide topical formulations or the vehicle controls during a seven-day period.

The following topical niclosamide formulations were tested:
2% niclosamide non-aqueous dermal gel: Formulation A described in Table 1 above
2% niclosamide non-aqueous dermal cream: Formulation G described in Table 2 above
   For each arm of the trial a placebo formulation comprising the vehicle only (i.e. without the niclosamide) was also tested.

### Dosage and Administration

Route of administration: topical.
Duration of treatment: 7 days.

Each volunteer had 4 formulations (2 active formulations and their respective Placebos) applied to defined skin areas in the dorsal arms. The body area to be treated was a circle marked by a skin marker with a diameter of 5 cm (approx. 20 cm²). The healthy volunteers had the body areas treated two times per day, at 08:00 (+/−2 hours) and 20:00 (+/−2 hours), respectively for 7 days. The expected dose of each formulation was 2 to 5 mg of product/cm²/day (corresponding to 0.04-0.1 mg niclosamide/cm²). The dermal formulation was left to dry for 10 minutes after application.

A screening visit was performed at Day 0. At Day 1 the patients were randomized, and this was also be the first day of treatment. On days 1-7 the healthy volunteers were treated twice daily at the study site. On Day 8 a final dose was applied in conjunction with the PK analysis. A final examination (end of study) was made on Day 15.

Six additional healthy volunteers were also enrolled for method testing. Treatments were blinded to both volunteers and doctors. The body area to be treated was be a circle with a diameter of approximately 5 cm and the expected dose of 2-5 mg of product/cm² (0.04-0.1 mg active substance per cm²).

The healthy volunteers in the trial were also subjected to a PK analysis after the last dose. The PK analysis involved sampling of blood after the final exposure to assess systemic exposure to niclosamide and skin biopsy sampling to assess local exposure to niclosamide in the skin. The 30 healthy volunteers were randomized for single punch biopsies to collect 10 biopsy samples from each active formulation. This meant that 1 active treatment area for each healthy volunteer had to be unblinded prior to biopsy sampling. To ensure that this did not interfere with the blind assessment of the safety of the formulations, safety was assessed in the morning of day 8, then on day 8 a 15th dose was given in conjunction with the bioanalysis. Biopsies were taken 1 h (+/−10 min) after application of the respective formulation.

### Punch Biopsies

The skin biopsies were taken using sterile single use disposable biopsy punches (BP40F, Kai Europe GmbH, Solingen, Germany). For the 6 non-treated healthy volunteers biopsies were taken on Day 1. 10 mL of blood was collected at day 1 for the method validation group to determine niclosamide concentration in the blood.

For the 30 treated healthy volunteers skin biopsies were taken on day 8 one hour after the 15^{th} application (+/−10 minutes).

The concentration of niclosamide in the skin biopsy samples was determined using validated bioanalytical UPLC-MS/MS methods.

The following chromatographic conditions were used:

| Parameter | Value | |
|---|---|---|
| Column | ZIC-cHILIC, 3 μm, 100 Å, | |
| | PEEK 100 × 2.1 mm, HX56413157 | |
| Column Temperature | 40° | |
| Mobile phase | A: 20 mM ammonium formate (pH = 3.5) | |
| | B: CH₃CN | |
| Mobile Phase Flow Rate | 0.4 ml/min, gradient mode | |
| Gradient Program | Time (min) | Mobile phase B % |
| | 0.0 | 50 |
| | 1.0 | 50 |
| | 2.0 | 70 |
| | 2.2 | 50 |
| | 3.5 | 50 |

| | |
|---|---|
| Injection volume | 1 μl |
| Autosampler Temperature | 4° |
| Time of analysis | 3.5 min |
| Detection | MRM |

Mass spectrometry was performed using a Shimadzu 8050 mass spectrometer operating in Electrospray negative mode (ESI^{-ve}).

### Preparation of Skin Biopsy Samples Containing Niclosamide

Extraction of skin biopsy samples was performed as follows:
1. Cut the tissue into small pieces and extract with 5.0 ml of DMSO/acetonitrile (50/50 v/v) at room temperature overnight using a shaker.
2. Spin down the tissue at 3700 g, collect supernatant and store it in a freezer (−20° C.).

### Determination of Niclosamide Concentration in Skin Biopsies

50 μl of untreated human skin extract was spiked with 10 μl of working standard solution (concentration of standard will be provided for each parameter). Samples were vortexed, then 200 μl of methanol/water solution 1;1 (v:v) was added. Finally, samples were centrifuged for 10 minutes at 4° C. at 2000 g. The supernatant was transferred into HPLC plate and analysed using UPLC-MS/MS.

### Assessment of Local Tolerability

Local dermal tolerability at the sites of application of the topical formulations was assessed by the investigator at all treatment visits using an 8-point dermal assessment score, in accordance with the FDA guideline on Skin Irritation and Sensitization Testing (1999). A dermal assessment score of 0 to 7 was defined as follows:
0=no evidence of irritation,
1=minimal erythema, barely perceptible,
2=definite erythema, readily visible; minimal edema or minimal papular response,
3=erythema and papule,
4=definite edema,
5=erythema, edema, and papule,
6=vesicular eruption,
7=strong reaction spreading beyond test site.
   Results from the Trial

All of the topical dermal niclosamide formulations and placebo formulations were well tolerated with no signs of adverse reactions at the sites of administration. All 6 investigational medicinal products were scored at 0 in all subjects at all time points, see Table 4.

TABLE 4
Mean Local Tolerability Scores at Treatment Visits

| | Mean Local Tolerance Score | | | | | | |
|---|---|---|---|---|---|---|---|
| Formulation | Day 2 | Day 3 | Day 4 | Day 5 | Day 6 | Day 7 | Day 8 |
| A: niclosamide GEL 2% | 0.0 | 0.0 | 0.0 | 0.0 | 0.0 | 0.0 | 0.0 |
| A: niclosamide GEL Placebo | 0.0 | 0.0 | 0.0 | 0.0 | 0.0 | 0.0 | 0.0 |
| G: niclosamide CREAM 2% | 0.0 | 0.0 | 0.0 | 0.0 | 0.0 | 0.0 | 0.0 |
| G: niclosamide CREAM Placebo | 0.0 | 0.0 | 0.0 | 0.0 | 0.0 | 0.0 | 0.0 |

Systemic exposure to niclosamide was minimal with mean serum concentration of niclosamide 0.24 ng/mL, while local exposure to the skin was substantial (see Table 5).

TABLE 5
Niclosamide Concentration in Skin Biopsies

| | | Anhydrous |
|---|---|---|
| | GEL 2% | CREAM 2% |
| | Formulation A | Formulation G |
| μg/g niclosamide | 5.8 ± 4.3 | 7.6 ± 4.9 |

Results from Phase One of the study showed that the dermal niclosamide and placebo formulations tested were well tolerated locally with no signs of adverse reactions at the sites of administration. No safety concerns were identified and formulations delivered therapeutically relevant concentrations to the skin with minimal systemic exposure.

After completion of Phase One of the study the 2% niclosamide Gel Formulation A was selected for further development.

### Example 3: A Double-Blind, Randomized, Intraindividual Vehicle-Controlled, Phase II Study to Evaluate Efficacy and Safety of Topically Applied Niclosamide in Human Patients with Moderate Atopic Dermatitis

The topical niclosamide Formulation G, as described in Table 2 above, was tested in the following clinical trial.

### Rationale for the Study

Wu et al (2014, Ibid) reports that niclosamide exhibited anti-inflammatory properties in vitro by modulating the activation of dendritic cells and repressing the expression of proinflammatory cytokines. This study will investigate whether niclosamide possesses anti-inflammatory properties capable of translating into a therapeutic effect on the signs and symptoms of atopic dermatitis.

### Study Design

31 patients with moderate atopic dermatitis (Investigator Global Assessment [IGA] of 3) were included in this double-blind, randomized, intraindividual vehicle-controlled, Phase 2 study to evaluate the efficacy and safety of topically applied niclosamide. Patients had at least 2 areas of at least 3×3-cm of atopic dermatitis with a Total Sign Score (TSS) of 5.2 patients discontinued the study before Day 22.

Patients received topical applications of a niclosamide 2% composition and vehicle once daily for 3 weeks, followed by a 1-week follow up period. Topical niclosamide 2% and vehicle was applied on two separate target lesions of atopic dermatitis (lesions of at least 3×3-cm that are at least 2 cm apart, excluding the face, scalp, genitals, hands, and feet). The application areas (5×5-cm) were randomized (1:1) to once daily application of niclosamide 2% or vehicle at 5 mg/cm² without occlusion 6 days per week. Patients came to the study site for all study product application for a total of 3 weeks.

Efficacy was assessed using Total Sign score (TSS) and Treatment Areas Assessment (TAA). Safety was assessed with vital signs, physical examination, clinical laboratory tests (haematology; biochemistry; urinalysis), and by collecting adverse events (AEs).

Three skin biopsies were collected in all patients (one from lesional skin at baseline, pre-dosing at Day 1, and two from lesional skin at Day 22 (one where topical niclosamide 2% had been applied and one where the vehicle had been applied). The lesional skin biopsies were analysed for skin thickness and inflammation biomarkers.

### Study Endpoints

### Primary Endpoint:

Number of local and systemic treatment-emergent adverse events (AEs) in each treatment group (during 34 days).

### Secondary Endpoints:

Change from baseline (pre-dosing at Day 1) in lesional TSS at Days 8, 15 and 22.
Change from baseline (pre-dosing at Day 1) in lesional Treatment Areas Assessment (TAA) at Days 8, 15 and 22, for area randomized to topically applied niclosamide 2%, as compared with vehicle.
Change from baseline in skin barrier and biomarker levels at Day 22, for area randomized to topically applied niclosamide 2%, as compared with vehicle.

### Inclusion Criteria

Patients will be eligible for participation in the study if they meet all the following inclusion criteria at the screening and baseline (pre-dosing at Day 1) visits, unless specified otherwise:
1. Man or woman 18 years of age or older at the time of consent.
2. Patient has clinically confirmed diagnosis of active atopic dermatitis, according to Hanifin and Rajka criteria (Hanifin et al. “Diagnostic feature of atopic dermatitis”, Acta. Derm. Ven. vol 92, (suppl):44-47, 1980).
3. Patient has at least a 6-month history of atopic dermatitis and had no significant flares in atopic dermatitis for at least 4 weeks before screening (information obtained from medical chart or patient's physician, or directly from the patient).
4. Patient has moderate atopic dermatitis at baseline (pre-dosing at Day 1), as defined by an IGA of 3.
5. Patient has at least two areas of atopic dermatitis (excluding face, scalp, genitals, hands, and feet) of at least 3×3 cm; with a TSS of at least 5 at baseline (Day 1). These areas should be at least 2 cm apart.
6. For patient (man and woman) involved in any sexual intercourse that could lead to pregnancy, patient agrees that an effective contraceptive method will be used, from at least 4 weeks before baseline (Day 1) until at least 4 weeks after the last study product administration. Effective contraceptive methods include hormonal contraceptives (combined oral contraceptive, patch, vaginal ring, injectable, or implant), intrauterine devices or intrauterine systems, vasectomy, tubal ligation, or a barrier method of contraception (male condom, female condom, cervical cap, diaphragm, contraceptive sponge) in conjunction with spermicide. Note: Hormonal contraceptives must have been on a stable dose for at least 4 weeks before baseline (Day 1).
   Note: Woman of nonchildbearing potential is as follows:
   Woman who has had surgical sterilization (hysterectomy, bilateral oophorectomy, or bilateral salpingectomy)
   Woman ≥40 years of age who has had a cessation of menses for at least 12 months and a follicle-stimulating hormone (FSH) test confirming nonchildbearing potential (refer to laboratory reference ranges for confirmatory levels) or cessation of menses for at least 24 months without FSH levels confirmed
7. For woman of childbearing potential, has had a negative serum pregnancy test at screening and negative urine pregnancy test at baseline (Day 1).
8. Patient is willing to participate and is capable of giving informed consent. Note: Consent must be obtained prior to any study-related procedures.

### Exclusion Criteria

Patients will not be eligible for participation in the study if they meet any of the following criteria at the screening and baseline (Day 1) visits, unless specified otherwise:
1. Patient is a woman who is breastfeeding, pregnant, or who is planning to become pregnant during the study.
2. Patient has clinically infected atopic dermatitis.
3. Patient has a Fitzpatrick's Skin Phototype >5.
4. Presence of any tattoos, scratches, open sores, excessive hair, or skin damages in the target lesion areas that in the opinion of the investigator may interfere with study evaluations.
5. Patient is known to have immune deficiency or is immunocompromised.
6. Patient has a history of cancer or lymphoproliferative disease within 5 years prior to baseline (Day 1). Patients with successfully treated nonmetastatic cutaneous squamous cell or basal cell carcinoma and/or localized carcinoma in situ of the cervix are not to be excluded.
7. Patient had a major surgery within 8 weeks prior to baseline (Day 1) or has a major surgery planned during the study.
8. Patient has any clinically significant medical condition or physical/laboratory/vital signs abnormality that would, in the opinion of the investigator, put the patient at undue risk or interfere with interpretation of study results.
9. Patient has a known history of chronic infectious disease (e.g., hepatitis B, hepatitis C, or infection with human immunodeficiency virus).
10. Patient has used hydroxyzine or diphenhydramine within 1 week prior to Day 1.
11. Patient has used dupilumab within 12 weeks prior to Day 1.
12. Patient has received any nonbiological investigational product or device within 4 weeks prior to Day 1
13. Patient has used crisaborole and any other topical PDE-4 inhibitor within 4 weeks prior to Day 1.
14. Patient has used doxepin within 1 week prior to Day 1.
15. Patient has used topical products containing urea on target areas within 1 week prior to baseline (Day 1).
16. Patient used nonurea-containing emollient anywhere on the body from 1 day before Day 1.
17. Patient has used systemic antibiotics within 2 weeks or topical antibiotics on target areas within 1 week prior to baseline (Day 1).
18. Patient has used any topical medicated treatment for atopic dermatitis within 1 week prior to baseline (Day 1), including, but not limited to, topical corticosteroids, calcineurin inhibitors, tars, bleach, antimicrobials, medical devices, and bleach baths.
19. Patient has used systemic treatments (other than biologics) that could affect atopic dermatitis less than 4 weeks prior to baseline (Day 1) (e.g., retinoids, calcineurin inhibitors, methotrexate, cyclosporine, hydroxycarbamide [hydroxyurea], azathioprine, oral/injectable corticosteroids). Note: Intranasal corticosteroids and inhaled corticosteroids for stable medical conditions are allowed if patient has been on a stable dose for at least 4 weeks prior to baseline (Day 1) and will continue usage at the same dose for the duration of the study. Eye drops containing corticosteroids are allowed.
20. Patient has received any marketed or investigational biological agent within 12 weeks or 5 half-lives (whichever is longer) prior to baseline (Day 1).
21. Patient has excessive sun exposure, is planning a trip to a sunny climate, or has used tanning booths within 4 weeks prior to baseline (Day 1), or is not willing to minimize natural and artificial sunlight exposure during the study. Use of sunscreen products and protective apparel are recommended when exposure cannot be avoided.
22. Patient has a known or suspected allergy to niclosamide or any component of the formulation to be tested.
23. Patient has a known history of clinically significant drug or alcohol abuse in the last year prior to baseline (Day 0).
24. Patient has a history of an allergic reaction or significant sensitivity to lidocaine or other local anaesthetics.
25. Patient has a history of hypertrophic scarring or keloid formation in scars or suture sites.
26. Patient is taking anticoagulant medication, such as heparin, low molecular weight (LMW)-heparin, warfarin, antiplatelets (nonsteroidal anti-inflammatory drugs [NSAIDs] and low-dose aspirin 581 mg will not be considered antiplatelets), or has a contraindication to skin biopsies.

### Diagnosis of AD

Diagnosis of AD in a subject will use the criteria according to Hanifin et al, ibid and set out in the Description of the present application. To be diagnosed with AD the subject should have at least three of the Major Criteria and at least three of the Minor Criteria

### Treatment

The study involved a comparison of the niclosamide topical composition with a matching vehicle, administered topically once daily for 3 weeks, without occlusion, at 5 mg/cm²/day (application areas 5×5 cm). The niclosamide formulation and placebo vehicle were applied on two separate target lesions of atopic dermatitis (lesions of at least 3×3 cm that are at least 2 cm apart, excluding the face, scalp, genitals, hands, and feet). As the chosen target lesion areas are expected to have a significant effect on outcomes, it is important to make a considerable effort to ensure select treatment areas with similar severity to reduce bias. Subjects came to the study site for all study product (active or vehicle) applications.

### Efficacy Assessments

Clinical evaluations of atopic dermatitis were performed by an experienced and qualified dermatologist (board certified or equivalent) or other suitably qualified and experienced designee. To assure consistency and reduce variability, the same assessor performed all assessments on a given subject whenever possible.

### Eczema Area and Severity Index

The Eczema Area and Severity Index (EASI) were assessed pre-dosing (Day 1). It quantifies the severity of the atopic dermatitis based on both lesion severity and the percentage of body surface area (BSA) affected. The EASI is a composite score ranging from 0 to 72 that takes into account the degree of erythema, induration/infiltration (papules), excoriation, and lichenification (each scored from 0 to 3 separately) for each of four body regions, with adjustment for the percentage of BSA involved for each body region and for the proportion of the body region to the whole body. The EASI score calculation is set out in the description.

### Body Surface Area

The overall BSA affected by atopic dermatitis was evaluated (from 0% to 100%)-pre-dosing (Day 1). For example, one subject's palm represents 1% of total BSA.

### Total Sign Score (TSS)

The lesional TSS on each of the two treatment areas was assessed pre-dosing (Day 1). It quantifies the severity of a subject's atopic dermatitis based on severity of erythema, edema/papulation, oozing/crusting, excoriation, lichenification, and dryness (each scored from 0 to 3, separately). The lesional TSS is a composite score ranging from 0 to 18. A detailed procedure of lesional TSS score calculation is set out in the description. To be eligible for this study, subjects had a TSS score of ≥5 pre-dosing (Day 1) for each treatment area.

### Treatment Areas Assessment (TAA)

The lesional TAA on each of the two treatment areas was assessed at the visits. The lesional TAA grades the severity of disease (each area scored from 0 to 5, separately).

### Skin Biopsies

Skin barrier and inflammation biomarker levels were determined from lesional skin biopsies from application areas. All subjects had a total of three skin biopsies: one biopsy at Day 1 and 2 biopsies at Day 22 (one where niclosamide was applied and one where the vehicle was applied).

Subjects who discontinued from the study but had completed at least the Day 15 visit, received treatment applications on Days 13 and 14, and received at least 12 applications up to Day 14, inclusively, had a biopsy taken as was planned for Day 22.

The skin biopsy samples were analysed by immunohistochemistry (IHC), and by gene expression studies by RT-PCR using TaqMan Low Density Array (TLDA), and by microarray using Affymetrix U133A Plus 2. The immunohistochemistry (IHC) was used to analyse cell biomarkers. The methodologies as disclosed by Guttman-Yassky et al, “Major differences in inflammatory dendritic cells and their products distinguish atopic dermatitis from psoriasis”, The Journal of Allergy and Clinical Immunology, vol. 119, issue 5, pages 1210-1217, 2007, were followed except for that U133A Plus 2-set Gene Chip probe arrays was used instead of U95A-set Gene Chip probe arrays.

### TLDA Data Analysis

Expression values (threshold cycle [Ct]) were normalized to Rplp0 by negatively transforming the Ct values to −dCt (IL17A was normalized to hARP, as analysed by qPCR). The undetected −dCt values were estimated for each gene as the 20% of the minimum across all samples. qRT-PCR expression data were modelled using a mixed effect model with Visit and Treatment Area as a fixed effect and a random intercept for each patient. This formulation intrinsically models the within patient correlation structure as in the case of a paired t-test. This approach introduces less bias than restricting the analysis for those patients who completed the study. Contrasts were used to estimate the fold changes with treatment within each treatment group and conduct hypothesis testing.

### Microarray Data Analysis

Experimental design: The hybridization strategy was in concordance with experimental design principles, by for example keeping all samples from the same patient in the same date, and always include samples from every treatment arm/group.

Quality Control and Pre-processing: Quality control of microarray chips were carried out using standard QC metrics and R package microarray Quality Control. Expression measures were obtained using GCRMA algorithm (Wu & Irizarry, 2004). Several visual and modelling techniques were used to elucidate if batch effect existed. Principal Component analysis plots were used to detect if any evident batch effect existed. If such batch effects were found, they were adjusted using Combat, an empirical Bayes method for adjusting data for batch effects that is robust to outliers in small sample sizes (Johnson, Li, & Rabinovic, 2007). The implementation of Combat by package sva was used.

Probe-sets with at least 5% samples with expression larger than 3 (in log 2-scale) were kept for further analysis. Expression values were modelled using mixed-effect models with fixed factors Visit and Treatment Area and a random effect for each patient. Fold changes for the comparisons of interest were estimated and hypothesis testing was conducted on such comparisons using contrasts under the general framework for linear models in limma package. The inter-replicate correlation was computed by Duplicate Correlation function and the linear model was estimated by ImFit. P-values from the moderated (paired) t-test were adjusted for multiple hypotheses using the Benjamini-Hochberg procedure, which controls for FDR.

### Statistical Analysis—TSS and TAA

Continuous variables were summarized in tables and included the number of patients, mean, standard deviation, median, minimum, and maximum. Categorical variables were presented in tables as frequencies and percentages.

The comparison between the treatment groups change from baseline in TSS at Day 22, was done using a paired Student t-test. The difference between treatments was estimated and presented along with a 95% confidence interval.

The other endpoints involving change from baseline were analysed using the same approach as described for the primary endpoint.

### Analysis Sets

Data from subjects who were randomized were included in the Intent to Treat (ITT) analysis set. Data from subjects who received at least one administration of study treatment on each lesion were included in the modified ITT (MITT) analysis set. Data were analyzed according to the treatment group to which the subject was randomized.

The Per Protocol (PP) analysis set included data from subjects who were randomized, had no significant protocol deviations effecting the efficacy assessment, and have evaluable data for the primary endpoint.

The Safety analysis set (SAF) was defined as data from subjects who received at least one administration of the study product. Analysis was performed according to the actual treatment subjects received.

### Efficacy Analysis—Lesional TSS at Day 22

The comparison between the treatment groups for change from baseline in lesional TSS at Day 22, was done using a paired Student t-test. The difference between treatments was estimated and presented along with a 95% confidence interval. Descriptive statistics for the baseline, Day 22 and change from baseline to Day 22 lesional TSS were presented for lesions treated with niclosamide and vehicle in the MITT population. Ninety-five percent confidence intervals (CIs) using a t-distribution were determined for the point estimates for change from baseline in each treatment group. Descriptive statistics and a 95% CI using a t-distribution will also be provided for the difference between the change from baseline in lesional TSS for the niclosamide and placebo lesions. Subjects with missing TSS at Day 22 were included in the analysis using a last observation carried forward imputation for the missing data. Analyses of the primary efficacy endpoint was repeated in the PP population.

Efficacy endpoints include TSS at Days 1 (pre-dosing), 8, 15 and 22, and TAA at Days 1 (pre-dosing), 8, 15 and 22. Analyses of endpoints were conducted in the same manner as described for the other efficacy endpoint.

### Efficacy Analysis—Biomarker/Clinical Score Correlation Analysis

The variables that were used for the correlation analysis were the clinical score (Total Sign Score (TSS) and Target Area Assessment (TAA)) of Day 22 and Baseline (Day 1) and the normalized biomarker expression values that were analysed with qRT-PCR (TLDA) and for the same days. The absolute change with treatment at Day 22 were calculated for each patient and each treatment. For the assessment of pairwise correlation the Spearman correlation coefficient was used. It is a non-parametric measure of rank correlation. The significant correlations were plotted with the respective linear regression line, a confidential interval of 95% and its respective rho (spearman coefficient, R) and p value. For this correlation analysis biomarkers were selected that showed significant changes in qRT-PCR and/or microarray. The correlation analysis was made on qRT-PCR data only except for the immune cells were IHC data was taken.

The same procedure was applied when analysing the correlation of individual scores and biomarker expression values. For this analysis biomarkers were taken that showed significant correlation to TSS or/and TAA. The correlation analysis was made on qRT-PCR data only.

### Biomarkers

Immune effectors (herein also referred to as biomarkers) included in the immunohistochemistry (IHC) and in the gene expression analysis using qRT-PCR were grouped as shown in Table 7:

TABLE 7

| Group | Biomarkers |
|---|---|
| General inflammation | MMP12 |
| Proliferation | KRT16 |
| Th-1 related | CXCL10, IFNg, IL12A, |
| | CXCL9 |
| Th2-related | IL13, IL10, IL33, TSLPR, |
| | IL31, IL5, IL9 |
| | CCL17, CCL22, CCL18. |
| | CCL26 |
| Innate immunity | IL6, IL8, IL17C, IL1B |
| Skin barrier/Terminal | LOR, FLG, PPL |
| differentiation | |
| Th17/Th22-related | S100A8, S100A12, S100A7, |
| | S100A9, IL22 |
| Th17-related | IL17A, IL17F, IL23A, CAMP, |
| | IL19, IL12B |
| | DEFB4A/DEFB4B, CXCL1, |
| | CXCL2, CCL20, PI3 |
| T-Cell/NK cell activation | IL15, IL15RA, IL2, CCL5 |
| T regulatory cell | FOXP3 |
| Dendritic cells | CD11c, FceR1 |
| Langerhans Cells | Langerin |
| T cells | CD3 |

Thymic stromal lymphopoietin protein receptor (TSLP-R) is the receptor for the proinflammatory cytokine thymic stromal lymphopoietin (TSLP).

CD3 (cluster of differentiation 3) is a biomarker for T cells.

FOXP3 (also known as scurfin) is a biomarker for a subpopulation of T cells called regulatory T cells (also known as suppressor T cells).

As mentioned above, the biomarkers that showed significant changes in qRT-PCR (TLDA) expression analysis were selected for correlation analysis with TSS and TAA.

Further biomarkers were included in the microarray analysis, see Tables 14-17.

### Results

### Skin Thickness at Day 22

No differences in skin thickness were found following treatment with 2% niclosamide compared to baseline and compared to vehicle.

### Expression Levels of Biomarkers at Day 22 and Correlation Versus Total Severity Score (TSS) and Target Area Assessment (TAA)

Biomarkers were analysed by qRT-PCR or microarray in the skin biopsies taken at Day 1 and at Day 22 as described hereinbefore.

The results for all biomarkers analysed by qRT-PCR are presented in Tables 8-13.

The results for all biomarkers analysed by microarray are presented in Tables 14-16.

TABLE 8
qRT-PCR - all Biomarkers results

| | | Vehicle | | Niclosamide | | Niclosamide | |
|---|---|---|---|---|---|---|---|
| | Biomarker | vs. Predose | | vs. Predose | | vs. Vehicle | |
| General Inflammation | MMP12 | −1.72 | + | −3.27 | *** | −1.90 | + |
| Innate Immunity Markers | IL1B | −1.63 | * | −1.64 | * | −1.01 | |
| Innate Immunity Markers | IL8 | −2.59 | * | −3.78 | ** | −1.46 | |
| Innate Immunity Markers | IL6 | −1.89 | * | −2.34 | ** | −1.24 | |
| Innate Immunity Markers | IL17C | −1.38 | | −3.24 | *** | −2.35 | ** |
| Skin barrier/Proliferation | KRT16 | −2.43 | ** | −3.88 | *** | −1.60 | |
| T cell Activation Marker | IL2 | 1.13 | | −1.20 | | −1.36 | |
| T cell Activation Marker | CCL5 | −1.19 | | −1.22 | | −1.03 | |
| T cell Activation Marker | IL15 | −1.29 | * | −1.47 | *** | −1.14 | |
| T cell Activation Marker | IL15RA | −1.31 | ** | −1.46 | *** | −1.12 | |
| T regulatory cell Marker | FOXP3 | −1.22 | | −1.39 | * | −1.14 | |
| Skin barrier/Terminal | PPL | −1.11 | | −1.07 | | 1.04 | |
| Differentiation Marker | | | | | | | |
| Skin barrier/Terminal | FLG | 1.16 | | 1.50 | * | 1.30 | |
| Differentiation Marker | | | | | | | |
| Skin barrier/Terminal | LOR | 1.32 | | 1.67 | * | 1.27 | |
| Differentiation Marker | | | | | | | |
| TH1 related genes | IFNg | −1.32 | | −1.11 | | 1.19 | |
| TH1 related genes | IL12A | −1.09 | | −1.01 | | 1.09 | |
| TH1 related genes | CXCL9 | −1.75 | * | −1.51 | + | 1.16 | |
| TH1 related genes | CXCL10 | −1.49 | | −1.64 | * | −1.10 | |
| Th17 chemokine | CCL20 | 1.15 | | −1.96 | ** | −2.25 | *** |
| related genes | | | | | | | |
| Th17 chemokine | CXCL2 | −1.56 | * | −1.89 | ** | −1.21 | |
| related genes | | | | | | | |
| Th17 chemokine | DEFB4A/ | −1.98 | + | −4.09 | *** | −2.06 | + |
| related genes | DEFB4B | | | | | | |
| Th17 chemokine | CXCL1 | −1.72 | + | −2.80 | *** | −1.63 | + |
| related genes | | | | | | | |
| Th17 chemokine | PI3 | −2.34 | * | −4.52 | *** | −1.93 | + |
| related genes | | | | | | | |
| Th17 cytokine | IL17F | −1.34 | | 1.02 | | 1.37 | |
| related genes | | | | | | | |
| Th17 cytokine | IL19 | −3.77 | * | −3.26 | * | 1.16 | |
| related genes | | | | | | | |
| Th17 cytokine | CAMP | −2.27 | * | −3.02 | *** | −1.33 | |
| related genes | | | | | | | |
| Th17 cytokine | IL12B | −1.42 | | −2.01 | * | −1.42 | |
| related genes | | | | | | | |
| Th17 cytokine | IL23A | −1.56 | * | −2.13 | *** | −1.37 | |
| related genes | | | | | | | |
| Th17 cytokine | IL17A | −1.60 | + | −2.23 | ** | −1.40 | |
| related genes | | | | | | | |
| Th17/TH22 related genes | S100A7 | −1.38 | | −3.04 | *** | −2.20 | ** |
| Th17/TH22 related genes | IL22 | −1.45 | | −2.86 | ** | −1.97 | + |
| Th17/TH22 related genes | S100A9 | −1.76 | + | −3.47 | *** | −1.97 | * |
| Th17/TH22 related genes | S100A8 | −1.91 | + | −3.85 | *** | −2.02 | + |
| Th17/TH22 related genes | S100A12 | −2.03 | + | −4.29 | *** | −2.11 | + |
| Th2 related chemokines | CCL26 | 1.44 | * | −1.03 | | −1.49 | * |
| Th2 related chemokines | CCL18 | −1.21 | | −2.45 | *** | −2.02 | ** |
| Th2 related chemokines | CCL22 | −1.32 | | −1.95 | ** | −1.49 | + |
| Th2 related chemokines | CCL17 | −1.48 | | −2.02 | * | −1.37 | |
| Th2 related genes | IL33 | −1.21 | + | −1.08 | | 1.11 | |
| Th2 related genes | IL31 | −1.27 | | −1.85 | | −1.46 | |
| Th2 related genes | IL10 | −1.23 | | −1.85 | *** | −1.51 | ** |
| Th2 related genes | IL13 | −1.29 | | −2.06 | * | −1.59 | |
| Th2 related genes | IL5 | −1.62 | | −2.38 | * | −1.47 | |
| Th9 | IL9 | −1.62 | | −1.91 | | −1.17 | |
| Th2 related genes | TSLPR | −1.33 | | −1.82 | ** | −1.36 | |

*** (p < 0.001)
** (p < 0.01)
* (p < 0.05) + (p < 0.1)

TABLE 9
Biomarkers that changed significant with treatment
at Day 22 compared to Baseline (qRT-PCR)

| | | Niclosamide | |
|---|---|---|---|
| | Biomarker | vs Predose | |
| General Inflammation | Matrix Metallopeptidase | −3.27 | *** |
| | 12 (MMP12) | | |
| Innate Immunity Markers | Interleukin 1B (IL1B) | −1.64 | * |
| Innate Immunity Markers | Interleukin 8 (IL8) | −3.78 | ** |
| Innate Immunity Markers | Interleukin 6 (IL6) | −2.34 | ** |
| Innate Immunity Markers | Interleukin 17C (IL17C) | −3.24 | *** |
| Skin barrier/Proliferation | Keratin 16 (KRT16) | −3.88 | *** |
| T cell Activation Marker | Interleukin 15 (IL15) | −1.47 | *** |
| T cell Activation Marker | Interleukin 15RA (IL15RA) | −1.46 | *** |
| T regulatory Marker | Forkhead Box P3 (FOXP3) | −1.39 | * |
| Skin barrier/Terminal | Filaggrin (FLG) | 1.50 | * |
| Differentiation Marker | | | |
| Skin barrier/Terminal | Loricrin (LOR) | 1.67 | * |
| Differentiation Marker | | | |
| TH1 related genes | Chemokine (C-X-C Motif) | −1.64 | * |
| | Ligand 10 (CXCL10) | | |
| Th17 chemokine related genes | Chemokine (C-C Motif) | −1.96 | ** |
| | Ligand 20 (CCL20) | | |
| Th17 chemokine related genes | Chemokine (C-X-C Motif) | −1.89 | ** |
| | Ligand 2 (CXCL2) | | |
| Th17 chemokine related genes | Defensin Beta 4A/B | −4.09 | *** |
| | (DEFB4A/DEFB4B) | | |
| Th17 chemokine related genes | Chemokine (C-X-C Motif) | −2.80 | *** |
| | Ligand 1 (CXCL1) | | |
| Th17 chemokine related genes | Peptidase Inhibitor 3 (PI3) | −4.52 | *** |
| Th17 cytokine related genes | Interleukin 19 (IL19) | −3.26 | * |
| Th17 cytokine related genes | Cathelicidin Antimicrobial | −3.02 | *** |
| | Peptide (CAMP) | | |
| Th17 cytokine related genes | Interleukin 12B (IL12B) | −2.01 | * |
| Th17 cytokine related genes | Interleukin 23A (IL23A) | −2.13 | *** |
| Th17 cytokine related genes | Interleukin 17A (IL17A) | −2.23 | ** |
| Th17/TH22 related genes | S100 Calcium Binding | −3.04 | *** |
| | Protein 7 (S100A7) | | |
| Th17/TH22 related genes | Interleukin 22 (IL22) | −2.86 | ** |
| Th17/TH22 related genes | S100 Calcium Binding | −3.47 | *** |
| | Protein 9 (S100A9) | | |
| Th17/TH22 related genes | S100 Calcium Binding | −3.85 | *** |
| | Protein 8 (S100A8) | | |
| Th17/TH22 related genes | S100 Calcium Binding | −4.29 | *** |
| | Protein 12 (S100A12) | | |
| Th2 related chemokines | Chemokine (C-C Motif) | −2.45 | *** |
| | Ligand 18 (CCL18) | | |
| Th2 related chemokines | Chemokine (C-C Motif) | −1.95 | ** |
| | Ligand 22 (CCL22) | | |
| Th2 related chemokines | Chemokine (C-C Motif) | −2.02 | * |
| | Ligand 17 (CCL17) | | |
| Th2 related genes | Interleukin 10 (IL10) | −1.85 | *** |
| Th2 related genes | Interleukin 13 (IL13) | −2.06 | * |
| Th2 related genes | Interleukin 5 (IL5) | −2.38 | * |
| Th2 related genes | TSLPR | −1.82 | ** |

*** (p < 0.001)
** (p < 0.01)
* (p < 0.05) + (p < 0.1)

TABLE 10
Biomarkers that are significant changed with
treatment compared to vehicle (qRT-PCR)

| | | Niclosamide | |
|---|---|---|---|
| | Biomarker | vs vehicle | |
| Innate Immunity Markers | Interleukin 17C (IL17C) | −2.35 | ** |
| Th17 chemokine | Chemokine (C-C Motif) | −2.25 | *** |
| related genes | Ligand 20 (CCL20) | | |
| Th17/TH22 | S100 Calcium Binding | −2.20 | ** |
| related genes | Protein 7 (S100A7) | | |
| Th17/TH22 | S100 Calcium Binding | −1.97 | * |
| related genes | Protein 9 (S100A9) | | |
| Th2 related | Chemokine (C-C Motif) | −1.49 | * |
| chemokines | Ligand 26 (CCL26) | | |
| Th2 | Chemokine (C-C Motif) | −2.02 | ** |
| related chemokines | Ligand 18 (CCL18) | | |
| Th2 related | Interleukin 10 (IL10) | −1.51 | ** |
| genes | | | |

*** (p < 0.001)
** (p < 0.01)
* (p < 0.05) + (p < 0.1)

TABLE 11
Biomarkers that are significant changed to Baseline
and vehicle with Niclosamide (qRT-PCR)

| | | Niclosamide | | Niclosamide | |
|---|---|---|---|---|---|
| | | vs. Predose | | vs. vehicle | |
| Innate Immunity | Interleukin 17C | −3.24 | *** | −2.35 | ** |
| Markers | (IL17C) | | | | |
| Th17 chemokine | Chemokine (C-C | −1.96 | ** | −2.25 | *** |
| related genes | Motif) Ligand | | | | |
| | 20 (CCL20) | | | | |
| Th17/TH22 | S100 Calcium | −3.04 | *** | −2.20 | ** |
| related genes | Binding Protein | | | | |
| | 7 (S100A7) | | | | |
| Th17/TH22 | S100 Calcium | −3.47 | *** | −1.97 | * |
| related genes | Binding Protein | | | | |
| | 9 (S100A9) | | | | |
| Th2 related | Chemokine (C-C | −2.45 | *** | −2.02 | ** |
| chemokines | Motif) Ligand | | | | |
| | 18 (CCL18) | | | | |
| Th2 related genes | Interleukin | −1.85 | *** | −1.51 | ** |
| | 10 (IL10) | | | | |

*** (p < 0.001)
** (p < 0.01)
* (p < 0.05) + (p < 0.1)

TABLE 12
Significant correlations of biomarker expression
(based on qRT-PCR/IHC data) to TSS at Day 22

| | | TSS_p |
|---|---|---|
| | TSS | value |
| S100A8 | 0.829 | 0.000 |
| S100A7 | 0.794 | 0.000 |
| KRT16 | 0.769 | 0.000 |
| S100A9 | 0.761 | 0.000 |
| S100A12 | 0.694 | 0.000 |
| PI3 | 0.689 | 0.000 |
| IL13 | 0.681 | 0.000 |
| IL22 | 0.670 | 0.000 |
| DEFB4A/DEFB4B | 0.601 | 0.001 |
| CCL17 | 0.585 | 0.001 |
| MMP12 | 0.545 | 0.002 |
| LOR | −0.516 | 0.004 |
| CCL22 | 0.502 | 0.006 |
| IL17A | 0.479 | 0.009 |
| IL19 | 0.472 | 0.010 |
| CD11c_Dermis | 0.460 | 0.012 |
| IL8 | 0.420 | 0.023 |
| FLG | −0.406 | 0.029 |
| CXCL1 | 0.377 | 0.044 |
| CAMP | 0.374 | 0.046 |

TABLE 13
Significant correlation of biomarker expression
(based on qRT-PCR data) to TAA at Day 22

| | | TAA_p |
|---|---|---|
| | TAA | value |
| KRT16 | 0.694 | 0.000 |
| S100A7 | 0.667 | 0.000 |
| S100A8 | 0.658 | 0.000 |
| S100A9 | 0.643 | 0.000 |
| IL13 | 0.641 | 0.000 |
| IL22 | 0.632 | 0.000 |
| CCL17 | 0.599 | 0.001 |
| MMP12 | 0.590 | 0.001 |
| S100A12 | 0.553 | 0.002 |
| PI3 | 0.526 | 0.003 |
| DEFB4A/DEFB4B | 0.518 | 0.004 |
| IL19 | 0.456 | 0.013 |
| IL8 | 0.500 | 0.006 |
| CCL22 | 0.440 | 0.017 |
| LOR | −0.432 | 0.019 |
| FLG | −0.408 | 0.028 |

TABLE 14
Biomarker expression levels that changed significant
with treatment compared to Baseline (Microarray)

| | | Niclosamide | |
|---|---|---|---|
| | Biomarker | vs Predose | |
| Dendritic cells | CD86 | −1.60 | *** |
| General Inflammation | CCL19 | −1.56 | ** |
| General Inflammation | IL24 | −2.19 | ** |
| General Inflammation | MMP12 | −2.81 | ** |
| skin barrier lipids | ACOX2 | 1.40 | * |
| skin barrier lipids | ACSL1 | 1.21 | *** |
| unsorted | ANXA6 | −1.24 | * |
| skin barrier lipids | CDSN | 1.14 | ** |
| skin barrier lipids | CERS3 | 1.13 | * |
| skin barrier lipids | CLN8 | 1.15 | * |
| skin barrier lipids | ELOVL3 | 1.81 | * |
| skin barrier lipids | EREG | 1.19 | * |
| skin barrier lipids | FA2H | 1.50 | * |
| skin barrier lipids | FAR2 | 1.48 | * |
| skin barrier lipids | KRT79 | 1.91 | * |
| skin barrier lipids | PNPLA3 | 1.91 | ** |
| skin barrier lipids | PPL | 1.06 | * |
| skin barrier, epidermal differentiation | ACER1 | 1.94 | *** |
| skin barrier, epidermal differentiation | ANXA9 | 1.56 | *** |
| skin barrier, epidermal differentiation | CLDN1 | 1.17 | * |
| skin barrier, epidermal differentiation | CLDN23 | 1.42 | ** |
| skin barrier, epidermal differentiation | DGAT2 | 1.33 | *** |
| skin barrier, epidermal differentiation | DHCR7 | 1.12 | * |
| skin barrier, epidermal differentiation | FAXDC2 | 1.46 | ** |
| skin barrier, epidermal differentiation | FLG | 1.17 | * |
| skin barrier, epidermal differentiation | KRT23 | 1.26 | *** |
| skin barrier, epidermal differentiation | KRT77 | 1.43 | ** |
| skin barrier, epidermal differentiation | SCEL | 1.43 | *** |
| unsorted | SPTLC3 | 1.44 | *** |
| skin barrier, epidermal differentiation | TJP3 | 1.51 | ** |
| T cell activation | CCR7 | −1.51 | ** |
| T cell activation | CD2 | −1.56 | * |
| T cell activation | CD28 | −1.72 | * |
| T cell activation | CD3D | −1.63 | ** |
| T cell activation | CD3G | −1.62 | * |
| Th1 | CCL2 | −1.77 | ** |
| Th1 | CCR1 | −1.90 | *** |
| Th1 | CCR2 | −1.37 | * |
| Th1 | IFNGR2 | −1.13 | ** |
| Th1 | IL12RB2 | −1.21 | * |
| Th1 | IL2RA | −1.27 | * |
| Th1 | IRF1 | −1.37 | * |
| Th17 | CCR6 | −1.33 | * |
| Th17 | CXCL1 | −2.83 | ** |
| Th17 | CXCL2 | −1.81 | * |
| Th17 | IL6R | −1.25 | *** |
| Th17 | LCN2 | −1.62 | * |
| Th17 | PI 3 | −3.13 | *** |
| Th17 | STAT3 | −1.13 | * |
| Th17 | IL37 | 1.69 | * |
| TH17 | TNFSF4 | −1.64 | *** |
| Th17/Th22 | S100A12 | −3.62 | *** |
| Th17/Th22 | S100A7 | −1.17 | * |
| Th17/Th22 | S100A8 | −1.45 | ** |
| Th17/Th22 | S100A9 | −2.81 | *** |
| Th17/Th22 | S100P | −1.35 | * |
| Th17/Th22 | SERPINB1 | −1.29 | *** |
| Th17/Th22 | SERPINB4 | −1.54 | * |
| Th2 | CCL13 | −1.40 | ** |
| Th2 | CCL18 | −1.70 | ** |
| Th2 | CCL22 | −1.83 | ** |
| Th2 | CCR5 | −1.40 | ** |
| Th2 | IL4R | −1.80 | *** |
| Th2 | IL7R | −1.58 | * |
| unsorted | IL1F10 | 1.98 | *** |

TABLE 15
Biomarker expression (microarray) that changed
significant with treatment compared to vehicle

| | | Vehicle | |
|---|---|---|---|
| | Biomarker | vs. Niclosamide | |
| unsorted | CCL23 | 1.63 | * |
| unsorted | IL26 | 1.78 | * |
| unsorted | ACOT2 | −1.35 | * |
| skin barrier lipids | ACOX2 | −1.40 | * |
| skin barrier lipids | ELOVL3 | −1.66 | ** |
| skin barrier lipids | FA2H | −1.57 | * |
| skin barrier lipids | FAR2 | −1.47 | * |
| skin barrier lipids | KRT79 | −2.00 | * |
| skin barrier lipids | PNPLA3 | −1.79 | ** |
| unsorted | PPARG | −1.55 | * |
| skin barrier, epidermal differentiation | DGAT2 | −1.19 | * |
| skin barrier, epidermal differentiation | FAXDC2 | −1.29 | * |
| unsorted | SPTLC3 | −1.30 | ** |
| Th1 | CCL2 | 1.56 | * |
| Th1 | CCR1 | 1.55 | ** |
| Th1 | IFNGR2 | 1.10 | * |
| Th1 | STAT1 | 1.13 | * |
| Th17 | CCL20 | 2.00 | * |
| Th17 | CCR6 | 1.31 | * |
| Th17 | CXCL1 | 2.10 | * |
| Th17 | LCN2 | 1.69 | * |
| Th17 | PI 3 | 1.87 | * |
| Th17 | STAT3 | 1.16 | ** |
| TH17 | TNFSF4 | 1.35 | * |
| Th17/Th22 | S100A12 | 2.30 | * |
| Th17/Th22 | S100A9 | 1.88 | * |
| Th2 | CCL13 | 1.32 | * |
| Th2 | CCL18 | 1.66 | * |
| Th2 | CCL26 | 1.69 | * |
| Th2 | IL4R | 1.29 | * |
| unsorted | IL1F10 | −1.79 | *** |

TABLE 16
Biomarkers that are significant changed compared to
Baseline and vehicle with treatment (Microarray)

| | | Niclosamide | | Vehicle | |
|---|---|---|---|---|---|
| | Biomarker | vs Predose | | vs. Niclosamide | |
| skin barrier lipids | ACOX2 | 1.40 | * | −1.40 | * |
| skin barrier lipids | ELOVL3 | 1.81 | * | −1.66 | * |
| skin barrier lipids | FA2H | 1.50 | * | −1.57 | * |
| skin barrier lipids | FAR2 | 1.48 | * | −1.47 | * |
| skin barrier lipids | KRT79 | 1.91 | * | −2.00 | * |
| skin barrier lipids | PNPLA3 | 1.91 | ** | −1.79 | ** |
| skin barrier, epidermal | DGAT2 | 1.33 | *** | −1.19 | * |
| differentiation | | | | | |
| skin barrier, epidermal | FAXDC2 | 1.46 | ** | −1.29 | * |
| differentiation | | | | | |
| unsorted | SPTLC3 | 1.44 | *** | −1.30 | ** |
| Th1 | CCL2 | −1.77 | ** | 1.56 | * |
| Th1 | CCR1 | −1.90 | *** | 1.55 | ** |
| Th1 | IFNGR2 | −1.13 | ** | 1.10 | * |
| Th17 | CCR6 | −1.33 | * | 1.31 | * |
| Th17 | CXCL1 | −2.83 | ** | 2.10 | * |
| Th17 | LCN2 | −1.62 | * | 1.69 | * |
| Th17 | PI 3 | −3.13 | *** | 1.87 | * |
| Th17 | STAT3 | −1.13 | * | 1.16 | ** |
| TH17 | TNFSF4 | −1.64 | *** | 1.35 | * |
| Th17/Th22 | S100A12 | −3.62 | *** | 2.30 | * |
| Th17/Th22 | S100A9 | −2.81 | *** | 1.88 | * |
| Th2 | CCL13 | −1.40 | ** | 1.32 | * |
| Th2 | CCL18 | −1.70 | ** | 1.66 | * |
| Th2 | IL4R | −1.80 | *** | 1.29 | * |
| unsorted | IL1F10 | 1.98 | *** | −1.79 | *** |

### Conclusions

Significant changes from baseline (pre-dosing at Day 1) were found for certain immune effectors in the biopsies taken at Day 22.

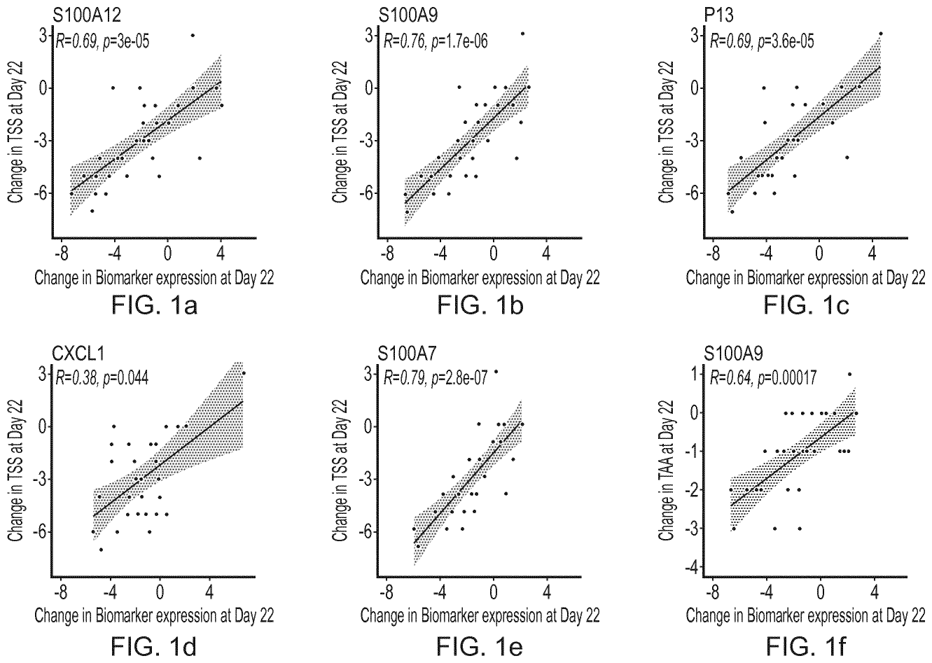

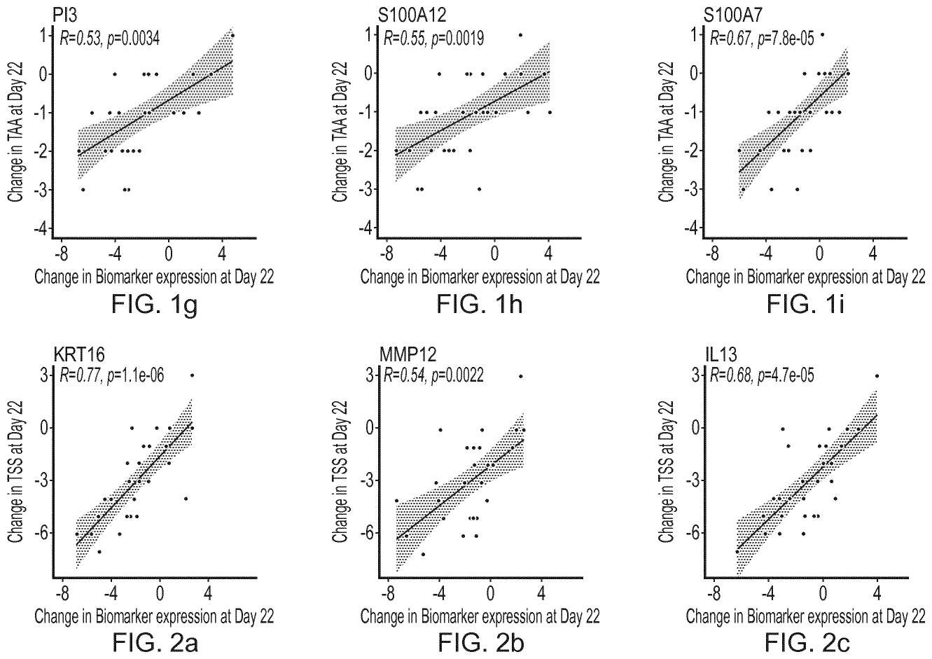

S100A12 was found to be significantly downregulated at Day 22 following topical administration of 2% niclosamide compared to baseline (−3.62) and compared to vehicle (−2.30), p<0.05). S100A12 was found to be significantly correlated with TSS and TAA. Results are shown in FIGS. 1a and 1h, respectively. The graphs show the correlation of change in biomarker expression at Day 22 compared to baseline to change in TSS at Day 22.

S100A9 was found to be significantly downregulated at Day 22 following topical administration of 2% niclosamide compared to baseline (−2.81) and compared to vehicle (−1.88) (p<0.05). S100A9 was found to be significantly correlated with TSS and TAA. Results are shown in FIGS. 1b and 1f respectively. The graphs show the correlation change in biomarker expression at Day 22 compared to baseline to change in TSS at Day 22.

PI3 was found to be significantly downregulated at Day 22 following topical administration of 2% niclosamide compared to baseline (−3.13) and compared to vehicle (−1.87) (p<0.05). PI3 was found to be significantly correlated to TSS and TAA. Results are shown in FIGS. 1c and 1g respectively. The graphs show the correlation of change in biomarker expression at Day 22 compared to baseline to change in TSS at Day 22.

CXCL1 was found to be significantly downregulated at Day 22 following topical administration of 2% niclosamide compared to baseline (−2.83) and compared to vehicle (−2.10) (p<0.05). CXCL1 was found to be significantly correlated to TSS. Results are shown in FIG. 1d. The graphs show the correlation of change in biomarker expression at Day 22 compared to baseline to change in TSS at Day 22.

S100A7 was found to be significantly downregulated at Day 22 following topical administration of 2% niclosamide compared to baseline (−3.04) and compared to vehicle (−2.20) (p<0.05). S100A7 was found to be significantly correlated to TSS and TAA. Results are shown in FIGS. 1e and 1i, respectively. The graphs show the correlation of change in biomarker expression at Day 22 compared to baseline to change in TSS at Day 22.

Thus, S100A12, S100A9, PI3, S100A7 and CXCL1 were all shown to be significantly downregulated in expression compared to baseline as well as vehicle and were all found to be clinically correlated to TSS.

Among these biomarkers that showed significant change compared to vehicle and baseline, S100A7 and S100A9 were found to have the highest correlations to TSS and S100A7 and S100A9 to were found to have the highest correlations to TAA.

The levels of the biomarkers listed in Table 9 above and analysed by qRT-PCR were also found to have changed significantly at Day 22 compared to baseline following topical administration of 2% niclosamide.

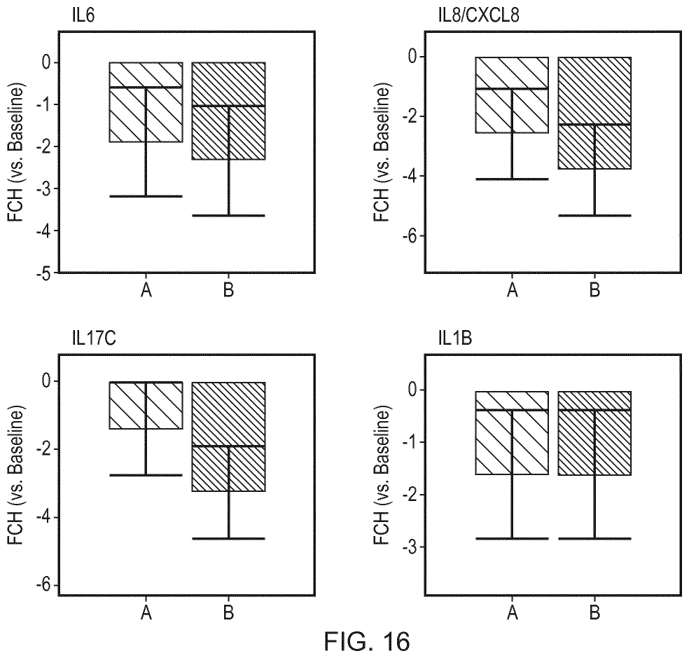

Results are shown in FIGS. 16-25, where A denotes vehicle and B denotes niclosamide at Day 22 compared to baseline.

FIG. 16 shows changes in biomarkers (IL6, IL8, IL170, IL1B) associated with innate immunity.

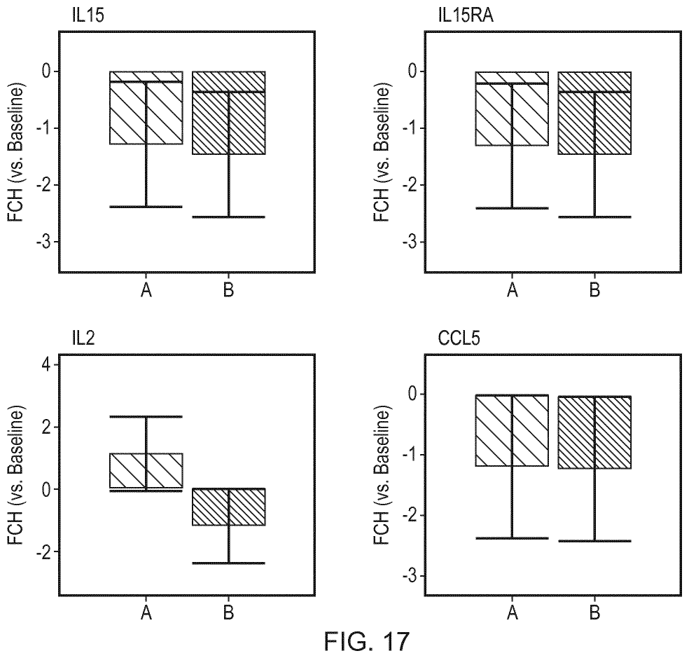

FIG. 17 shows changes in biomarkers (IL15, IL15RA, IL2, CCLS) associated with T cell activation.

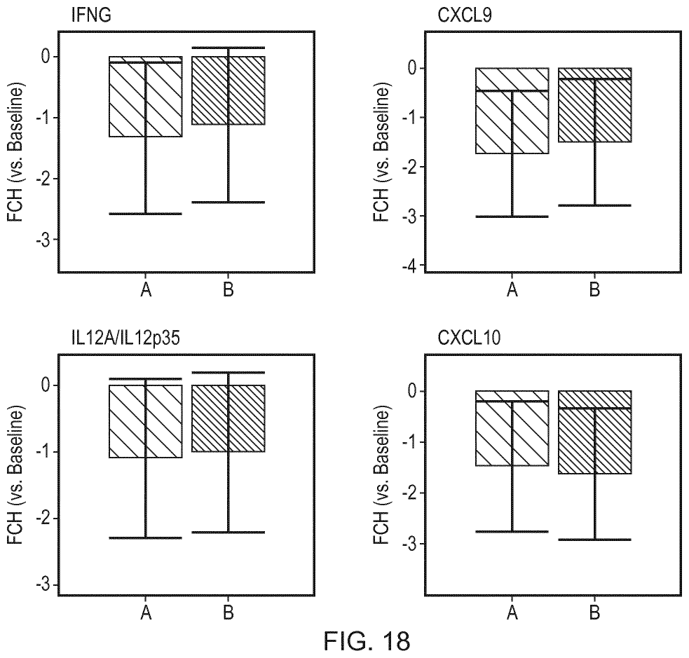

FIG. 18 shows changes in biomarkers (IFNG, CXCL9, IL12A/IL12p35, CXCL10) associated with Th1 related genes.

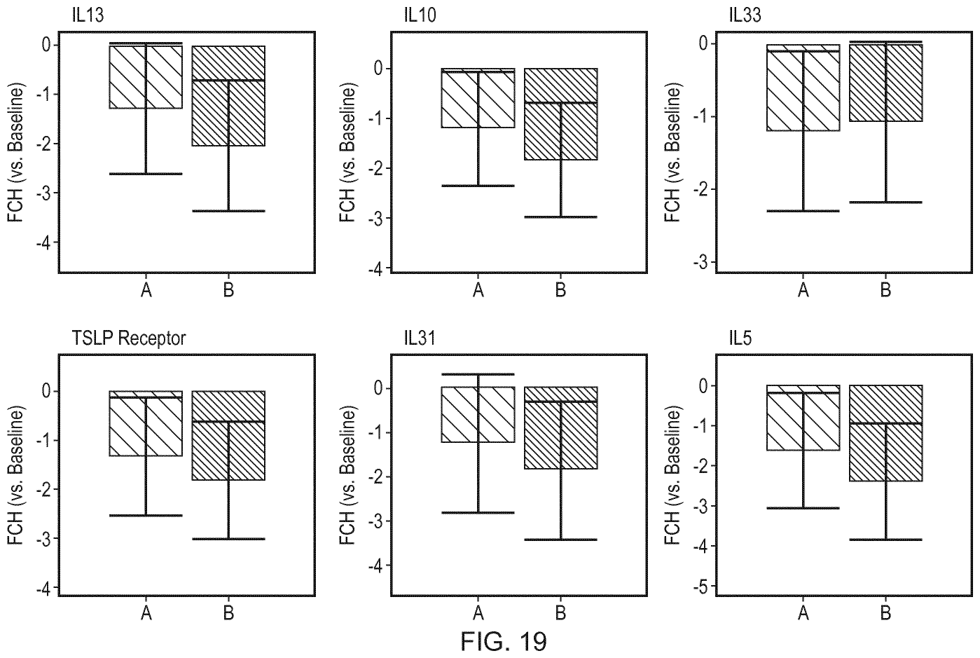

FIG. 19 shows changes in biomarkers (IL13, 11_10, IL33, TSLP-R, IL31, IL5) associated with Th2 related genes.

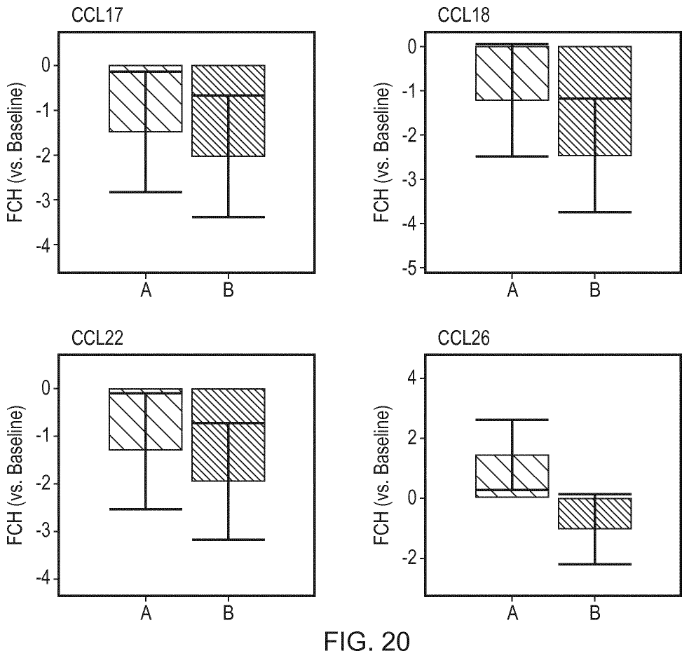

FIG. 20 shows changes in biomarkers (CCL17, CCL18, CCL22, CCL26) associated with Th2 related chemokines.

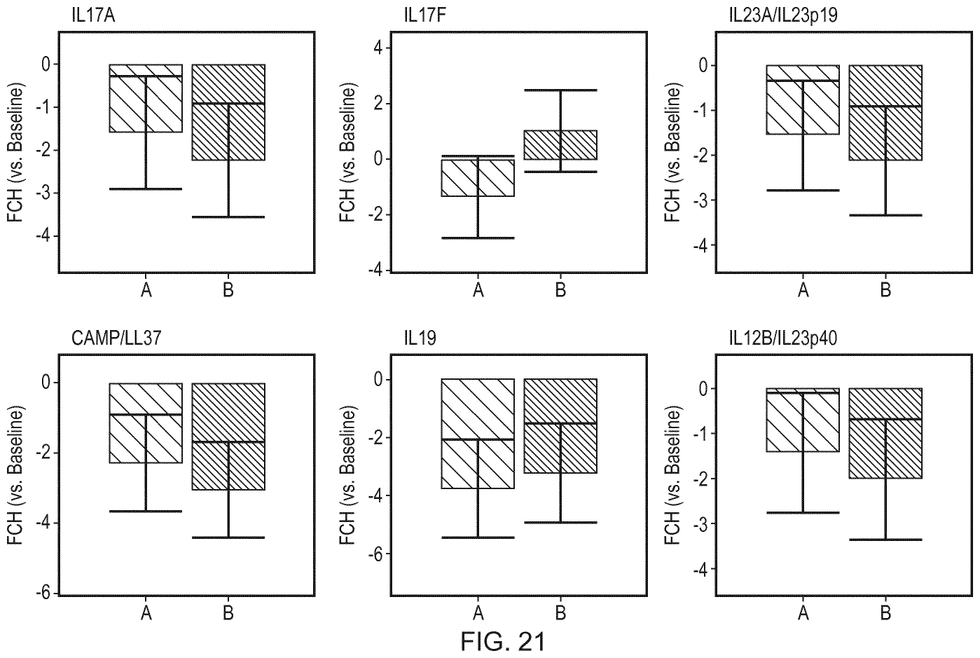

FIG. 21 shows changes in biomarkers (IL17A, IL17F, IL23A/IL23p19, CAMP/LL37, IL19, IL12B/IL23p40) associated with Th17 cytokine related genes.

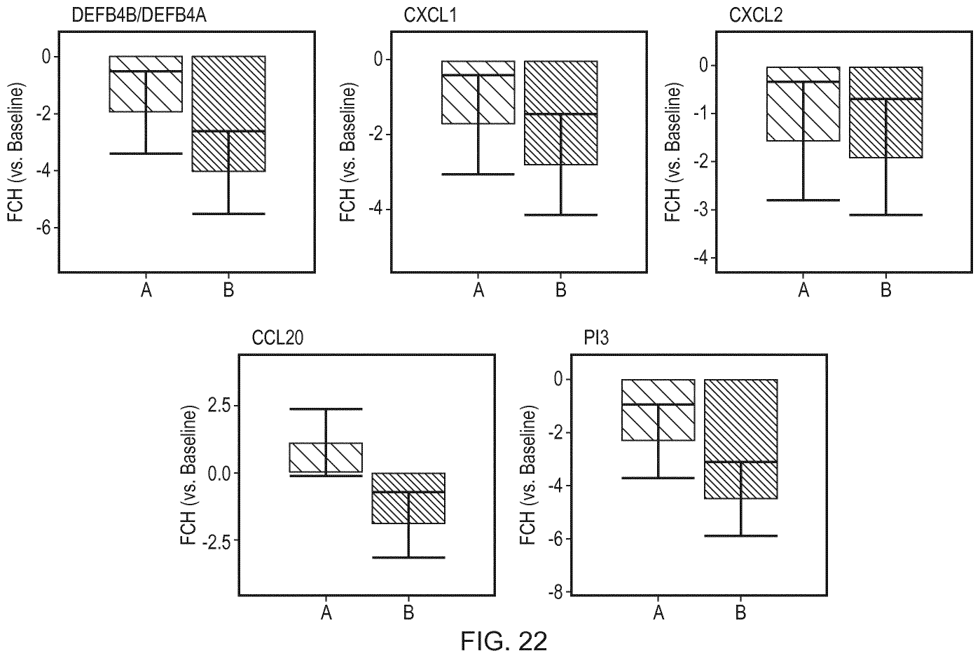

FIG. 22 shows changes in biomarkers (DEFB4A/DEFB4B, CXCL1, CXCL2, CCL20, PI3) associated with Th17 chemokine related genes.

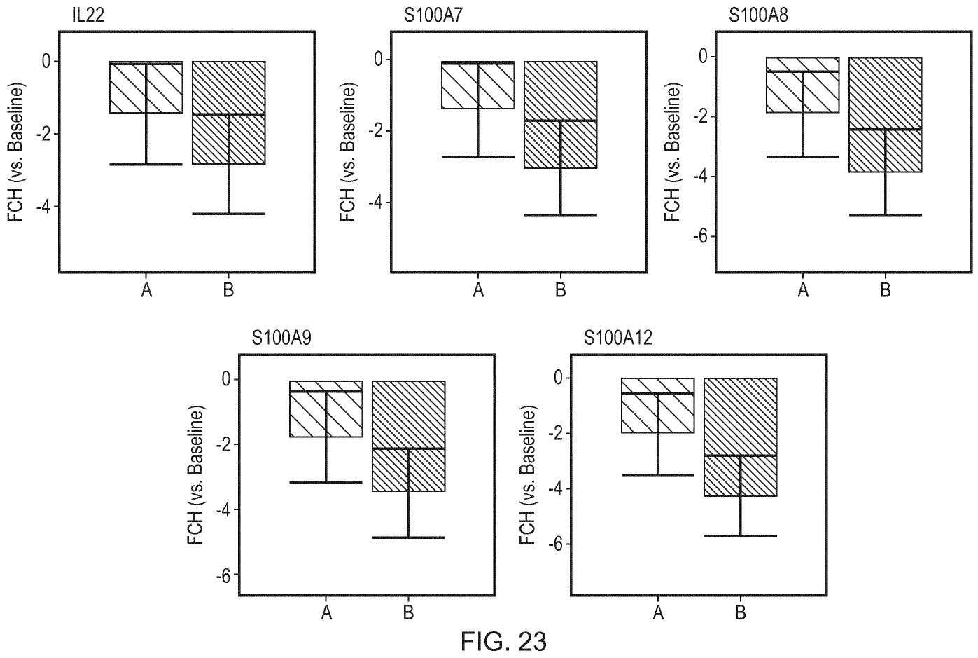

FIG. 23 shows changes in biomarkers (IL22, S100A7, S100A8, S100A9, S100A12) associated with Th17/Th22 related genes.

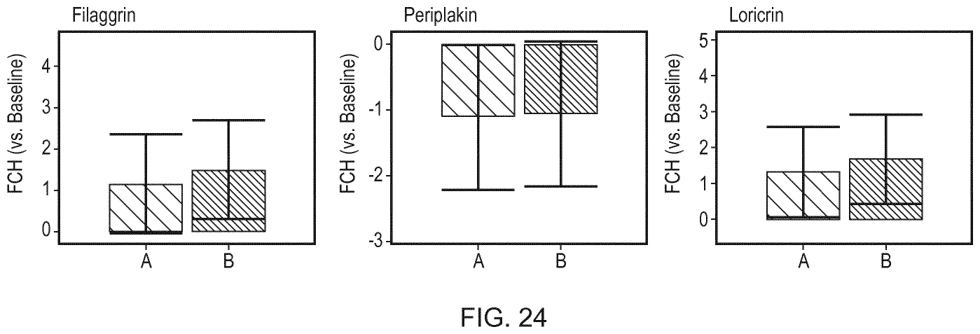

FIG. 24 shows changes in biomarkers (FLG, PPL, LOR) associated with terminal differentiation.

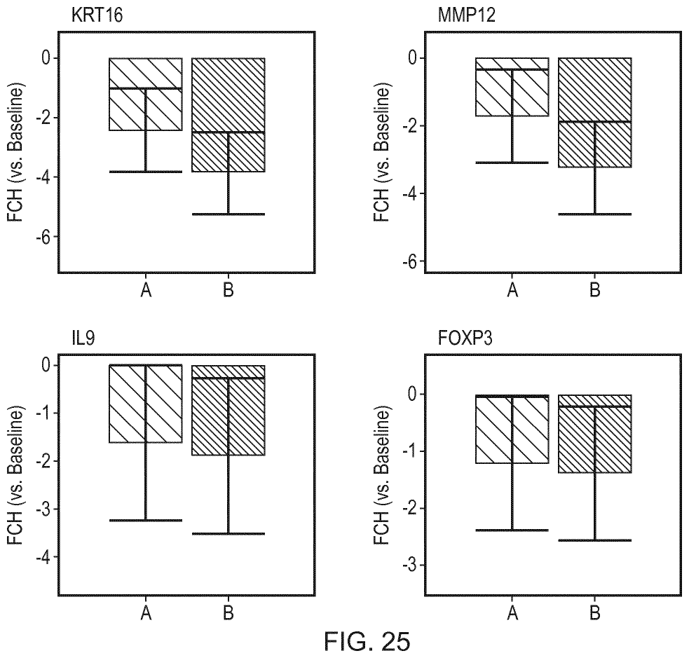

FIG. 25 shows changes in biomarkers (KRT16) associated with proliferation, general inflammation (MMP12), Th9 (IL9) and T regulatory cells (FOXP3).

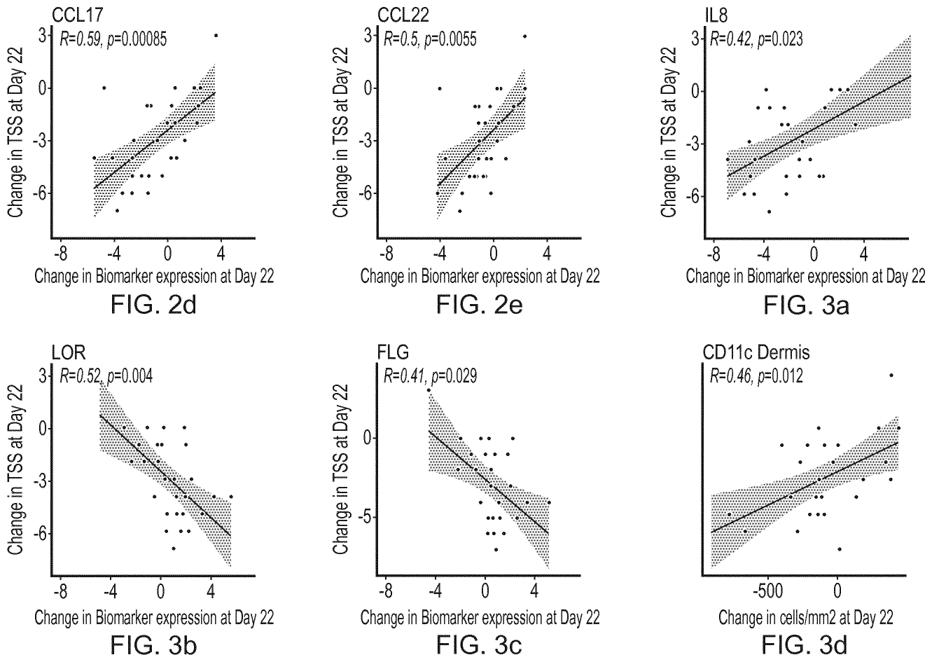

Correlations between change in biomarker expression versus TSS are shown in FIGS. 2-5. The graphs show the correlation of biomarker change at Day 22 compared to baseline to change in TSS at Day 22.

FIGS. 2a and 2b show biomarkers (KRT16, MMP12) associated with proliferation/general inflammation.

FIGS. 2c, 2d and 2e show biomarkers (IL13, CCL17, CCL22) associated with Th2 related chemokines and cytokines.

FIG. 3a show biomarkers (IL8) associated with innate immunity.

FIGS. 3b and 3c show biomarkers (LOR, FLG) associated with skin barrier/terminal differentiation.

FIG. 3d show biomarkers (CD11c Dermis) associated with dendritic cells.

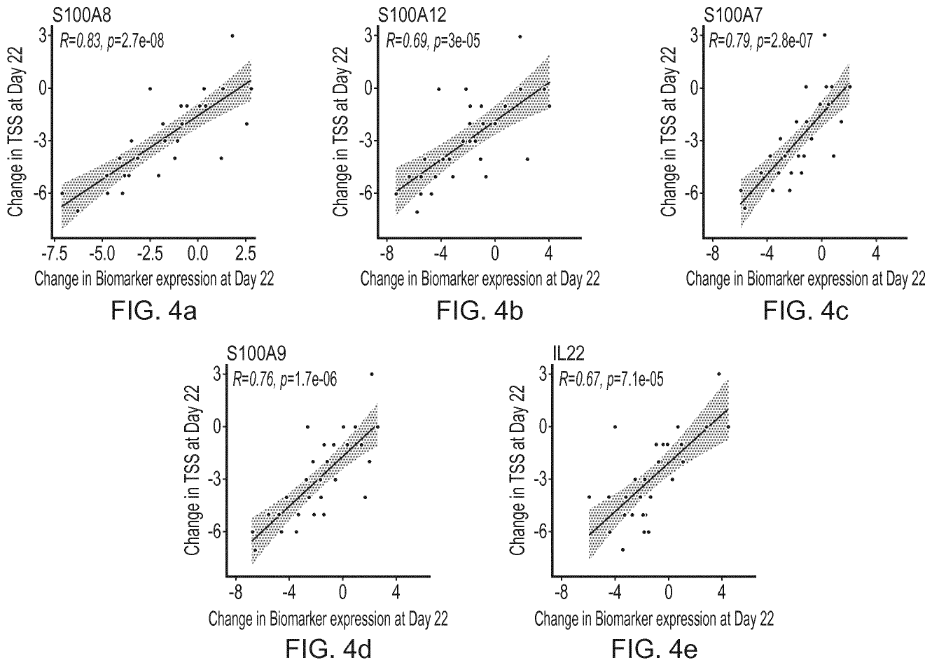

FIGS. 4a-4e show biomarkers (S100A8, S100A12, S100A7, S100A9, IL22) associated with Th17/Th22 related chemokines and cytokines.

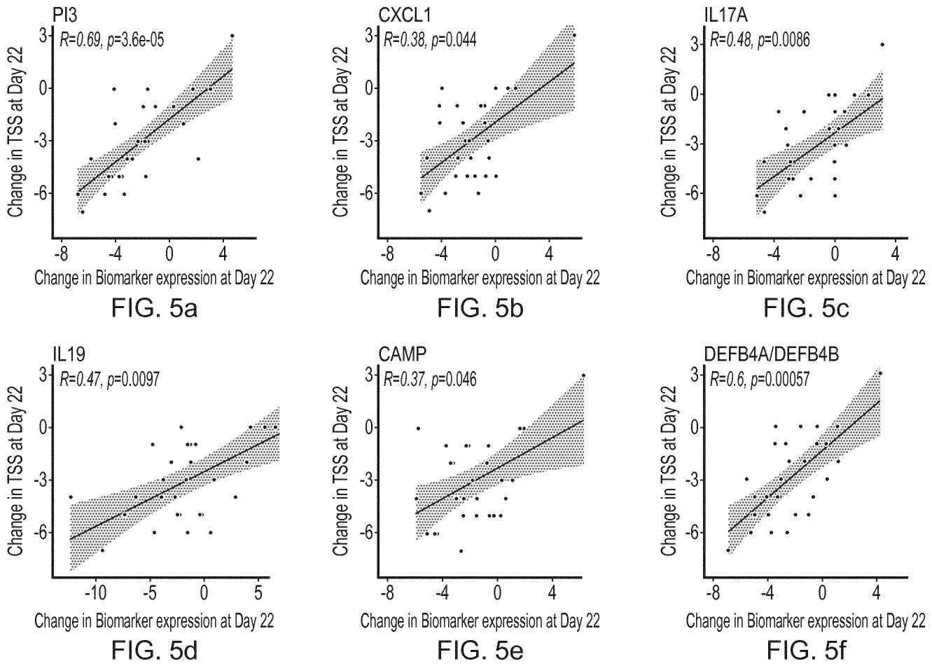

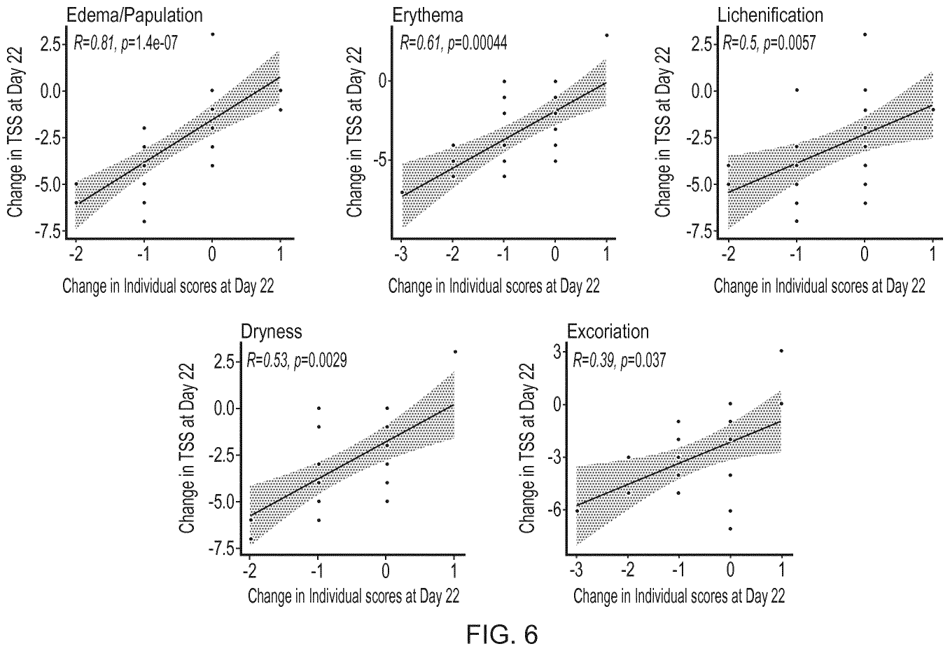

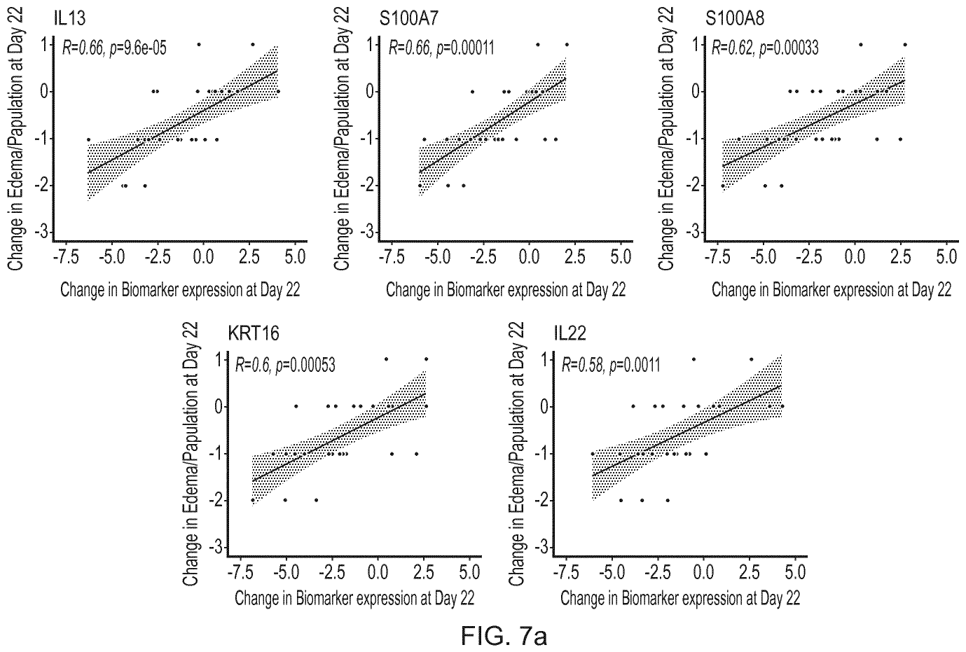

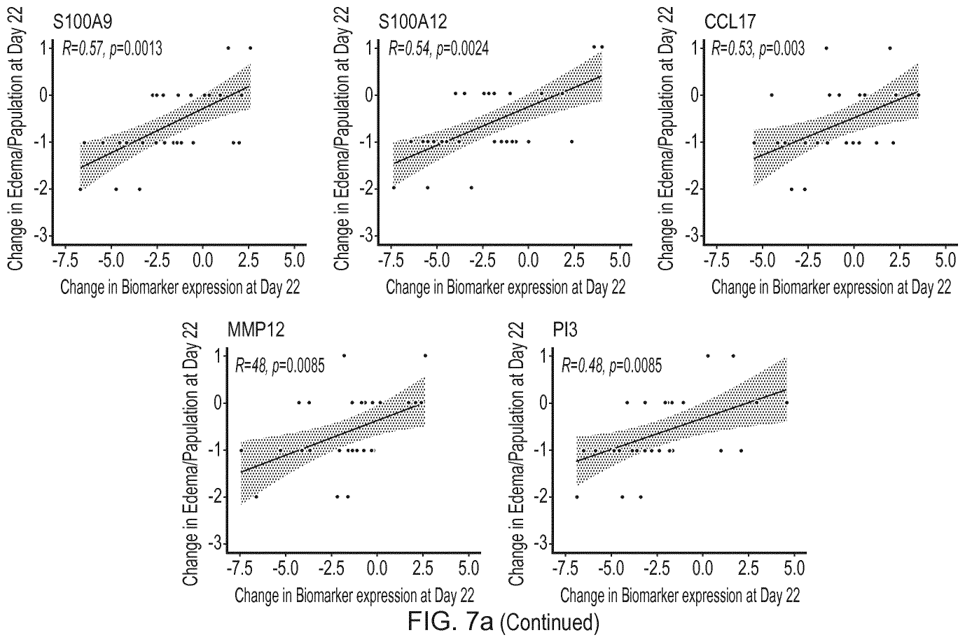

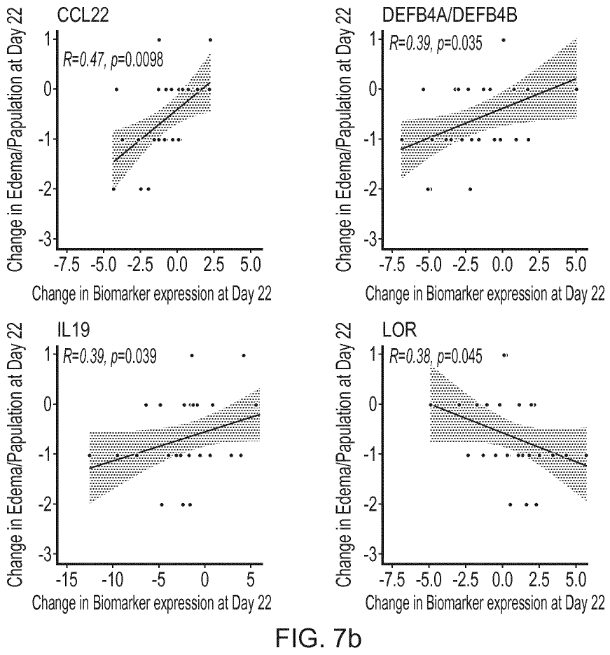

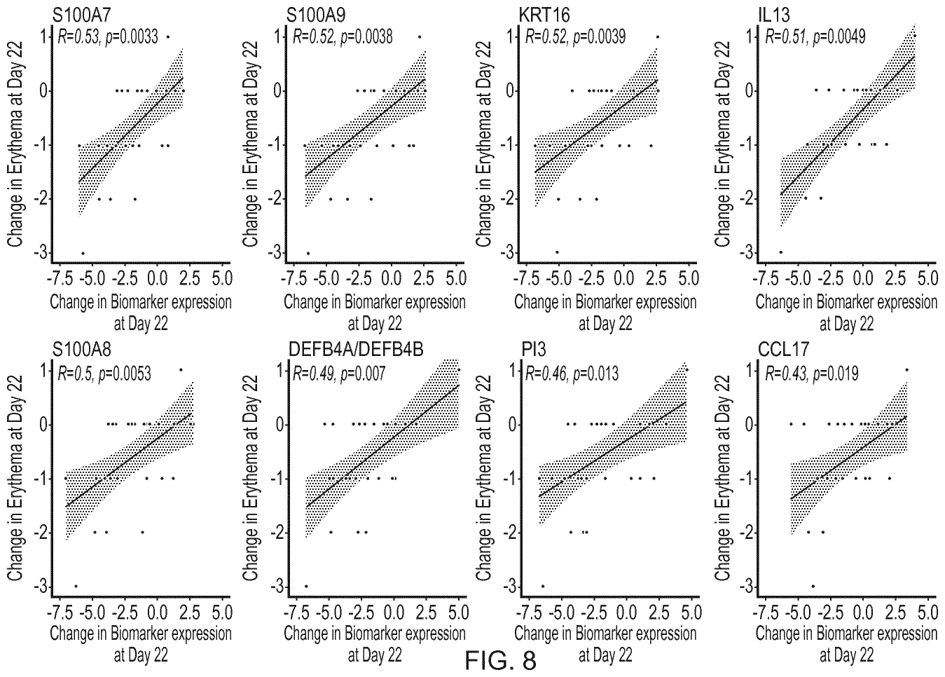

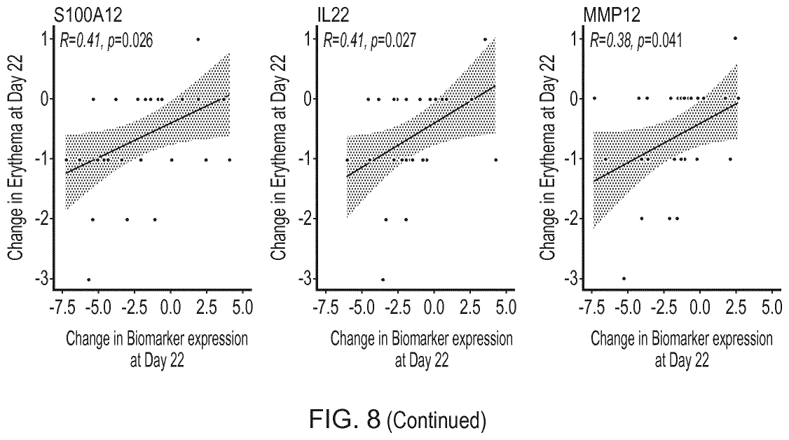

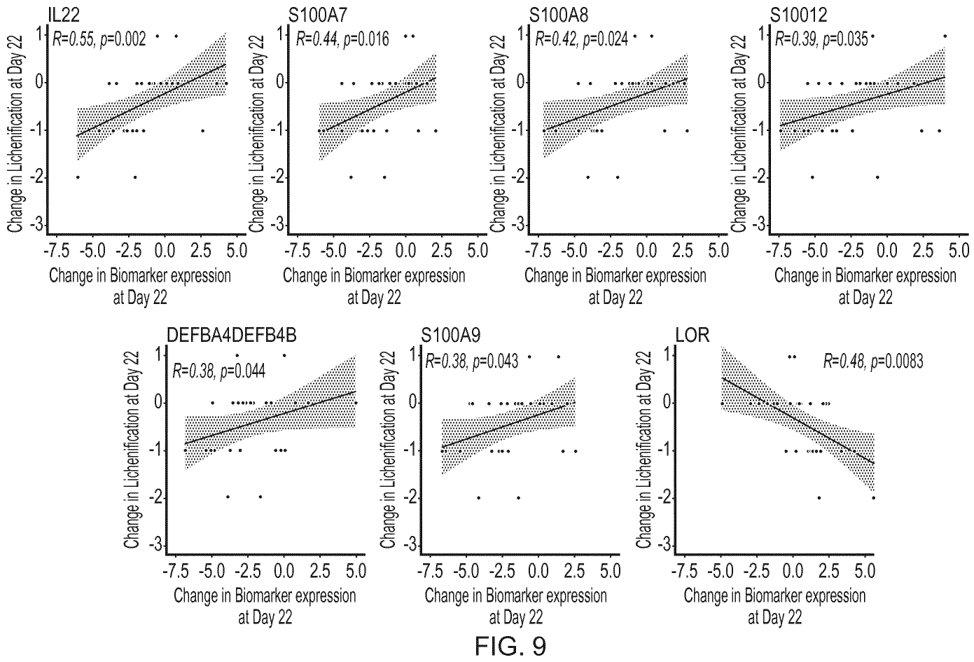

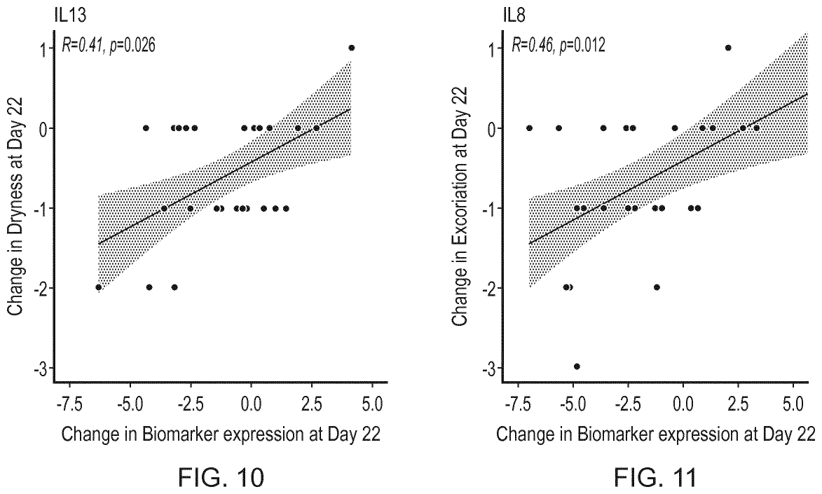

FIGS. 5a-5f show biomarkers (PI3, CXCL1, IL17A, IL19, CAMP, DEFB4A/DEFB4B) associated with Th17 related chemokines and cytokines.

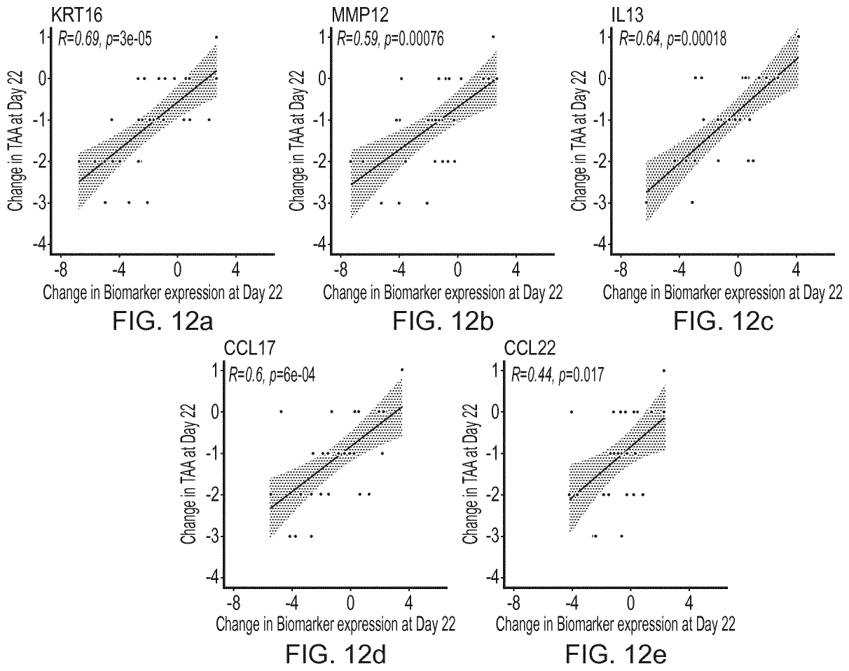

Correlations between change in biomarker expression versus TAA are shown in FIGS. 12-15. The graphs show the biomarker change at Day 22 compared to baseline.

FIGS. 12a and 12b show biomarkers (KRT16, MMP12) associated with proliferation/general inflammation.

FIGS. 12c, 12d and 12e show biomarkers (IL13, CCL17, CCL22) associated with Th2 related chemokines and cytokines.

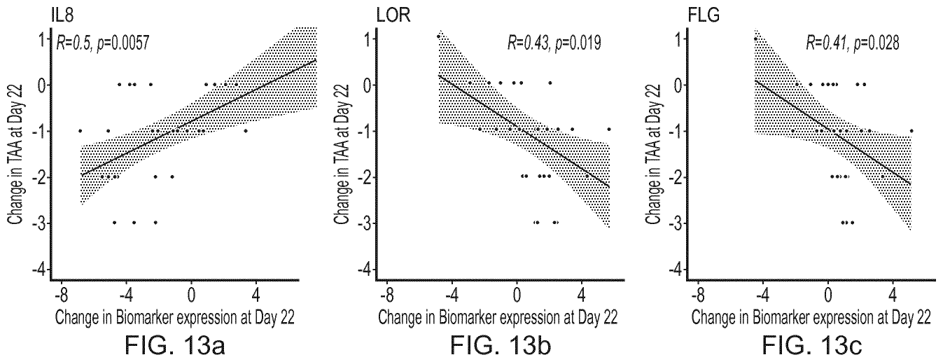

FIG. 13a show biomarkers (IL8) associated with innate immunity.

FIGS. 13b and 13c show biomarkers (LOR, FLG) associated with skin barrier/terminal differentiation.

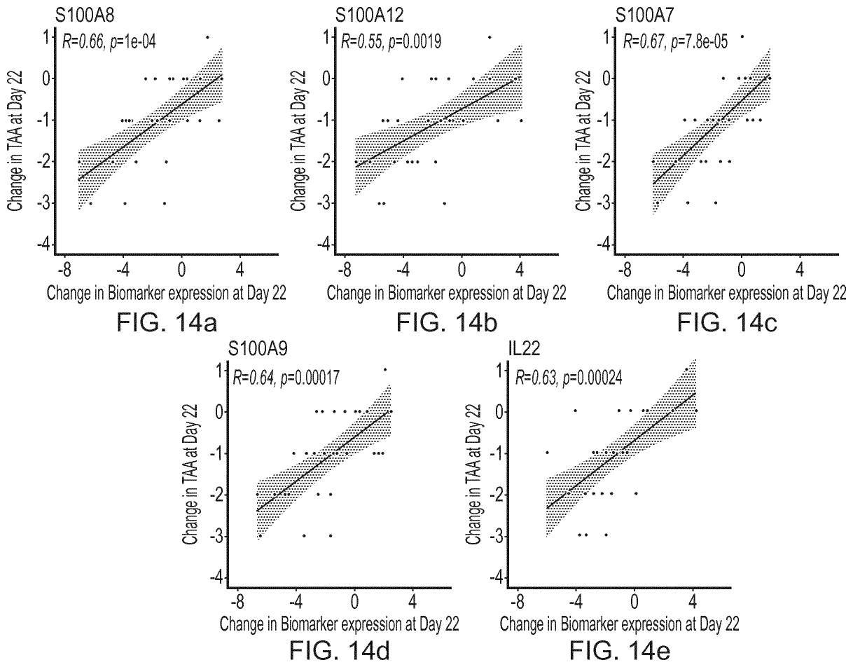

FIGS. 14a-14e show biomarkers (S100A8, S100A12, S100A7, S100A9, IL22) associated with Th17/Th22 related chemokines and cytokines.

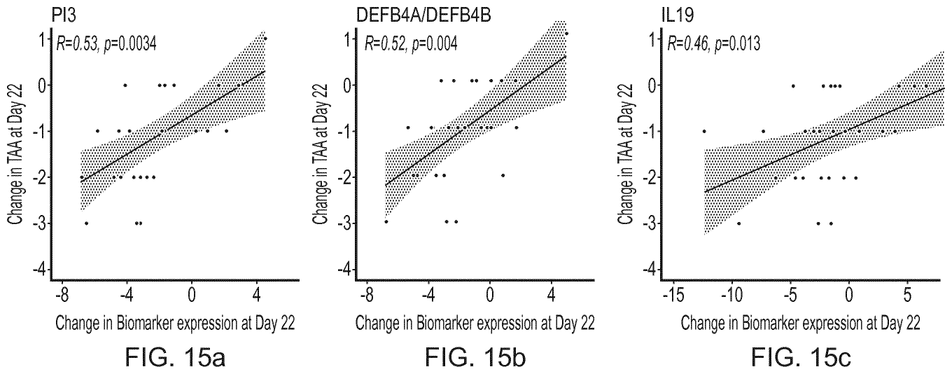

FIGS. 15a-15c show biomarkers (PI3, DEFB4A/DEFB4B, IL19) associated with Th17 related chemokines and cytokines.

All these biomarkers analyzed with qRT-PCR except for LOR and FLG were found to have decreased significantly at Day 22 following topical administration of 2% niclosamide compared to baseline (see Tables 8-13).

LOR and FLG were found to have increased significantly at Day 22 following topical administration of 2% niclosamide compared to baseline, see FIGS. 13b and 13c. LOR and FLG are involved in terminal differentiation of epidermal cells and an increased expression of any one of these proteins is associated with a better skin barrier. Increased expression of LOR induced by topical niclosamide was shown to be associated with an improvement of signs and symptoms of AD.

Also, some skin barrier proteins and lipids analyzed with microarray (see Tables 14-16) were found to have increased significantly at Day 22 following topical administration of 2% niclosamide compared to baseline and vehicle. Skin barrier lipids that were found to have increased compared to baseline and vehicle, by using the microarray analysis, were ACOX2, EVOLV3, FA2H, FAR2, KRT79, PNPLA3. Skin barrier proteins that were found to have increased compared to baseline and vehicle, by using the microarray analysis, were DGAT2 and FAXDC2.

The increased expression of structural skin barrier proteins and lipids indicate that niclosamide are useful for treatment of an inflammatory skin condition associated with skin barrier dysfunction, e.g. an inflammatory skin condition associated with skin barrier deficiency in one or more skin barrier molecules, such as AD, by improving the skin barrier function.

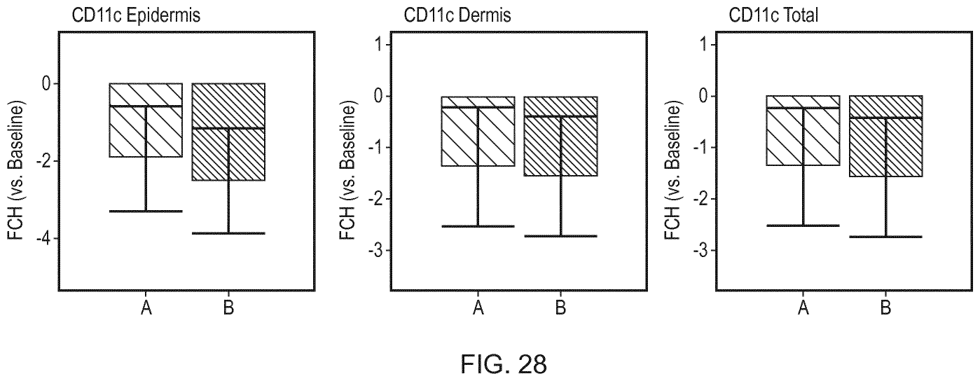

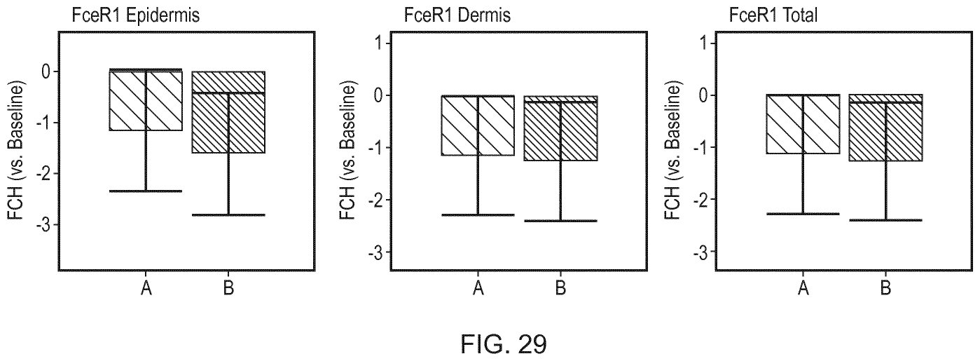

Treatment with 2% niclosamide was shown to decrease inflammation and immune cell infiltrates compared to baseline (pre-dosing at Day 1). Significant reductions in inflammatory cells (dendritic cells: CD11c, FceR1 in epidermis, and Langerhans cells: langerin/CD207) compared to baseline (pre-dosing at Day 1) in patients topically treated with 2% niclosamide were found (FIGS. 27-29). CD11c Dermis was significantly changed in expression level compared to baseline and clinically correlated to TSS (see FIG. 28).

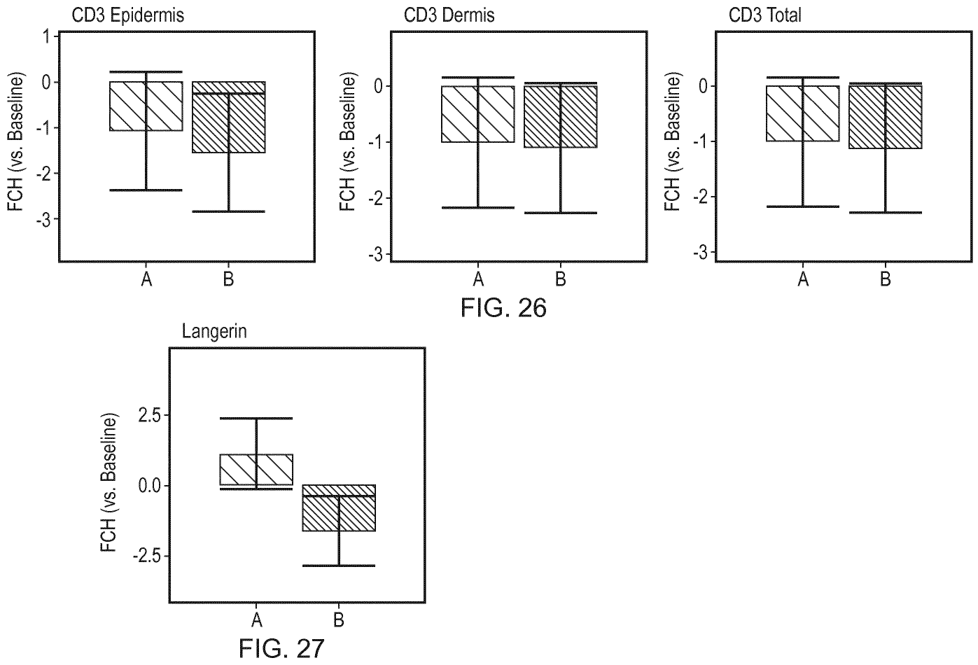

No significant change of the total amount of T cells (i.e. T cells expressing CD3D and CD3G) was found (in dermis and epidermis) compared to baseline (pre-dosing at Day 1) in patients topically treated with 2% niclosamide, see FIG. 26.

In patients treated with 2% niclosamide, there were significant changes from baseline in certain inflammatory markers including those of general inflammation (MMP12), proliferation (KRT16), innate immunity (IL6, IL170, IL8, IL1B), terminal differentiation (FLG, LOR), T-Cell/NK cell activation (IL15, IL15RA), Th1 pathway (CXCL10), Th2 pathway (CCL17, CCL18, CCL22, IL10, IL13, IL5, TSLPR), Th17 pathway (IL17A, IL23p19, IL23A, CCL20, CXCL1, CXCL2, PI3, DEFB4A/DEFB4B, PI3, IL12B), general inflammation (MMP12), T regulatory cells (FOXP3), Th17/TH22 pathway (S100A7, IL22, S100A8, S100A9, S100A12).

The results show that topical administration of niclosamide significantly downregulates expression of immune effectors associated with the Th1, Th2, Th17 and Th22-type immune responses, including innate immune effectors.

Th2, Th17, Th22 responses are crucial in the inflammatory loop of AD. The reduced expression of these key biomarkers and the direct correlation of these biomarkers to clinical signs and symptoms strongly support use of niclosamide for treatment of AD.

Brunner et al (The Journal of Allergy and Clinical Immunology, Volume 139, Issue 4, Supplement, Pages S65-S76, 2017) discloses the effects of dupilumab on lesional AD skin, such as reduction in expression of Th2-associated molecules, such as CCL17, CCL18, and CCL26, and decrease in mediators associated with T_{H}17 and T_{H}22 responses. The biomarker profile in lesional AD skin treated with dupilumab is shown in FIG. 4 of the Brunner reference.

Reference is here also made to Hamilton, Jennifer D., et al. “Dupilumab improves the molecular signature in skin of patients with moderate-to-severe atopic dermatitis.” Journal of Allergy and Clinical Immunology 134.6 (2014): 1293-1300; and Brunner, Patrick M., et al. “A mild topical steroid leads to progressive anti-inflammatory effects in the skin of patients with moderate-to-severe atopic dermatitis.” Journal of Allergy and Clinical Immunology 138.1 (2016): 169-178.

### Example 4: Exploratory Pharmacokinetic Study in Dogs

A preliminary PK study was carried out in dogs in which oxyclozanide was orally administered in the following treatment arms: 5 mg/kg orally once per day for 4 consecutive days; topically administered as a single 20 mg/kg (using Composition K described in Example 5 below); and intravenously as a single 2.5 mg/kg dose.

Oxyclozanide was distributed according to the two-compartment model following intravenous administration. Terminal half-life was 38 h and Vss was about 0.60 L/kg suggesting a low to moderate volume of distribution. Bioavailability was 48% following oral administration and 12% after topical application suggesting a small systemic exposure. Oxyclozanide accumulated well in stratum corneum following topical application for a long period of time when tape stripping (adhesive films) were collected at about 8 cm of the application site (along the backbone).

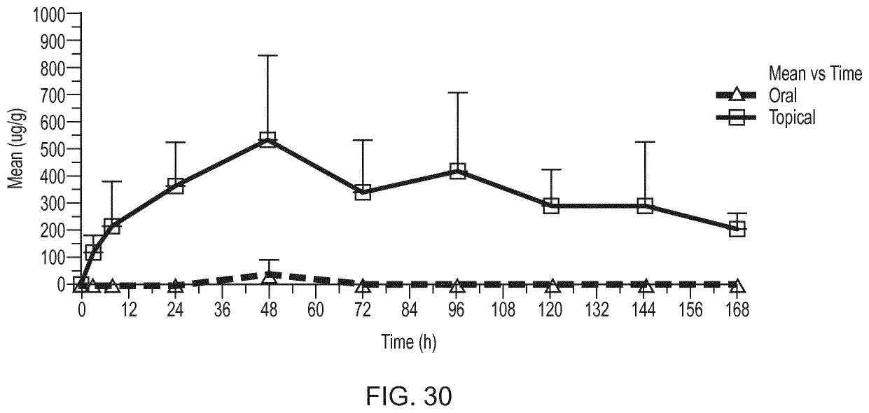

FIG. 30 illustrates the arithmetic profile of oxyclozanide (μg/g) in stratum corneum of skin flank following the oral or topical administration in dogs.

### Example 5: Dermatopharmacokinetic Study Oxyclozanide in Plasma and Skin Following Topical Administration to Dogs

### Study Objective

The aim of this study was to determine on dog (i) the tolerance and pharmacokinetic profile in plasma and skin of oxyclozanide after topical application of Composition K, and (ii) the spread of oxyclozanide on several areas of the skin 10-40 cm away from the application site following topical application of composition K for 4 weeks.

| Composition K | Grams per 100 mL |
|---|---|
| Oxyclozanide | 10 |
| dimethylsulfoxide (DMSO) | 46 |
| diethylene glycol monoethyl ether (Transcutol ®) | q.s. |

### Inclusion Criteria

12 adult beagle dogs having a body weight of more than 11 kg were used in the study. An inclusion clinical examination was performed on D-3ₐ (i.e. 3 days before first administration) and D-4_{b} (i.e. 4 days before first administration) of each phase. Twelve healthy dogs with haematological-biochemical parameters within the supplier's normal range were included in the study.

### Management of Test System

During the 28-days animal study phase (from D0 to D28), the animals were housed individually in order to guarantee the quality of the results by avoiding the dogs licking each other after the treatment is applied. No treatment, apart from those treatments included under the study, was administered without the approval of the study director.

### Treatment

The cutaneous route (topical administration) was the route of administration for composition K on dogs. Composition K was applied evenly along the backbone starting from the base of the tail to the back of the neck. The administered dosage was 20 mg oxyclozanide/kg (0.20 ml/kg of body weight).

The 12 dogs were divided into 2 groups of 6 animals, with homogeneous averages of age and weight. They were treated with the same composition but the activities following administration were different:

### Group A: Dogs 1 to 6

Blood samplings
Skin biopsies, preceded by cutaneous cells (stratum corneum) samplings at the belly zone
Cosmetic and dermal assessments

### Group B: Dogs 7 to 12

Cutaneous cells (stratum corneum) samplings at several sites (ear, shoulder, belly, fore leg, hock joint and chest zones).

The volumes administered for each dog are described in the table below:

| | | | Amount of | |
|---|---|---|---|---|
| | | | oxyclozanide | |
| | Bodyweight | Administered | administered | Dose |
| Dog No. | (kg) | volume (mL) | (mg) | (mg(kg) |
| 1 | 13.1 | 2.6 | 260 | 19.8 |
| 2 | 11.5 | 2.3 | 230 | 20.0 |
| 3 | 12.0 | 2.4 | 240 | 20.0 |
| 4 | 10.1 | 2.0 | 200 | 19.8 |
| 5 | 12.4 | 2.5 | 250 | 20.2 |
| 6 | 14.4 | 2.9 | 290 | 20.1 |
| 7 | 17.3 | 3.5 | 350 | 20.2 |
| 8 | 17.4 | 3.5 | 350 | 20.1 |
| 9 | 17.3 | 3.5 | 350 | 20.2 |
| 10 | 15.6 | 3.1 | 310 | 19.9 |
| 11 | 15.9 | 3.2 | 320 | 20.1 |
| 12 | 15.9 | 3.2 | 320 | 20.1 |

### Clinical Follow-Up

On D-3a or D-4b, a physical examination, including a weigh-in, was performed on the 6 dogs of each group by a qualified person.

On D0 of each phase, in the hour following treatment administration, a clinical observation was performed. Special attention was given to the following clinical signs: constitutional symptoms (such as fever, fatigue, shivering), neurological and vascular, ptyalism, vomiting, pruritus, pain, unusual behaviour (e.g. rolling on back).

All dogs in group A were shaved at D-3a in the belly zone (lower part of the abdomen to the right or left of the animal) on sufficient areas for biopsies. Shaving was repeated at D4a, D11a, D18a and D25a alternating the right and left side of the animal for each sampling time.

All dogs of group B were shaved on D-4b at the following areas, alternating the right and left side of the animal for each sampling time: ear, belly, chest, hock (tarsal) joint, fore leg, and shoulder. Each area was shaved on a zone of around 5 cm×5 cm.

Dermal tolerance assessment Dermal tolerance was assessed for the 6 dogs of group A on: D0a+1h, D0a+3h, D0a+24h, D3a, D7a, D14a, D21a and D28a.

Upon each assessment, dermal tolerance in the treatment area was assessed according to the following scoring system:
Erythema:
   No erythema
   Very mild erythema (barely visible)
   Clearly-defined erythema
   Moderate erythema
   Severe erythema (beetroot red) with slight sores (deep lesions)
Oedema (Yes/No)
Excoriation (Yes/No)
Scabs (Yes/No)

### Sampling

### Dry Tube Blood Sampling

Before the inclusion examination and on D28 of both phases, blood samples were taken from the jugular vein of each dog with a 4 ml dry tube and a 3 ml EDTA K3 tube. These samplings were done for hemato-biochemical analysis.

After centrifugation (around 3500 revolutions/min, for 15 minutes at +4° C.), the serum was collected and then divided into two equal aliquots in tubes (Nunc 1.8 ml type). Each aliquot was identified with the study code; the identification number of animal and its case number; the type, date and time of sampling. The aliquots were stored at 5° C.+/−3° C. until their shipping. The aliquots were shipped on the day of sampling, in cold packaging, to the analytical laboratory.

The laboratory analysed the following:
Biochemical parameters: total bilirubin, total protein, glucose, alkaline phosphatase (ALP), alanine aminotransferase (ALAT), aspartate aminotransferase (AST), creatine kinase (CK) and gamma glutamyl transferase (GGT).
Haematological parameters: haemoglobin, haematocrit, RBC, MCV, MCH, MCHC and reticulocytes.

### Lithium Heparin Blood Sampling

For all animals of group A, a blood sample of around 4 ml was collected from the jugular vein using tubes containing lithium heparin at the following times (with tolerance of 10%): D-3a, D0a+1h, D0a+6h, D0a+12h, D0a+16h, D0a+24h, D0a+32h, D0a+48h, D3a, D5a et D7a.

Centrifugation (around 3500 revolutions/min, for 15 minutes at +4° C.) was performed a maximum of 30 minutes after sampling. The plasma was collected and then divided into two aliquots in tubes (Nunc 1.8 ml type) as follows:
S1 aliquot: around 0.5 ml
S2 aliquot: the remainder of the plasma (more than 0.5 ml)

The aliquots were stored at −70° C.+/−5° C. until their shipping. The aliquots were shipped at the end of each phase packaged in dry ice to the analytical laboratory.

### Skin Biopsies

For all dogs of group A, a skin biopsy was performed under anaesthesia in the belly zone (alternating the right and left side of the animal for each sampling time) with a 4 mm ‘Biopsy-Punch’ at the following times: D1a, D7a, D14a, D21a and D28a. Prior to each biopsy a cutaneous cell sampling was performed on the area of the biopsy. The vials were frozen at −70° C.+/−5° C. until shipping for analysis.

### Stratum Corneum Sampling (D-Squame Discs)

Tape-stripping (adhesive films) was carried out in an analogous method to that described in Emilie Vidémont, et al., (“Characterization of the canine skin barrier restoration following acute disruption by tape stripping,” Veterinary Dermatology, vol. 23, pp. 103-123, 2011) and Lionel Trottet (Dermal Drug Selection and Development, An Industrial Perspective. Springer 2017. Page 57). Tape was pressed onto the surface of the skin with a fixed amount of pressure before removal. The superficial layers of the SC which adhere to the film were stripped from the Stratum corneum which was then accessible for further investigation.

For all animals of group B, cutaneous cell sampling was performed on fasted animal on the six shaved zones using discs (D-Squame DISCS), at the following times: D-4b, D0b+24h, D0b+24h, D3b, D7b, D14b, D21b and D28b alternating the right and left side of the animal. 20 discs (D-Squame Discs) of 22 mm-diameters were applied in succession on the target area, in the same zone defined with a marker.

The discs were applied as follow:
Tweezers were used to carefully remove the discs from its backing using the edge provided.
The disc was applied to the defined area.
The disc was pressed for 1 second with D-Squame Pressure Instrument
150 g/cm² and 1 mg of stratum corneum was removed after each disc application. 20 discs were pressed per sample corresponding to removal of 20 mg of stratum corneum per sampling time.

For each cycle, the 20 discs removed from the skin were divided into 2 samples (51 and S2) as follows:
S1: the first 10 discs were placed in a scintillation vial
S2: the last 10 discs were placed in a scintillation vial

The pigmentation of the skin of the sampled area was recorded in raw data. The vials were frozen at −70° C.+/−5° C. until shipping to the analytical laboratory.

### PK Analysis

The pharmacokinetic analysis was performed using Phoenix software (version 6.3, Pharsight, USA). Data points indicated as “missing” were systematically ignored during the calculation and therefore have no effect on the results. A missing status is assigned and no flag symbol is used. Values were excluded if, in the judgement of the pharmacokineticist, they were deemed not to be “pharmacodynamically relevant.” If outliers were suspected, they were identified using the Outlier identification procedure of STATGRAPHICS.

The following pharmacokinetic parameters were evaluated:

| | |
|---|---|
| Cmax | Maximum observed concentration, occurring at Tmax |
| Tmax | Time of maximum observed concentration |
| Lambda_z | First order rate constant associated with the |
| | terminal (log-linear) portion of the curve. Estimated |
| | by linear regression of time vs. log concentration |
| HL_Lambda_z | Terminal half-life |
| Tlast | Time of last measurable (positive) concentration |
| Clast | Concentration corresponding to Tlast |
| AUClast | Area under the moment curve from the time of dosing |
| | (Dosing_time) to the last measurable concentration |
| AUCinf | AUC from Dosing_time extrapolated to infinity, |
| | based on the last observed concentration (obs) |
| AUC_%_Extrap | Percentage of AUCINF_obs due to extrapolation from |
| | Tlast to infinity |
| Cl_F | Total body clearance for extravascular administration |
| Vz_F | Volume of distribution based on the terminal phase |
| Vss | Volume of distribution at steady state |

### Statistical Analysis

Mean (arithmetic average), standard deviation (SD), standard error of the mean, coefficient of variation (CV %, SD/mean*100), maximum value and minimum value were calculated per day and treatment for each concentration parameter. The mean concentrations and the standard deviations were calculated and the mean concentration time curves were plotted. This descriptive statistics of the pharmacokinetic parameters were calculated per treatment.

### Analytical Methods

The oxyclozanide concentration in samples was quantified using an LC-MS-MS method.

### Results

The dogs' general health condition was satisfactory and comparable all along the study. Their results of haematological-biochemical analysis were acceptable without any indication of possible negative effect of the active substance on the parameters examined for the animals of group A. For the animals of group B, there were some anomalies in the haematological-biochemical parameters such as an increase of alanine aminotransferase. This is due to close and repeated anaesthesia for more than 4 weeks by tiletamine metabolized in the liver.

No symptoms or abnormalities were observed after treatment on D0a. After treatment on D0b, two dogs (dogs 9 and 12) licked the base of their tail and they shook themselves. Some slight scabs were observed on the left shoulder (area of skin sampling) on one dog (dog 12) on D0b.

After the cutaneous treatment, no significant sign appeared for dermal tolerance all along the phase A. On the six dogs of the group A, the fur was greasy appearance at D0a+1h and D0a+3h with an orange coloration on light skin for 3 dogs.

### Plasma Samples

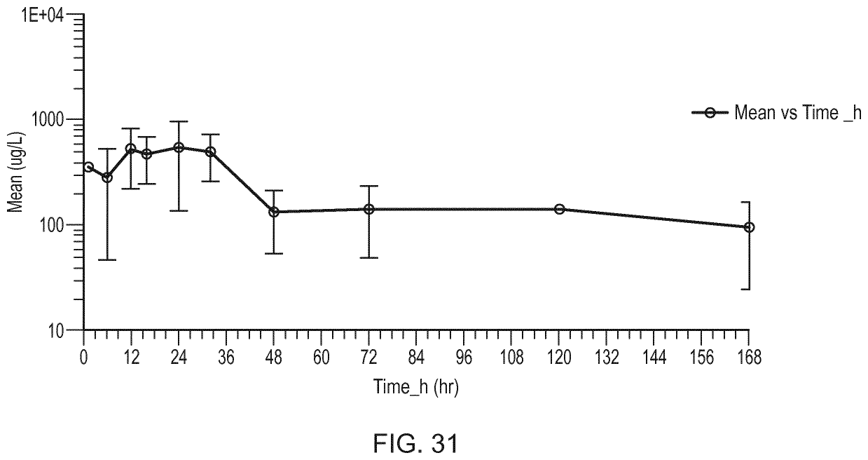

FIG. 31 illustrates the mean plasma concentration-time of oxyclozanide (μg/L) obtained following topical administration in dogs.

Oxyclozanide presents a PK profile in plasma similar to those obtained in the early PK study of Example 4 and at same systemic exposition. Bioavailability was not calculated because there are not intravenous route in this study. However, bioavailability appeared to be similar (about 12%) to that the exploratory pharmacokinetics study of Example 4, when comparing AUC_{inf} (60604 h*μg/L against 55712 h*μg/L). Low concentrations of oxyclozanide in plasma were observed after topical administration suggesting a low systemic exposure.

### Epidermis/Dermis (Biopsies)+Stratum Corneum (Strip Samples)

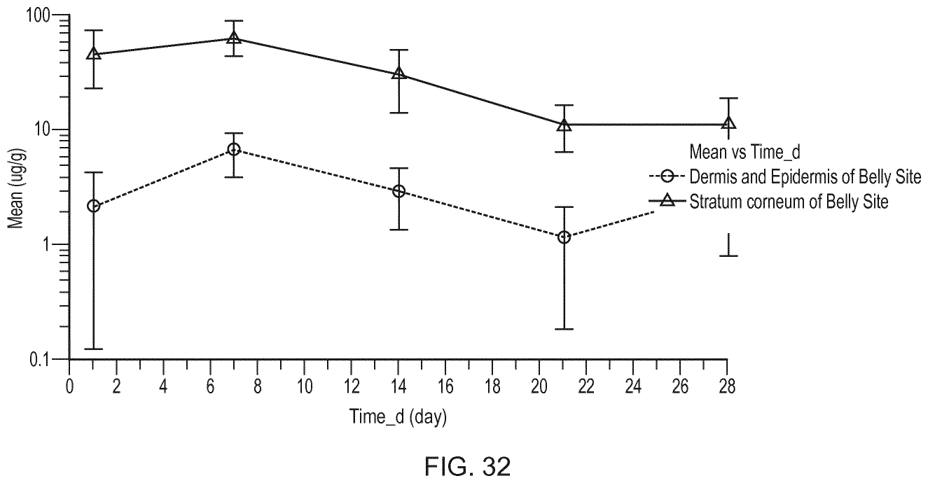

FIG. 32 illustrates the mean (μg/g) skin biopsies concentration-time of oxyclozanide obtained following topical administration in dogs.

Oxyclozanide accumulates about 10 fold less in deep skin (epidermis and dermis) than in the stratum corneum. The inter-variability between animals was low after topical application.

Table 17 below shows the mean exposure (AUC) of each collected site. Exposure of oxyclozanide in the superficial skin and deep skin was observed for a prolonged period of time after administration.

TABLE 17

| | AUC | AUC | AUC | AUC |
|---|---|---|---|---|
| | 7 days | 14 days | 21 days | 28 days |
| Area_sites | (day*μg/g) | (day*μg/g) | (day*μg/g) | (day*μg/g) |
| Dermis and | 28 | 61 | 76 | 91 |
| Epidermis of | | | | |
| Belly site | | | | |
| Stratum | 362 | 701 | 852 | 932 |
| corneum of | | | | |
| Belly site | | | | |

### Stratum Corneum (Strip Samples)

Oxyclozanide accumulates well in stratum corneum following topical application for a long period of time as observed in the previous study of Example 4. Oxyclozanide diffuse well on all the skin area zones of the body and stay significantly above the MICs although it is difficult to compare concentrations of oxyclozanide solubilized in skin lipids and measurement of the MIC determined in a buffered aqueous medium.

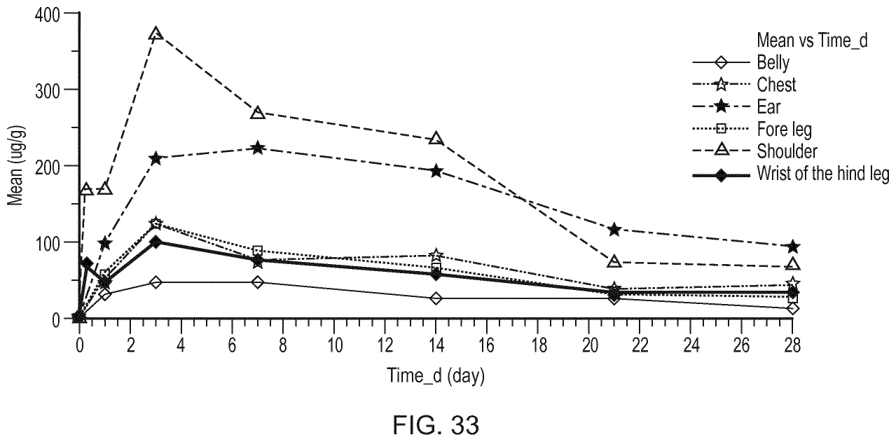

FIG. 33 illustrates mean (μg/g) stratum corneum (strips) concentration-time of oxyclozanide obtained following topical administration in dogs on 6 zones.

Results show that concentrations of oxyclozanide were quite correlated with the distance between the site of administration and the sampling site of skin collection.

Oxyclozanide is slowly eliminated from the superficial skin (stratum corneum) and elimination rate seems correlated to the process of cell migration through the layers of the epidermis (epidermal renewal) takes approximately 22-28 days.

Table 18 shows the mean exposure (AUC) of each collected site. Exposure of oxyclozanide in the superficial skin correlated with the distance between the site of administration and the sampling site of skin collection for several period of time after administration.

TABLE 18

| | AUC | AUC | AUC | AUC |
|---|---|---|---|---|
| | 7 days | 14 days | 21 days | 28 days |
| Area_sites | (day*μg/g) | (day*μg/g) | (day*μg/g) | (day*μg/g) |
| Belly | 291 | 560 | 752 | 901 |
| Chest | 593 | 1140 | 1527 | 1785 |
| Ear | 1231 | 2684 | 3756 | 4488 |
| Fore leg | 638 | 1172 | 1513 | 1729 |
| Shoulder | 1965 | 3722 | 4803 | 5305 |
| Hock joint | 565 | 1042 | 1375 | 1617 |

### Conclusions

All twelve dogs remained in good general condition during the study and no significant signs of dermal intolerance were observed.

High concentrations of oxyclozanide were observed in the stratum corneum for a prolonged period in all the skin area zones of the body. The distribution of oxyclozanide between the superficial and deep skin was about 10:1. Low systemic concentrations were observed as in the earlier pharmacokinetic study of Example 4.

### Further Embodiments

Further embodiments of the invention are set out in the following numbered clauses:
1. A halogenated salicylanilide, or a pharmaceutically acceptable salt or hydrate thereof, for use in the treatment of dermatitis in a non-human subject to reduce or eliminate one or more of pruritus, erythema, induration, excoriation, lichenification, scaling, oozing, crusting, xerosis, exfoliation, lesion nodules, prurigo nodules, lesion vesicles, lesion papules, lesion plaques or lesion swelling associated with the dermatitis.
2. The halogenated salicylanilide for the use of clause 1, wherein the dermatitis is severe dermatitis.
3. The halogenated salicylanilide for the use of clause 1, wherein the dermatitis is moderate dermatitis.
4. The halogenated salicylanilide for the use of clause 1, wherein the dermatitis is mild dermatitis.
5. The halogenated salicylanilide for the use of clause 1, wherein the dermatitis is moderate to severe dermatitis.
6. The halogenated salicylanilide for the use of clause 1, wherein the dermatitis is mild to moderate dermatitis.
7. The halogenated salicylanilide of any of clauses 1 to 6, for use in the treatment of an exacerbation of dermatitis.
8. The halogenated salicylanilide for the use of any of clauses 1 to 7, wherein the dermatitis is an acute form of dermatitis.
9. The halogenated salicylanilide for the use of any of clauses 1 to 7, wherein the dermatitis is a chronic form of dermatitis.
10. The halogenated salicylanilide for the use of any of clauses 1 to 9, wherein the dermatitis is selected from topic dermatitis, contact dermatitis, allergic contact dermatitis, irritant contact dermatitis, atopic dermatitis, seborrhoeic dermatitis, actinic dermatitis, pododermatitis, demodicosis, pompholyx dermatitis, lichen simplex chronicus (including Canine acral lick dermatitis and neurodermatitis), digital dermatitis (including bovine digital dermatitis), exfoliative dermatitis (drythroderma), carcinomatous dermatitis, nummular dermatitis, stasis dermatitis, flea allergy dermatitis, otitis, food allergic dermatitis, malassezia dermatitis, intertrigo, perioral dermatitis, dermatomyositis, eczematous dermatitis, photoallergic dermatitis, phototoxic dermatitis, phytophotodermatitis or radiation-induced dermatitis.
11. The halogenated salicylanilide for the use of any of clauses 1 to 9, wherein the dermatitis is atopic dermatitis.
12. A method for the treatment of dermatitis in a non-human subject to reduce or eliminate one or more of pruritus, erythema, induration, excoriation, lichenification, scaling, oozing, crusting, xerosis, exfoliation, lesion nodules, prurigo nodules, lesion vesicles, lesion papules, lesion plaques or lesion swelling associated with the dermatitis, the method comprising administrating to the subject an effective amount of a halogenated salicylanilide, or a pharmaceutically acceptable salt or hydrate thereof.
13. The method of clause 12, wherein the dermatitis is severe dermatitis.
14. The method of clause 12, wherein the dermatitis is moderate dermatitis.
15. The method of clause 12, wherein the dermatitis is mild dermatitis.
16. The method of clause 12, wherein the dermatitis is moderate to severe dermatitis.
17. A method for the treatment of an exacerbation of dermatitis in a subject, the method comprising administrating to the subject an effective amount of a halogenated salicylanilide, or a pharmaceutically acceptable salt or hydrate thereof.
18. The method of clause 17, wherein the treatment reduces or eliminates one or more pruritus, erythema, induration, excoriation, lichenification, scaling, oozing, crusting, xerosis, lesion nodules, prurigo nodules, lesion vesicles, lesion papules, lesion plaques or lesion swelling, associated with the dermatitis.
19. The method of any of clauses 12 to 18, wherein the atopic dermatitis is acute dermatitis.
20. The method of any of clauses 12 to 18, wherein the atopic dermatitis is chronic dermatitis.
21. The method of any of clauses 12 to 20, wherein the dermatitis is selected from topic dermatitis, contact dermatitis, allergic contact dermatitis, irritant contact dermatitis, atopic dermatitis, seborrhoeic dermatitis, actinic dermatitis, pododermatitis, demodicosis, pompholyx dermatitis, lichen simplex chronicus (including Canine acral lick dermatitis and neurodermatitis), digital dermatitis (including bovine digital dermatitis), exfoliative dermatitis (drythroderma), carcinomatous dermatitis, nummular dermatitis, stasis dermatitis, flea allergy dermatitis, otitis, food allergic dermatitis, malassezia dermatitis, intertrigo, perioral dermatitis, dermatomyositis, eczematous dermatitis, photoallergic dermatitis, phototoxic dermatitis, phytophotodermatitis or radiation-induced dermatitis.
22. The method of any of clauses 12 to 20, wherein the dermatitis is atopic dermatitis
23. The halogenated salicylanilide for the use of any of clauses 1 to 11, or the method of any of clauses 12 to 22, wherein the subject is a companion animal, preferably a dog or cat, more preferably a dog.
24. The halogenated salicylanilide for the use of any of clauses 1 to 11, or the method of any of clauses 12 to 22, wherein the halogenated salicylanilide is selected from rafoxanide, oxyclozanide, closantel and niclosamide or a pharmaceutically acceptable salt, solvate or ester thereof.
25. The halogenated salicylanilide for the use of any of clauses 1 to 11, or the method of any of clauses 12 to 22, wherein the halogenated salicylanilide is niclosamide or a pharmaceutically acceptable salt or hydrate thereof, optionally niclosamide or a pharmaceutically acceptable salt thereof, for example wherein the halogenated salicylanilide is niclosamide.
26. The halogenated salicylanilide for the use of any of clauses 1 to 11, or the method of any of clauses 12 to 22, wherein the halogenated salicylanilide is oxyclozanide, or a pharmaceutically acceptable salt or hydrate thereof, optionally oxyclozanide or a pharmaceutically acceptable salt thereof, for example wherein the halogenated salicylanilide is oxyclozanide.
27. The halogenated salicylanilide for the use of any of clauses 1 to 11, or the method of any of clauses 12 to 22, wherein the halogenated salicylanilide is topically administered to the subject.
28. The halogenated salicylanilide for the use or method of clause 27, wherein the halogenated salicylanilide is topically administered in the form of a topical composition.
29. The halogenated salicylanilide for the use or method of clause 28, wherein the topical composition is selected from a topical cream, ointment, gel, paste, foam, solution, suspension, pour-on, spot-on or line-on composition.
30. The halogenated salicylanilide for the use or method of clause 28 or clause 29, wherein the topical composition comprises the halogenated salicylanilide and a formulation base selected from an oleaginous base (e.g. petrolatum, white petrolatum, yellow ointment or white ointment), an absorption base (e.g. hydrophilic petrolatum or lanolin), a water-removable base (oil in water emulsion); and a water-soluble base (e.g. a polyethylene glycol).
31. The halogenated salicylanilide for the use or method of any of clauses 28 to 30, wherein the topical composition is a non-aqueous composition.
32. The halogenated salicylanilide for the use or method of any of clauses 28 to 31, wherein the topical composition is an aqueous composition.
33. The halogenated salicylanilide for the use or method of clause 28 or clause 29, wherein the topical composition is a non-aqueous topical composition comprising:
   (i) the halogenated salicylanilide (for example selected from niclosamide, rafoxanide, oxyclozanide and closantel), or a pharmaceutically acceptable salt or hydrate thereof; and
   (ii) polyethylene glycol (PEG), preferably a PEG with a melting point of less than 40° C.
34. The halogenated salicylanilide for the use or method of clause 28 or clause 29 wherein the topical composition is a non-aqueous topical composition comprising:
   (i) 0.01 to 4.5% (for example 0.1 to 3% or about 2%) by weight of the halogenated salicylanilide, or a pharmaceutically acceptable salt or hydrate thereof; and
   (ii) at least 70% (for example at least 90%) by weight of a PEG, wherein the average molecular weight of the PEG is 600 or less (for example less than 600 or from about 200 to about 600 or about 400).
35. The halogenated salicylanilide for the use or method of clause 28 or clause 29, wherein the topical composition further comprises a non-polymeric glycol (e.g. propylene glycol).
36. The halogenated salicylanilide for the use or method of any of clauses 28 to 35, wherein the halogenated salicylanilide is dissolved or partially dissolved in the composition.
37. A topical composition comprising 2 to 20% wt/v, preferably 5 to 15% wt/v, more preferably, 8 to 12% wt/v of a halogenated salicylnalinide (e.g. niclosamide or oxyclozanide or a pharmaceutically acceptable salt or hydrate thereof);
35 to 55% wt/v (preferably 30 to 50% wt/v more preferably, about 45% wt/v) of a polar aprotic solvent, for example dimethyl sulfoxide (DMSO);
25 to 55% w/v (preferably 30 to 55% wt/v, more preferably 35 to 50% w/v of a glycol ether (e.g. 2-(2-ethoxyethoxy)ethanol);
and 0 to 10% w/v (preferably 0 to 5% w/v (e.g. 0, or 1 to 5% w/v) alkanol amine (e.g. ethanolamine.
38. The topical composition of clause 37, selected from composition is selected from Formulation A′ to I′ shown in the table below:

| | A′ | B′ | C′ | D′ | E′ | F′ | G′ | H′ | I′ |
|---|---|---|---|---|---|---|---|---|---|
| Oxyclozanide (wt/v %) | 8 | 9 | 10 | 12 | 15 | 20 | 2 | 5 | 11 |
| DMSO (wt/v %) | 40 | 42 | 46 | 45 | 45 | 46 | 40 | 40 | 45 |
| Monoethanolamine | 3 | — | — | — | — | 3 | 3 | — | — |
| Transcutol P (QSP) | Qsp | Qsp | Qsp | Qsp | Qsp | Qsp | Qsp | Qsp | Qsp |

39. The topical composition of clause 37 or clause 38, wherein the halogenated salicylanilide is oxyclozanide or a pharmaceutically acceptable salt or hydrate thereof.
40. The topical composition of any of clauses 37 to 39, wherein composition is in the form of a spot-on or line-on composition.

1-11. (canceled)

12. A method for the treatment of dermatitis in a non-human subject to reduce or eliminate one or more of pruritus, erythema, induration, excoriation, lichenification, scaling, oozing, crusting, xerosis, exfoliation, lesion nodules, prurigo nodules, lesion vesicles, lesion papules, lesion plaques or lesion swelling associated with the dermatitis, the method comprising administrating to the subject an effective amount of a halogenated salicylanilide, or a pharmaceutically acceptable salt or hydrate thereof.

13. The method of claim 12, wherein the dermatitis is severe dermatitis.

14. The method of claim 12, wherein the dermatitis is moderate dermatitis.

15. The method of claim 12, wherein the dermatitis is mild dermatitis.

16. The method of claim 12, wherein the dermatitis is moderate to severe dermatitis.

17. A method for the treatment of an exacerbation of dermatitis in a subject, the method comprising administrating to the subject an effective amount of a halogenated salicylanilide, or a pharmaceutically acceptable salt or hydrate thereof.

18. The method of claim 17, wherein the treatment reduces or eliminates one or more pruritus, erythema, induration, excoriation, lichenification, scaling, oozing, crusting, xerosis, lesion nodules, prurigo nodules, lesion vesicles, lesion papules, lesion plaques or lesion swelling, associated with the dermatitis.

19. The method of claim 12, wherein
the atopic dermatitis is acute dermatitis.

20. The method of claim 12, wherein the atopic dermatitis is chronic dermatitis.

21. The method of claim 12, wherein the dermatitis is selected from topic dermatitis, contact dermatitis, allergic contact dermatitis, irritant contact dermatitis, atopic dermatitis, seborrhoeic dermatitis, actinic dermatitis, pododermatitis, demodicosis, pompholyx dermatitis, lichen simplex chronicus (including Canine acral lick dermatitis and neurodermatitis), digital dermatitis (including bovine digital dermatitis), exfoliative dermatitis (drythroderma), carcinomatous dermatitis, nummular dermatitis, stasis dermatitis, flea allergy dermatitis, otitis, food allergic dermatitis, malassezia dermatitis, intertrigo, perioral dermatitis, dermatomyositis, eczematous dermatitis, photoallergic dermatitis, phototoxic dermatitis, phytophotodermatitis or radiation-induced dermatitis.

22. The method of claim 12, wherein the dermatitis is atopic dermatitis.

23. The method claim 12, wherein the halogenated salicylanilide is selected from rafoxanide, oxyclozanide, closantel and niclosamide or a pharmaceutically acceptable salt, solvate or ester thereof.

24. The method of claim 12, wherein the halogenated salicylanilide is niclosamide or a pharmaceutically acceptable salt or hydrate thereof, optionally niclosamide or a pharmaceutically acceptable salt thereof, for example wherein the halogenated salicylanilide is niclosamide.

25. The method of claim 12, wherein the halogenated salicylanilide is oxyclozanide, or a pharmaceutically acceptable salt or hydrate thereof, optionally oxyclozanide or a pharmaceutically acceptable salt thereof, for example wherein the halogenated salicylanilide is oxyclozanide.

26. The method of claim 12, wherein the halogenated salicylanilide is topically administered to the subject.

27. The method of claim 26, wherein the halogenated salicylanilide is topically administered in the form of a topical composition.

28. The method of claim 27, wherein the topical composition is selected from a topical cream, ointment, gel, paste, foam, solution, suspension, pour-on, spot-on or line-on composition.

29. The method of claim 27, wherein the topical composition comprises the halogenated salicylanilide and a formulation base selected from an oleaginous base (e.g. petrolatum, white petrolatum, yellow ointment or white ointment), an absorption base (e.g. hydrophilic petrolatum or lanolin), a water-removable base (oil in water emulsion); and a water-soluble base (e.g. a polyethylene glycol).

30. The method of claim 27, wherein the topical composition is a non-aqueous composition.

31. The method of claim 27, wherein the topical composition is an aqueous composition.

32. The method of claim 27, wherein the topical composition is a non-aqueous topical composition comprising:
(i) the halogenated salicylanilide (for example selected from niclosamide, rafoxanide, oxyclozanide and closantel), or a pharmaceutically acceptable salt or hydrate thereof; and
(ii) polyethylene glycol (PEG), preferably a PEG with a melting point of less than 40° c.

33. The method of claim 27
wherein the topical composition is a non-aqueous topical composition comprising:
(i) 0.01 to 7.5% (for example, 0.01 to 4.5%, or 0.1 to 3% or about 2%) by weight of the halogenated salicylanilide, or a pharmaceutically acceptable salt or hydrate thereof; and
(ii) at least 70% (for example at least 90%) by weight of a PEG, wherein the average molecular weight of the PEG is 600 or less (for example less than 600 or from about 200 to about 600 or about 400).

34. The method of claim 27, wherein the topical composition further comprises a non-polymeric glycol (e.g. propylene glycol).

35. The method of claim 27, wherein the halogenated salicylanilide is dissolved or partially dissolved in the composition.

36. The method of claim 27, wherein the halogenated salicylanilide is present in the topical composition at a concentration of from 0.5 mg/ml to 200 mg/ml, optionally at a concentration of from 50 mg/ml to 100 mg/ml, for example 100 mg/ml.

37. The method of claim 12 wherein the halogenated salicylanilide is topically administered 1, 2, 3 or 4 times per day over a period of 1 week or more, optionally over a period of 2 weeks or more, 3 weeks or more, 4 weeks or more, 6 weeks or more, 12 weeks or more, 6 months or more, or 1 year or more.

38. The method of claim 12, wherein the subject is a companion animal, preferably a dog or cat, more preferably a dog.
